(19) United States
(12) Patent Application Publication
(10) Pub. No.: US 2021/0260527 A1
(43) Pub. Date: Aug. 26, 2021

(54) DEVICES, FACILITIES, METHODS AND COMPOSITIONS FOR CARBON DIOXIDE CAPTURE, SEQUESTRATION AND UTILIZATION

(71) Applicant: THRI LLC, Austin, TX (US)

(72) Inventor: Claude Steven McDaniel, Austin, TX (US)

(73) Assignee: THRI LLC, Austin, TX (US)

(21) Appl. No.: 17/237,798

(22) Filed: Apr. 22, 2021

Related U.S. Application Data
(62) Division of application No. 16/673,360, filed on Nov. 4, 2019.
(60) Provisional application No. 62/840,167, filed on Apr. 29, 2019.

(51) Int. Cl. B01D 53/85; B65D 23/08; C12M 1/00; C12M 1/107

(52) U.S. Cl. CPC B01D 53/85; B65D 23/0814; B01D 2251/95; C12M 23/36; C12M 21/02

(57) ABSTRACT

Disclosed herein are carbon dioxide capture devices, facilities, methods, and compositions. Specifically disclosed herein are devices, facilities, compositions and methods to capture carbon dioxide from the Earth's atmospheric air for providing long-term sequestration of the captured carbon and/or utilization thereof. To this end, a carbon dioxide capture device configured in accordance with one or more embodiments of the present invention can comprise a coating substrate having at least one coatable surface and a carbon dioxide capture coating composition on a coatable surface of the coating substrate. The carbon dioxide capture coating composition preferably comprises a coating material and a photosynthetic organism, wherein the photosynthetic organism is at least one of admixed within the coating material and on an exposed surface of the coating material. The coating material can deliver all or a portion of water to the photosynthetic organism necessary for sustaining photosynthetic activity of the photosynthetic organism.

### CROSS-REFERENCE TO RELATED APPLICATIONS

This continuation patent application claims priority from co-pending United States Non-provisional patent application having Ser. No. 16/673,360, filed 4-Nov. 2019, entitled “APPARATUSES, METHODS AND COMPOSITIONS FOR CARBON DIOXIDE CAPTURE”, which claims priority from co-pending United States Provisional Patent Application having Ser. No. 62/840,167, filed 29-Apr. 2019, entitled “APPARATUSES, METHODS AND COMPOSITIONS FOR CARBON DIOXIDE CAPTURE”, all having a common applicant herewith and being incorporated herein in their entirety by reference.

### FIELD OF THE DISCLOSURE

The disclosures herein relate generally to carbon dioxide capture, sequestration and utilization and, more particularly, the disclosures herein are directed to devices, facilities, methods and compositions for capturing carbon dioxide (CO₂) from a gaseous environment such as, for example, the earth's atmosphere to enable long-term storage, utilization or atmospheric balancing of such captured carbon dioxide (i.e., carbon dioxide sequestration, captured carbon utilization and/or the like).

### BACKGROUND

It is well-known that carbon dioxide is a by-product of both naturally-occurring activities and man-made activities. Examples of such naturally-occurring activities include, but are not limited to, animal respiration, decomposition of formerly living organisms, weathering of carbonate rocks, volcanic eruptions, plant (e.g., forest) fires and the like. Examples of such man-made activities include, but are not limited to, fossil fuel use, intentional burning of biomass (e.g., wood stoves, intentional or unintentional forest fires), cement production, ammonia production and the like. However, as shown in FIG. 1, the current increase in carbon dioxide 12 in Earth's atmosphere (“air”) 14 surrounding the Earth 16 (i.e., atmospheric carbon dioxide 12) is being driven by release of additional carbon dioxide 18 by mostly terrestrial, man-made activities, particularly in industrialized regions such, for example, as North America 20, South American 22, and Western Europe 24.

Photosynthesis from land-based plants and from ocean-bound algae (i.e., a photosynthetic microorganism) are the two primary naturally-occurring processes for removing carbon dioxide from Earth's atmosphere (i.e., ambient air surrounding the earth). This naturally-occurring photosynthetic conversion of carbon dioxide requires water (e.g., ambient environmental moisture) and sunlight to convert available carbon dioxide in the air to oxygen (O₂) and carbohydrates (e.g., saccharides). Of these two naturally occurring sources of photosynthesis, as illustrated in FIG. 2, ocean-bound algae 30 and terrestrial plants 32 (e.g., trees within forests) provide the majority of the photosynthetic capture (“uptake”) of atmospheric carbon dioxide 12 from the air 14 surrounding the Earth 16 that results in release of oxygen 34 into the air 14. More specifically, as shown in FIG. 3, the ocean-bound algae 30 is in the form of a thin layer floating at/near the surface of ocean water 42. The vast size of the earth's oceans 42 provides a substantial surface area by which these ocean-bound algae 30 can perform the majority (e.g., about 70%) of photosynthetic capture of atmospheric carbon dioxide 12 and release of oxygen 34 into surrounding air 14.

However, as a result of the sheer magnitude of carbon-dioxide resulting from man-made activities, naturally-occurring photosynthetic conversion of carbon dioxide has for many decades been unable to mitigate the amount of carbon-dioxide produced by man-made activities that is being delivered into the earth's atmosphere. As a result, excess amounts of carbon dioxide have built up in the earth's ambient air. For example, it is estimated that the amount of carbon dioxide in the Earth's ambient air has increased from 280 parts per million (“ppm”) in the 1700s to 411 ppm in March 2019 [Eggleton, T. (2012) “A Short Introduction to Climate Change,” Cambridge University Press, p. 53; “Up-to-date weekly average CO₂ at Mauna Loa” Earth System Research Laboratory Global Monitoring Division, Mauna Loa, Hi., Retrieved Mar. 28, 2019]. Additionally, to make matters worse, approximately 50 gigatons of additional carbon dioxide equivalents (e.g., greenhouse gasses such as carbon dioxide, methane, nitrous oxide, hydrofluorocarbons, perfluorocarbons, sulfur hexafluoride) are released into the Earth's ambient air per year primarily from fossil fuel usage (Bridging the Emissions Gap: A UNEP Synthesis Report, Nairobi, Kenya: United Nations Environment Programme (UNEP), November 2011).

It is well known that carbon dioxide represents about 80% of “Greenhouse Gas”, with methane, nitrous oxide and floriated gases representing the balance. It is also well-known that these Greenhouse Gas are the contributing factor to global warming and the associated climate-change around the globe (i.e., severe storms, warming of the oceans, melting of glaciers, rising sea levels and the like). The current level of Greenhouse Gas and their projected rate of increase have led experts in the field of climatology to the conclusion that, if left unchecked, further increases in Greenhouse Gases (i.e., which consists largely of carbon dioxide) will result in irreversible climatic changes and resulting catastrophic effects of climate change on people, property and nature.

To combat the current level of Greenhouse Gas and their projected rate of increase, various forms of negative emission technologies have begun to be developed. A negative emission technology removes (i.e., captures) carbon dioxide equivalents from the atmosphere, where they may be sequestered (“stored”) for long periods of time (i.e., years, decades, centuries or longer) (Minx, J. C. et al., Environmental Research Letters 13(6):063001). Negative emission technologies can include enhanced carbon sinks that provide for long-term storage of the removed carbon dioxide (and/or other component(s) of Greenhouse Gas). Examples of such enhanced carbon sinks include, but are not limited to, storage of captured carbon dioxide underneath the surface of the earth, conversion of captured carbon to useful solid or liquid material such as plastics, carbon fibers, biofuel, or carbon-based chemicals and the like.

To combat the current level of Greenhouse Gas and their projected rate of increase, various forms of negative emission technologies have begun to be developed. A negative emission technology removes (i.e., captures) carbon dioxide equivalents from the atmosphere. Examples of negative emission technologies include geological carbon sequestration, direct carbon dioxide capture (also sometimes referred to as “carbon dioxide capture”) from the air, bioenergy carbon capture and storage, coastal blue carbon capture, terrestrial carbon sequestration, and carbon mineralization of carbon dioxide. Geological carbon storage refers to pumping captured carbon dioxide from other negative emission technologies into the pores of subsurface rock formations. Direct carbon capture from air concentrates atmospheric carbon dioxide for long-term storage or use as a raw material in a product. Bioenergy carbon capture entails capturing carbon dioxide and using the captured carbon dioxide in a biomass that is used in fuels. Terrestrial carbon sequestration generally entails increasing forestation and agricultural soil carbon content, while coastal blue carbon sequestration focuses on similar processes at tidal or wetland areas. Carbon mineralization refers to atmospheric or captured carbon dioxide being contacted with basaltic or ultramafic rocks to undergo a chemical reaction convert the carbon dioxide into a chemical solid. Carbon capture and utilization (CCU) is the process of capturing carbon dioxide (CO₂) to be recycled for further usage. Carbon capture and utilization may offer a response to the global challenge of significantly reducing greenhouse gas emissions from major stationary (industrial) emitters.

In much the same way that the magnitude of photosynthetic conversion of atmospheric carbon dioxide by ocean-bound algae is limited by the surface area of the ocean having algae exposed to the atmosphere (i.e., the atmosphere expose surface), known negative emissions technologies are similarly limited in their efficiency and effectiveness for mitigating carbon dioxide (and other Greenhouse Gases) by their available footprint (i.e., terrestrial or otherwise surface area coverage). Surface area occupied by facilities housing these negative emissions technologies must do so in a manner that is practically feasible from societal and financial perspectives. Therefore, a negative emissions technological solution that simulates the unequalled efficiency and effectiveness of naturally-occurring sources of photosynthesis (e.g., ocean-bound algae and/or terrestrial plants), but in a manner that provides for markedly greater surface area-to-volume practicalities, would be advantageous, desirable and useful.

### SUMMARY OF THE DISCLOSURE

Embodiments of the present invention are directed to a negative emissions technology that simulates the efficiency and effectiveness of naturally-occurring sources of photosynthesis (e.g., ocean-bound algae and/or terrestrial plants) in a manner that provides for markedly greater surface area-to-volume practicalities. More specifically, embodiments of the present invention comprise photosynthetic organism-containing polymeric materials (e.g., coatings) adapted for being formed on surfaces of vertically extending substrates (i.e., algae-coated vertical surfaces). In one or more embodiments, algae are a preferred photosynthetic organism. In one or more embodiments, the vertically extending substrates can be located within an interior space of a carbon dioxide capture facility in accordance with embodiments of the present invention, which is also referred to herein as a vertical algae farm or forest. The carbon dioxide capture facility can be adapted for providing environmental conditions within the interior space thereof that are conducive to the life, growth and carbon capture of the photosynthetic organism. Advantageously, on a unit-volume basis, a carbon dioxide capture facility in accordance with one of more embodiments of the present invention provides a total area of photosynthetic organism-coated vertical surfaces magnitudes of order greater than the atmosphere-exposed surface of a corresponding volume of ocean or forest. In this manner, embodiments of the present invention enable a marked increase in the amount of photosynthetic conversion of carbon dioxide for a given volume of space as compared to ocean-bound algae or terrestrial plants and, thus, advantageously overcome shortcomings associated with other negative emission technologies.

In preferred embodiments, a coating comprising a photosynthetic organism maintains hydration of the photosynthetic organism by retaining water (e.g., moisture) in the coating, and/or absorbing water vapor from the atmosphere or a gas in contact with the coating. The water retained and/or absorbed comprise potable water and/or greywater. In other embodiments, water (e.g., moisture) is not substantively (e.g., little or no water) gained, uptaken, and/or wicked from a substrate, undercoat, or combination thereof that contacts the coating comprising a photosynthetic organism. For example, the coating comprising a photosynthetic organism may be one or more layers upon a non-porous and/or non-wicking substrate such as plastic, glass, or the like. Further, in certain embodiments materials in the coating (e.g., a carbon containing molecule created by photosynthetic capture of carbon dioxide by a photosynthetic organism) is not partly or fully extracted from the coating by wicking by aqueous substances contacting the substrate, coating, undercoat, or a combination thereof. In other embodiments, the exposure of the coating may be accomplished using a water-enriched environment such as by misting, vaporizing, spraying, atomizing, drizzling or condensation. Using such a water-enriched environment, it is possible to additionally deliver nutrients, micronutrients, trace minerals, and coating-modifying substances to the exposed surfaces of the carbon capture coating.

In one embodiment of the present invention, a carbon dioxide capture facility is provided in the form of an algae farm (or forest). The algae farm provides photosynthetic capture of carbon dioxide from air surrounding the Earth (i.e., atmospheric carbon dioxide) with a resulting release of oxygen into the atmosphere. Each algae farm (or forest) is an example of a carbon dioxide capture facility configured in accordance with one or more embodiments of the present invention. Each algae farm comprises algae-containing coating on surface area of a coating substrate. Through exposure to air, light and water, the algae-containing coating provides naturally-occurring photosynthetic conversion of atmospheric carbon dioxide (CO₂) within the air. Such photosynthetic conversion results in the production of oxygen and carbon-containing by-product (i.e., carbohydrate such as saccharide). The oxygen is delivered from the algae farm back into the air and the carbon-containing by-product can be utilized and/or sequestered (i.e., stored) such as, for example, by delivery into the earth (e.g., deep well, storage cavern, etc.), by delivery into subsea environment, and/or utilization in the production of products (e.g., building products, fuels, food stocks, and the like). Accordingly, the algae farm can advantageously provide a volumetric efficiency (and associated terrestrial footprint) and that provides a marked increase in the amount of photosynthetic conversion of carbon dioxide for a given volume of space as compared to naturally-occurring sources of photosynthesis (e.g., ocean-bound algae and/or terrestrial plants).

In another embodiment of the present invention, a carbon dioxide capture device comprises one or more coating substrates that are each in the form of a generally planar shaped article (e.g., a sheet of material), wherein at least one side of each coating substrate is coated with a photosynthetic organism containing coating. Carbon-containing by-product is generated by the photosynthetic organism containing coating. The carbon dioxide capture devices are configured for being arranged within an interior space of an encasement of a carbon dioxide capture facility and/or carbon dioxide capture apparatus in a manner that provides sufficient space for the photosynthetic organism containing coating to be exposed to necessary amounts of atmospheric carbon dioxide, light and water (e.g., atmospheric moisture). The carbon dioxide capture devices are arranged for optimizing surface area of the coating substrate within the interior space of the encasement of the carbon dioxide capture facility and/or carbon dioxide capture apparatus, while still providing for such necessary atmospheric carbon dioxide, light and water to be present at the photosynthetic organism containing coating of the carbon dioxide capture devices. Advantageously, such a carbon dioxide capture device provides for high levels of coated surface area, which is achieved through the photosynthetic organism containing coating of being relatively lightweight and thin (e.g., relative to a layer of ocean bound algae). In turn, this relatively lightweight and thin coating permits the coating substrate to also be relatively lightweight and thin.

In another embodiment of the present invention, a method in accordance with an embodiment of the present invention is adapted for providing capture of carbon dioxide from the air (e.g., atmospheric carbon dioxide). The method is preferably adapted for operating an algae farm (or forest) for capturing atmospheric carbon dioxide. To this end, the method can employ carbon dioxide capture devices and apparatuses described in accordance with the disclosures herein. The method includes a plurality of operations that jointly provide for the aforementioned capture of carbon dioxide from air. The method provides functionalities including, but not limited to, providing carbon dioxide capture facility (i.e., system) components that enable photosynthetic conversion of carbon dioxide, providing operating conditions that enable photosynthetic conversion of carbon dioxide, harvesting carbon-containing by-product resulting from the photosynthetic conversion of carbon dioxide (e.g., from an algae farm), and implementing utilization and/or sequestration of the carbon-containing by-product.

In one or more embodiments, a carbon dioxide capture apparatus is characterized by a plurality of discrete carbon dioxide capture devices that are retained within a device retaining structure such as by being stacked in an end-to-end fashion and/or side-by-side fashion within an interior space of the device retaining structure. Each of the carbon dioxide capture devices includes a substrate having one or more surface areas with a photosynthetic organism (e.g., algae) containing coating formed thereon (i.e., the “coating substrate”). In one or more embodiments, the coating substrates are plastic containers such as single-use beverage containers recycled from a waste stream. The device retaining structure can be a sleeve or tube made from a material such as, for example, glass or plastic. For enabling light transmission, the respective materials from which the coating substrate and the device retaining structure are made are light transmissive (e.g., fully or partially transparent). For enabling water vapor and air transmission, the coating substrate and the device retaining structure each have one or more openings therein for enabling airflow therethrough.

In one or more embodiments, a carbon dioxide capture device comprises a coating substrate and a carbon dioxide capture coating composition. The coating substrate has at least one coatable surface. The coating substrate inhibits fluid transmission therein. The carbon dioxide capture coating composition is provided on a coatable surface of the coating substrate. The carbon dioxide capture coating composition comprises a coating material and a photosynthetic organism. The photosynthetic organism is at least one of admixed within the coating material and on an exposed surface of the coating material. The coating material delivers water to the photosynthetic organism.

In one or more embodiments, a carbon dioxide capture facility comprising a plurality of carbon dioxide capture devices and a retaining structure having the carbon dioxide capture devices engaged therewith. Each of the carbon dioxide capture devices includes a coatable surface. The coating substrate inhibits fluid transmission therein. The coatable surface of each of the carbon dioxide capture devices includes a carbon dioxide capture coating composition thereon. The carbon dioxide capture coating composition comprises a coating material and a photosynthetic organism. The coating material comprises at least one of a hydrogel polymer and a hydrophilic polymer for enabling the coating material to deliver water to the photosynthetic organism. The photosynthetic organism is at least one of admixed within the coating material and on an exposed surface of the coating material. The retaining structure retains each of the carbon dioxide capture devices in one of fixed and movable relation to each adjacent one of the carbon dioxide capture devices.

Implementing carbon dioxide capture and capture of other types of Greenhous Gases is provided for by embodiments of the present invention through use of a coating composition comprising a coating material and a photosynthetic organism, where the coating composition is provided on a coating substrate. In preferred embodiments, the coating composition is a carbon dioxide capture coating composition. Coating compositions configured in accordance with one or more embodiments of the present invention can have a plurality of properties and attributes that advantageously enable the coating composition to support life of the photosynthetic organism, promotes carbon dioxide capture by the photosynthetic organism and enabling implementation of such coatings in an environmentally-friendly manner. As set forth in greater detail herein, examples of these properties and attributes include being highly translucent, being non-toxic to humans, being environmentally benign, being non-toxic to Cyanobacteria and other photosynthetic organisms, supporting growth and maintenance of Cyanobacteria and other photosynthetic organisms, allowing high rates of atmospheric gas exchange, being highly and repeatedly hydrated after curing and easily re-hydrated over time, providing self-hydration when on a moisture impermeable coating substrate, adhering to smooth surfaces, preventing excess photon flux from photobleaching of Cyanobacteria and other photosynthetic organisms, being flexible and resilient enough to allow for swelling by accumulated by-products of photosynthesis, using renewable resources for formulation components, being readily depolymerized and repolymerized (i.e., being re-useable), being highly refractive to allow maximum light capture and dissemination, and combinations thereof.

In one or more embodiments, the carbon dioxide capture coating composition delivering water to the photosynthetic organism may include transmitting water from a water source in contact with the carbon dioxide capture coating composition, retaining the water in the carbon dioxide capture coating composition, absorbing water vapor from a gas in contact with the carbon dioxide capture coating composition, or a combination thereof.

In one or more embodiments, the coating substrate may comprise a non-porous substrate, a non-wicking substrate, a water impermeable substrate, or a combination thereof.

In one or more embodiments, the coating substrate may inhibit the transmission of water through a thickness thereof and along a length and width thereof.

In one or more embodiments, the coating material may comprise at least one of a hydrogel polymer and a hydrophilic polymer.

In one or more embodiments, the hydrogel polymer may comprise at least one of alginate, polyacrylamide, and polyacrylate.

In one or more embodiments, the hydrophilic polymer may comprise at least one of hydroxy ethyl cellulose, carboxy methyl cellulose, xanthan gum, guar gum, agar, agarose, gelatin, and polyvinyl acetate.

In one or more embodiments, the coating material may comprise a combination of alginate and xanthan gum.

In one or more embodiments, the carbon dioxide capture coating composition may provide an hourly carbon dioxide capture rate of at least about 0.25 millimoles per square meter of the coating substrate.

In one or more embodiments, the carbon dioxide capture coating composition may provide an hourly carbon dioxide capture rate of between about 0.1 and 8.0 millimoles per square meter of the coating substrate.

In one or more embodiments, the carbon dioxide capture coating composition may provide an hourly carbon dioxide capture rate of between about 0.5 and 6.0 millimoles per square meter of the coating substrate.

In one or more embodiments, the carbon dioxide capture coating composition may provide an hourly carbon dioxide capture rate of between about 1.0 and 3.0 millimoles per square meter of the coating substrate.

In one or more embodiments, the photosynthetic organism is at least one cyanobacteria of the group comprising Synechococcus leopoliensis, Spirulina sp., Synechocystis sp., Gloeocapsa alpicola, Agmenellum quadruplicatum, Anabaena sp., Anabaena variabilis, and Nostoc muscorum.

In one or more embodiments, the carbon dioxide capture coating composition may be about 1 μm to 2000 μm in thickness and the coating substrate may be about between about 5 μm to about 250 mm in thickness.

In one or more embodiments, the coating composition may be applied to the substrate in a single layer, or in multiple layers so long as the layered coatings support the requisite levels of growth and carbon capture.

In one or more embodiments, the coating substrate may be in the form of a tubular body made of plastic and the carbon dioxide capture coating composition is on a surface within an interior space of the tubular body.

In one or more embodiments, the coating substrate may be in the form of a beverage bottle having a beverage-receiving space and the carbon dioxide capture coating composition may be on a surface of the beverage bottle within the beverage-receiving space.

In one or more embodiments, a quantity and arrangement of the carbon dioxide capture devices may be engaged with the retaining structure provides an annualized CO₂ capture rate of at least about 0.01 metric tons per square meter of plan-view area of the retaining structure.

In one or more embodiments, a quantity and arrangement of the carbon dioxide capture devices may be engaged with the retaining structure provides an annualized CO₂ capture rate of about 0.5 to about 3.0 metric tons per square meter of plan-view area of the retaining structure.

In one or more embodiments, a quantity and arrangement of the carbon dioxide capture devices may be engaged with the retaining structure provides an annualized CO₂ capture rate of about 0.75 to about 2.0 metric tons per square meter of plan-view area of the retaining structure.

In one or more embodiments, a quantity and arrangement of the carbon dioxide capture devices may be engaged with the retaining structure provides an annualized CO₂ capture rate of at least about 1.0 metric tons per square meter of plan-view area of the retaining structure.

In one or more embodiments, the carbon dioxide capture devices being engaged with the retaining structure may include the carbon dioxide capture devices being arranged in a side-by-side arrangement and the retaining structure retaining at least a portion of the carbon dioxide capture devices in movable relation to one or more other ones of the carbon dioxide capture devices includes a first one of the carbon dioxide capture devices being laterally translatable with respect to a second one of the carbon dioxide capture devices.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a diagrammatic view showing carbon dioxide in Earth's atmosphere, with release of additional carbon dioxide due to man-made activities.

FIG. 2 is a perspective view showing ocean-based alga and land-based plant performing photosynthetic conversion of atmospheric carbon dioxide into oxygen that is released into Earth's atmosphere.

FIG. 3 is a detailed diagrammatic view showing ocean-based alga performing photosynthetic conversion of atmospheric carbon dioxide into oxygen that is released into Earth's atmosphere.

FIG. 4 is a diagrammatic view showing carbon dioxide capture facilities (e.g., algae farms and forests) in accordance with an embodiment of the present invention, which are performing photosynthetic conversion of atmospheric carbon dioxide into oxygen that is released into Earth's atmosphere.

FIG. 5 is a diagrammatic view showing a carbon dioxide capture facility in accordance with an embodiment of the present invention.

FIG. 6 is a fragmentary diagrammatic view showing carbon dioxide capture devices of the carbon dioxide capture facility shown in FIG. 5.

FIG. 7 is a flow diagram showing a method in accordance with an embodiment of the present invention, which is adapted for providing capture of carbon dioxide from air.

FIG. 8 is a diagrammatic view showing a technique for a photosynthetic organism containing coating's delamination from a coating substrate configured in accordance with an embodiment of the present invention.

FIG. 9 is a diagrammatic view showing a carbon dioxide capture apparatus in accordance with an embodiment of the present invention.

FIG. 10 is a diagrammatic view showing a process for implementing harvesting of carbon-containing by-product from a carbon dioxide capture device of a carbon dioxide capture apparatus as shown in FIG. 9.

FIG. 11 is a diagrammatic view showing a carbon dioxide capture facility comprising a plurality of carbon dioxide capture apparatuses of the configuration shown in FIG. 9.

FIG. 12 is a diagrammatic view showing attributes of associated with implementation of carbon dioxide capture and sequestration in accordance with carbon dioxide capture facilities, apparatuses, and devices configured in accordance with embodiments of the present invention.

FIG. 13 is a graph of CO₂ capture by a carbon dioxide capture device comprising a xanthan gum/Synechococcus leopoliensis coating as measured over 1 hour on days 0, 5 and 9 after creation of the carbon dioxide capture device in accordance with embodiments of the present invention.

FIG. 14 is a graph of CO₂ capture by a carbon dioxide capture device comprising an alginate/Synechococcus leopoliensis coating as measured over 1 hour on days 0, 5 and 9 after creation of the carbon dioxide capture device in accordance with embodiments of the present invention.

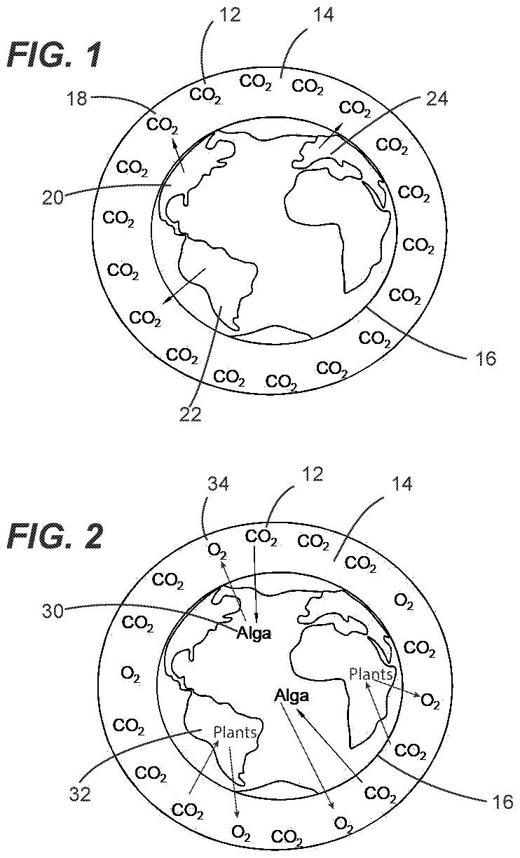

### DETAILED DESCRIPTION

Embodiments of the present invention are directed to apparatuses, methods and material composition that reduce the amount of atmospheric carbon dioxide by increasing the amount of photosynthetic carbon dioxide capture and sequestration utilizing a photosynthetic organism (e.g., an algae, a photosynthetic bacteria) in and/or upon a material capable of sustaining the life and photosynthetic activity of the organism (“the life supporting material”). Preferred embodiments of such a material combined with the photosynthetic organism (i.e., a coating composition) typically comprise a polymeric material, a coating, a hydrogel, an adhesive, or a combination thereof, all referred herein to be a coating when applied on a substrate as a layer. As used herein, the organism(s) selected for combination with the material(s) will be used as the prefix for the combination, such as for example, algae combined with a material applied to a surface as a thin layer would be designated an “algae-containing coating” or “algae coating,” and an algae-containing coating(s) and device(s) will be used as an exemplary but non-limiting embodiment described herein.

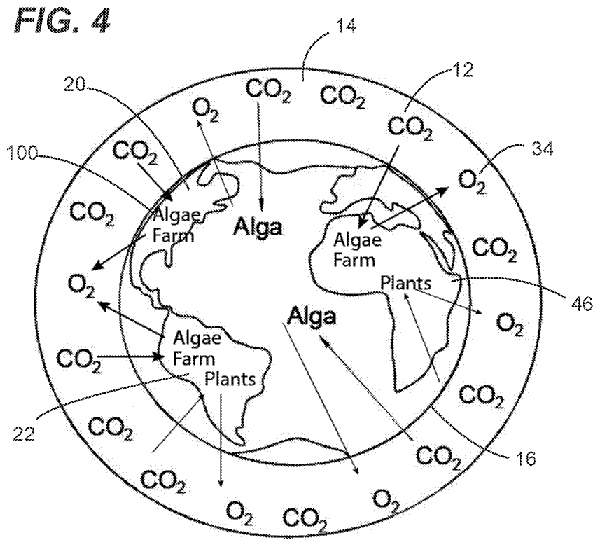

Referring to FIG. 4, a carbon dioxide capture facility configured in accordance with one or more embodiments of the present invention may be in the form of one or more algae farms 100 (or forests). Alternatively or additionally, a carbon dioxide capture facility configured in accordance with one or more embodiments of the present invention may be in the form of one or more algae forest. An algae farms can have an intended objective of harvesting of an oxygen or carbon-containing or nitrogen-containing by-product produced by carbon dioxide or nitrogen capture devices thereof, whereas an algae forest can have the objective of sequestering a carbon-containing by-product produced by carbon dioxide capture devices thereof. In some embodiments, algae farms harvest a carbon-containing by-product for carbon capture utilization (i.e., creation of a product). As shown, each algae farm 100 provides photosynthetic capture of atmospheric carbon dioxide 12 from the air 14 surrounding the Earth 16 with a resulting release of oxygen 34 into the atmosphere. Each algae farm 100 (or algae forest) is an example of a carbon dioxide capture facility configured in accordance with one or more embodiments of the present invention, which may be located in various land-based regions such as, for example, North America 20, South America 22, and Africa 46. It is also disclosed herein that one or more carbon dioxide capture facilities configured in accordance with embodiments of the present invention may be located on a body of water (e.g., an ocean vessel) or in the air (e.g., on an aircraft). As discussed herein in greater detail below, preferred embodiments of a carbon dioxide capture facility may comprise a plurality of carbon dioxide capture devices and/or materials organized in a specific manner and/or a specific location for promoting photosynthetic carbon dioxide capture and subsequent long-term storage thereof.

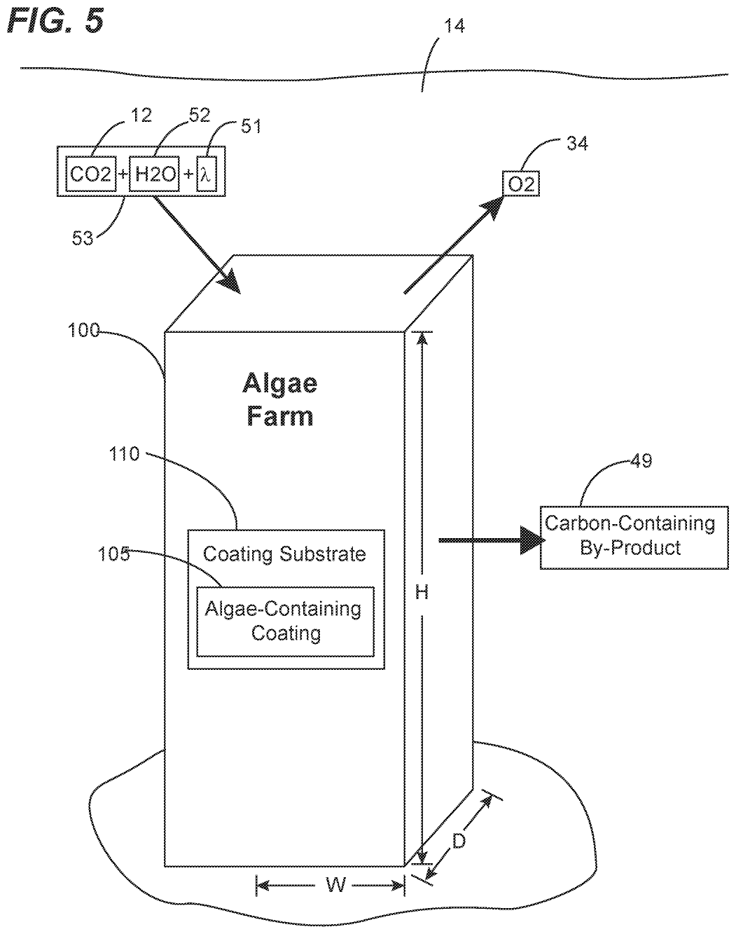

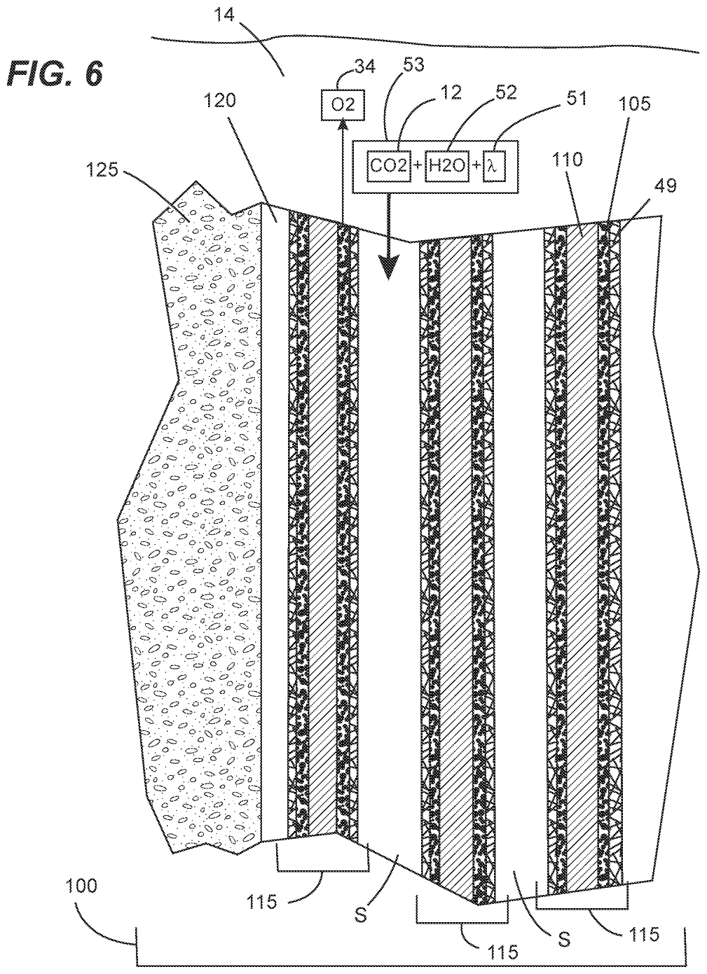

As illustrated in FIGS. 5 and 6, each algae farm 100 comprises algae-containing coating 105 (i.e., a carbon dioxide capture coating composition) on surface area of coating substrate 110. The algae in the algae-containing coating 105 is a photosynthetic organism. Through exposure to carbon dioxide (CO₂) 12, light (λ) 51 and water (H₂O) (e.g., water vapor) 52 (i.e., exposure conditions 53), the algae-containing coating 105 provides naturally-occurring photosynthetic conversion of atmospheric carbon dioxide 12 (CO₂) within the air 14. Such photosynthetic conversion results in the production of oxygen 34 and carbon-containing by-product 49 (i.e., carbohydrate such as saccharide). The oxygen 34 is delivered from the algae farm 100 back into the air 14 and the carbon-containing by-product 49 can be sequestered (i.e., stored) such as, for example, by delivery into the earth (e.g., deep well, storage cavern, etc.), by delivery into subsea environment, and/or carbon capture utilization in the production of products (e.g., building products, fuels, food stocks, and the like).

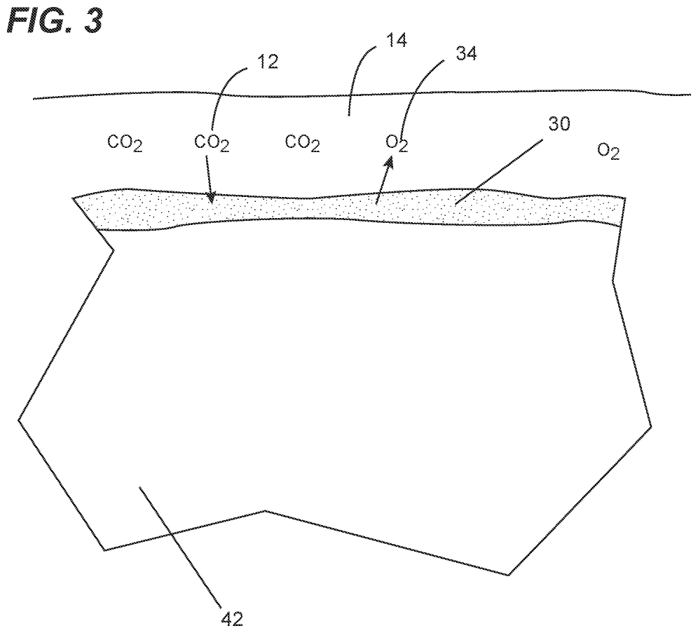

Advantageously, the algae farm 100 can provide a surface area and/or volumetric efficiency that provides a marked increase in the amount of photosynthetic conversion of carbon dioxide for a given volume of space and terrestrial area as compared to ocean-bound algae. More specifically, the algae farm 100 has an interior space having volumetric characteristics defined at least partially by a depth D, a width W and a height H (i.e., jointly, the algae farming space). Within the algae farming space, the coating substrate 110 is arranged to offer magnitudes of order greater surface area of the coating substrate 110 for a given volume of space as compared to an equivalent volume of ocean water containing a layer of ocean-bound algae (e.g., as shown in FIG. 3). In this manner, the algae farm 100 correspondingly and advantageously offers magnitudes of order greater photosynthetic conversion surface area than does an equal volume of ocean water.

A carbon dioxide capture facility in accordance with one or more embodiments of the present invention simulates naturally-occurring sources of photosynthesis (e.g., ocean-bound algae and/or terrestrial plants). For example, like a mature forest, an inventive carbon dioxide capture facility can provide photosynthesis over a relatively long duration of time (e.g., years or decades), which is a highly desirable attribute of implementing capture of carbon dioxide. Advantageously, an inventive carbon dioxide capture facility exhibits carbon-dioxide capture rates not attainable by a mature forest or other naturally-occurring sources of photosynthesis. To this end, an inventive carbon dioxide capture facility densities surface area of a photosynthetic organism coated substrate (i.e., carbon dioxide capture devices) that provides such photosynthesis relative to a plan-view area (e.g., terrestrial area) occupied by the carbon dioxide capture facility.

Mature forests are reported to exhibit a carbon capture rate of about 2.2-9.5 metric tons of CO₂ per acre per year relative to development by known afforestation practices and of about 1.1-7.7 metric tons of CO₂ per acre per year relative to development by known reforestation practices. (U.S. Environmental Protection Agency, Office of Atmospheric Programs, Greenhouse Gas Mitigation Potential in U.S. forestry and Agriculture, EPA 430-R-05-006, Washington, D.C., November 2005, Table 2-1.) As also discussed below in the Examples (e.g., Example 3), carbon dioxide capture devices in accordance with one or more embodiments of the present invention have been shown based on current levels of development to provide carbon capture rates of at least about 0.25 mmol of CO₂ per square meter per hour. As discussed below in the Examples (e.g., Example 16), carbon dioxide capture devices in accordance with one or more embodiments of the present invention have been shown based on current levels of development to provide surface area densification of at least about 30 times that of a plan view (i.e., footprint) area occupied by the carbon dioxide capture devices (e.g., about 30 square meters of coated surface per square meter of plan view (e.g., terrestrial footprint area covered by carbon capture devices) area occupied by such carbon dioxide capture devices). In one or more other embodiments, carbon dioxide capture devices in accordance with one or more embodiments of the present invention can have been shown based on current levels of development to provide surface area densification of at least about 200 times that of a plan view area occupied by the carbon dioxide capture devices (e.g., for nested sleeves, about 200 square meters of coated surface per square meter of plan view (e.g., terrestrial) area occupied by such carbon dioxide capture devices). Accordingly, in some embodiments (see Example 17), such surface area densification results in a unit area carbon dioxide capture rate at least about 5 times greater and as much as 25 times greater than that of a comparable terrestrial area of mature forest.

Still referring to FIGS. 5 and 6, upon irradiation by wavelengths of light (λ) 51 that promote photosynthesis by the alga cells of the algae-containing coating 105, the algae cells uptake of carbon dioxide 12 and water 52 in order to photosynthetically capture carbon dioxide by converting carbon dioxide gas into a non-gas (e.g., a solid or a liquid) carbon-containing by-product 49. Oxygen 34 is released as a result of the photosynthetic chemical reaction of the algae farm 100. The carbon-containing by-product 49 produced by photosynthesis is generally a saccharide (“sugar”) that may be metabolized into other forms of the carbon-containing by-product 49 [e.g., various sugar(s) (e.g., glucose, sucrose), polysaccharide(s) (e.g., cellulose), and/or carbon containing by-product such as a protein, a lipid, etc.].

In preferred embodiments, the carbon-containing by-product 49 is sequestered in an environment wherein degradation of the carbon-containing by-product is minimalized and/or the carbon-containing by-product (and possibly the coating(s) used in the algae farm) is converted into another useful product (i.e., captured carbon utilization). For example, the carbon-containing by-product 49 may be released from within a photosynthetic cell of the algae-containing coating 105 into a surrounding exterior environment (e.g., within the algae farm 100 or structure/environment exterior to the algae farm 100). In one specific example, the carbon-containing by-product 49 is retained within the molecular and/or cellular structure of the algae-containing coating. In one specific example, the carbon-containing by-product 49 is formed a separate layer adjacent to and/or within the algae-containing coating 105. The carbon-containing by-product 49 may then be more readily harvested by fluid-extraction (e.g., via a solvent such as, for example, water) and/or physical removal (e.g., delamination of a separate layer comprising the carbon-containing by-product 49). In one or more embodiments, the algae-containing coating 105 preferably remains on the coating substrate 110 after harvesting of the carbon-containing by-product 49 thereby enabling continued photosynthetic production of additional carbon containing by-product 49. Examples of the carbon-containing by-product can include O₂, glucose or possibly mannose, generation of additional algae, biomass for soil modifiation, cellulose (e.g., bacterial (microcrystallin) cellulose), sulfonated polysaccharide cosmetic moisturizer, bio-modified polysaccahride, fuel feedstock as sugars, fuel feedstocks as ammonia, fertilizer, carbon credits via several mecahisms.

In one or more other embodiments, both the algae-containing coating 105 and the carbon containing by-product 49 may be jointly harvested and used (e.g., sequestered). In one specific embodiment where the algae cells have reduced productivity due to cell death, coating degradation/chemical leaching and the like, harvesting of a current layer of algae-containing coating 105 enables another layer (i.e., a new layer) of algae-containing coating to thereafter be formed on the coating substrate 110. An algae forest may generally and desirably have a much longer duration of use of carbon capture devices thereof before harvesting or termination of use of a current instance of coating thereon than will an algae farm.

As best shown in FIG. 6, an arrangement of carbon dioxide capture devices 115 of one or more of the algae farms 100 (or algae forests) discussed above in reference to FIGS. 4 and 5 is shown. In accordance with a preferred embodiment, the carbon dioxide capture devices 115 are disposed within an interior space 120 of an encasement 125 of the algae farm 100. Each of the carbon dioxide capture devices 115 comprises one or more coating substrates 110 that are each in the form of a generally planar shaped article (e.g., a sheet of material). A least one side of each coating substrate 110 is coated with an algae-containing coating 105 (i.e., a coating comprising a photosynthetic organism). Carbon-containing by-product 49 is shown to form a layer of material on the surface of the algae-containing coating 105, which may occur in the case of a cell that extrudes a carbon-containing by-product into the exterior environment. The carbon-containing by-product 49 may be retained within the body of the algae-containing coating 105 as well and/or form a surface layer upon the algae-containing coating 105.

The carbon dioxide capture devices 115 are arranged within the encasement 125 to provide sufficient separation S for the algae-containing coating 105 to be exposed to necessary amounts of atmospheric carbon dioxide (CO₂) 12, light (λ) 51 and water (H₂O) (e.g., water vapor) 52. In one or more embodiments, such an arrangement may entail separating the carbon dioxide capture devices 115 apart from each other with space sufficient for necessary atmospheric carbon dioxide (CO₂)12, light (λ) 51 and water (H₂O) 52 to be present at the algae-containing coating 105 of the carbon dioxide capture devices 115. It is desirable and advantageous for the carbon dioxide capture devices 115 to be arranged to optimize surface area of the coating substrate 110 within the interior space 120 of the encasement 125 of the algae farm 100, while still providing for such necessary atmospheric carbon dioxide (CO₂) 12, light (λ) 51 and water (H₂O) 52 to be present at the algae-containing coating 105 of the carbon dioxide capture devices 115. To this end, in one or more embodiments, coating substrate 110 of the carbon dioxide capture devices 115 is configured to allow spacing between the carbon dioxide capture devices 115 to be optimized for achieving desired surface area to volume requirements in regard to algae-coated substrate surfaces thereof. For example, in one specific embodiment, as shown in FIG. 6, the coating substrate 110 can be planar (i.e., generally flat) bodies that extend vertically in a spaced-apart arrangement. In one or more other embodiments, the coating substrates 110 may have a different physical configuration [e.g., contoured panels, cylindrical bodies, volumes of discrete elements (e.g., pellets, spheres, etc.), and the like] and may be arranged in a different spaced-apart manner (e.g., extend horizontally in a spaced-apart arrangement).

In preferred embodiments, the ability for carbon dioxide capture devices configured in accordance with the present invention (e.g., carbon dioxide capture devices 115) enables an advantageous level of surface area to volume in regard to algae-coated substrate surfaces thereof. In preferred embodiments, these advantageously high levels of coated surface area are achieved through the algae-containing coating 105 of the carbon dioxide capture devices 115 being relatively lightweight and thin (e.g., relative to a layer of ocean bound algae). In turn, this relatively lightweight and thin coating permits the coating substrate 110 to also be relatively lightweight and thin (e.g., a thin, durable, lightweight polymeric material such as a plastic sheet or panel). Together, such lightweight and thin nature of the algae-containing coating 105 and the coating substrate 110 provides for an advantageous level of surface area to volume in regard to algae-coated substrate surfaces.

As best seen in FIG. 6, the encasement 125 of the algae farm 100 serves to structurally support components (e.g., carbon dioxide capture devices 115) therein. In one or more embodiments, the encasement 125 can be a solid material that will partly or fully enclose the carbon dioxide capture devices 115. In other embodiments, the encasement 125 can be a skeletal or frame-type structure having openings in sidewalls thereof. The encasement 125 may have a retaining structure therein having a plurality of the carbon dioxide capture devices 115 engaged therewith (e.g., mounted thereon). The encasement 125 may be used for various purposes such as protection of the carbon dioxide capture devices 115 from the exterior environment, maintaining the interior environment of the algae farm 100 [e.g., water; humidity; nutrient(s); gas such as carbon dioxide or oxygen; temperature; light; etc.] within desired range(s) and/or allowing ease of movement and positioning of the algae farm 100 by moving the encasement and the interior algal farm components as unitary structure. Part or all of the encasement 125 (e.g., walls and/or ceiling) may permit the transmission of light therethrough (e.g., be transparent or translucent), as well as allow for exchange of gases (e.g., CO₂, O₂, etc.), and any other desired material (e.g., a nutrient) through the encasement in order to promote algae photosynthesis.

It is well-known that different types (e.g., species) of algae function optimally under different wavelengths and intensities of light, that life and function of algae is susceptible to certain intensities and wavelengths of light and that one or more other component(s) of a carbon dioxide capture device (e.g., besides the algae itself) may be damaged or otherwise adversely affected by certain intensities and wavelengths of light. For example, ultraviolent radiation “UV” may degrade a plastic component and/or injure or kill algae cells; while excessive light including photosynthetic wavelengths may injure or kill algae cells. The encasement 125 may reduce some wavelengths of light 51 and/or reduce light passage (e.g., be colored, be translucent, may comprise a UV absorber), for purposes such as to mimic shading for an algae that normally grows in a shaded environment, reduce UV light degradation of a component of the algae farm 100, and/or otherwise protect and improve the service life of the algae farm 100. In other embodiments, illumination of the carbon dioxide capture devices to optimize the wavelengths of light and total light intensity may be enhanced through features such as a reflective surface such as a mirror and/or an artificial lighting to increase photosynthesis, and such features may be attached to or be part of the interior parts of the encasement (not shown).

Light 51 (λ) present at the algae-containing coating 105 may be from a natural source (e.g., sunlight) and/or an artificial source (e.g., an electrically-powered lighting device). Light 51 is not limited to any particular wavelength or range of wavelengths, other than that needed to promote photosynthetic conversion of CO₂ by algae of algae-containing coating 105. In preferred embodiments, the light source is selected to promote or optimize photosynthetic conversion of CO₂ by algae of algae-containing coating 105. Similarly, water 52 (H₂O) present at the algae-containing coating 105 may in the form of natural ambient atmospheric moisture or more preferably may be in the form of an artificially-created water vapor (e.g., spraying droplets of water, flowing a stream of water, creating discrete pools of water and the like).

As can be seen from the disclosures herein, a carbon capture facility's function is to maximize the surface area such as relative to a given terrestrial surface area, preferably vertically but horizontally as well, through use of thin coatings (e.g., the algae-containing coating) and thin substrates (e.g., the coating substrate) comprising photosynthetic organism(s). Advantageously, carbon dioxide capture devices configured in accordance with embodiments of the present invention achieve a surface area to volume value for algae-based photosynthesis not able to be achieved in nature. The result of this is the capability to photosynthetically convert magnitudes of order greater amounts of atmospheric carbon dioxide on a unit volume basis than is possible in nature.

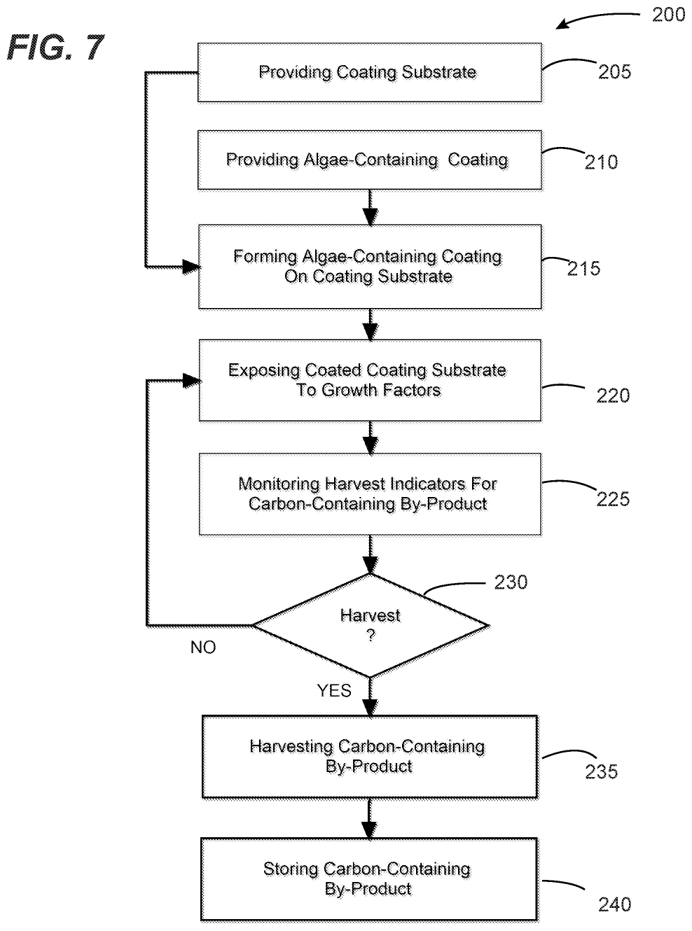

FIG. 7 discloses a method 200 in accordance with an embodiment of the present invention, which is adapted for providing capture of carbon dioxide from the air (e.g., atmospheric carbon dioxide). In preferred embodiments, the method 200 is adapted for operating a carbon capture facility (e.g., an algae farm 100 discussed above in reference to FIGS. 4-6) for capturing atmospheric carbon dioxide. To this end, the method 200 can employ carbon dioxide capture devices and apparatuses described in accordance with the disclosures herein. It is disclosed herein that the method 200 may similarly or identically used for capturing other greenhouse gases besides carbon dioxide (e.g. methane, nitrous oxide, hydrofluorocarbons, perfluorocarbons, sulfur hexafluoride).

The method 200 includes a plurality of operations that jointly provide for the aforementioned capture of carbon dioxide from a gas such as air. In a general sense, the method 200 provides for the structures and functionalities discussed above in reference to FIGS. 4-6. Examples of such functionalities include providing a carbon dioxide capture facility (i.e., system) components for enabling the photosynthetic conversion of carbon dioxide to a carbon-containing by-product, providing operating conditions that enable photosynthetic conversion of carbon dioxide to a carbon-containing by-product, harvesting carbon-containing by-product resulting from the photosynthetic conversion of carbon dioxide and/or implementing sequestration of the carbon-containing by-product.

As shown in FIG. 7, a first operation 205 of the method 200 is performed for providing a substrate for having a coating formed thereon (“a coating substrate”). The coating substrate can be any material capable of maintaining the coating thereon (e.g., such as the coating substrate 110 discussed herein upon which an algae-containing coating 105 is applied). Various coating substrates are described herein, but preferably the coating substrate is a lightweight material such as a plastic, particularly a thin plastic sheet. In one or more embodiments, the coating substrate includes a contoured portion (e.g., cylindrically-shaped portion. such as in the form of a tube or container). The coating substrate preferably has sufficient strength, stiffness and/or rigidity enable unsupported or supported mounting of the coating substrate in a horizontal or vertical arrangement, as well as various angles. It is disclosed herein that the coating substrate can comprise an article of manufacture retrieved from a waste stream, which is used as the coating substrate in an as-made manner, used in a modified manner as the coating substrate, or used as a raw material for making the coating substrate.

A second operation 210 of the method 200 is performed providing a coating composition having photosynthetic organism therein (i.e., a photosynthetic organism containing coating composition), which is to be used as a coating on the coating substrate. A photosynthetic organism containing coating composition in accordance with one of more embodiments of the present invention is preferably configured for being applied as a coating onto the coating substrate, for adhering to the coating substrate, and for providing photosynthetic conversion of at least one greenhouse gas (i.e., the photosynthetic organism of the photosynthetic organism containing coating composition is a living photosynthetic organism). Optionally, the photosynthetic organism containing coating composition (and/or the coating substrate) can be configured for enabling the photosynthetic organism containing coating to be removed (e.g., harvested) from the coating substrate. The photosynthetic organism containing coating is preferably formulated in a manner to exhibit sufficient solidity and adhesion to the coating substrate to remain a suitable surface layer for providing maximal surface area to conduct photosynthesis. The algae-containing coating 105 discussed herein is an example of the photosynthetic organism containing coating composition.

Providing a photosynthetic organism containing coating composition (e.g., containing algae or other photosynthetic organism) may be performed in a manner that includes obtaining living photosynthetic organism (e.g., algae). Such living photosynthetic organism can typically be grown by techniques described herein, as would be known to one of ordinary skill in the art or purchased from a commercial vendor. The living photosynthetic organism can be mixed with a material (e.g., a hydrophilic polymer and/or hydrogel material) that maintains enough of the cells of the photosynthetic organism alive for a period of time (e.g., minutes to years, days to weeks, etc.) to conduct photosynthetic conversion of carbon dioxide (and/or other greenhouse gas) into a non-gas carbon-containing by-product.

After the coating substrate and the photosynthetic organism containing coating composition have been provided, a third operation 215 of the method 200 is performed for forming a coating of the photosynthetic organism containing coating composition (e.g., an algae containing coating) onto one or more surfaces of the coating substrate. Forming the photosynthetic organism containing coating may be conducted by applying the photosynthetic organism containing coating to the coating substrate by any means known to those of ordinary skill in the art of general coating application (e.g., spraying, dipping, rolling, brushing, and the like). Formation of the photosynthetic organism containing coating may include texturizing an exposed surface of the coating to increase surface area of the coating exposed to ambient air. For example, a pattern of recesses can be formed within the coating, which are fully or partially the thickness of the coating, to create a coating having a 3-dimensional surface structure. Formation of the photosynthetic organism containing coating may include application of a primer or undercoat onto the coating substrate (or otherwise treating the coating substrate) prior applying the photosynthetic organism containing coating composition for affecting adhesion of the photosynthetic organism containing coating and/or activity of the photosynthetic organism itself. The photosynthetic organism containing coating is preferably formed in a manner to exhibit sufficient solidity and adhesion to the coating substrate to remain a suitable surface layer for providing maximal surface area to conduct photosynthesis. In preferred embodiments, forming the photosynthetic organism containing coating is performed in a manner that maintains living conditions of the photosynthetic organism (e.g., temperature, light, pressure, pH, etc.) within a tolerance so that a sufficient amount of the photosynthetic organism remains alive in the coating upon the coating substrate to conduct a desired level of photosynthesis.

After the photosynthetic organism containing coating is formed on the coating substrate, a fourth operation 220 of the method 200 is performed for exposing the photosynthetic organism containing coating to photosynthesis promoting factors (“growth factors”). In preferred embodiments, exposing the photosynthetic organism containing coating to the growth factors includes exposing the photosynthetic organism containing coating to environmental elements needed for photosynthesis (e.g., the exposure conditions 53 of carbon dioxide 12, water 52, and light 51 of a wavelengths that promotes photosynthesis described at FIGS. 5 and 6). The growth factors may also include nutrient(s) and appropriate environmental conditions (e.g., temperature, pressure, pH, etc.) suitable for both photosynthetic activity of the photosynthetic organism as well as to maintain the general metabolic health of the photosynthetic organism.

In response to exposing the photosynthetic organism containing coating to growth factors, the photosynthetic organism photosynthetically produces a carbon-containing by-product. As discussed above in reference to FIGS. 5-6, a carbon-containing by-product 49 produced in accordance with embodiments of the present invention can be in any number of forms (e.g., discrete volume of material on the photosynthetic organism containing coating and/or volume of material within the photosynthetic organism containing coating). The carbon-containing by-product can be sequestered in an environment wherein degradation of the carbon-containing by-product is minimalized and/or the carbon-containing by-product (and possibly the coating(s) used in a carbon dioxide capture farm (“carbon dioxide farm”)) is converted into another useful product. As discussed above in reference to FIGS. 5-6 in regard to the algae-containing coating 105, the carbon-containing by-product 49 crated from photosynthetic conversion of a greenhouse gas such as, for example, carbon dioxide can be in any number of forms.

A fifth operation 225 of the method 200 is performed for monitoring harvest indicators to determine when the carbon-containing by-product is ready for being harvested. In one or more embodiments, such monitoring is performed to determine when the carbon-containing by-product and/or the photosynthetic organism containing coating exhibit conditions indicating that harvesting of the carbon-containing by-product (and possibly the photosynthetic organism containing coating) is recommended. Examples of indicators that harvesting of the carbon-containing by-product (and possibly the photosynthetic organism containing coating) is recommended include, but are not limited to, expiration of a designated period of time from when the photosynthetic organism containing coating was formed; the photosynthetic organism containing coating exhibiting an attribute indicating a designated level of degradation in photosynthetic conversion; the photosynthetic organism containing coating, the substrate coating, and/or the carbon-containing by-product exhibiting an attribute (e.g., weight, staining property, density, color, etc.) determined to indicate harvesting is recommended; changes in the amount of materials needed for photosynthesis; changes in materials compositions resulting from photosynthetic conversion (e.g., the carbon-containing by-product and/or outgassing from the production thereof; and the like).

In one or more embodiments, monitoring harvest indicators can be performed through the use of a gas-monitoring device that detects the quantity of those materials associated with photosynthetic conversion (e.g., a reduction or loss of carbon dioxide and/or water uptake and/or reduction or loss of oxygen release, which are indicative of reduced photosynthesis and reduced production of a carbon-containing by-product). In one or more embodiments, monitoring harvest indicators can be performed by weighing the photosynthetic organism containing coating (or carbon dioxide capture device comprising same), as such weight will increase as photosynthesis produces the carbon-containing by-product, and a reduction or loss of measured increases in weight would be indicative of reduced photosynthesis and reduced production of a carbon-containing by-product. In one or more embodiments depending upon the carbon-containing by-product, monitoring harvest indicators can be performed through staining with a dye specific for the carbon-containing by-product (e.g., a stain for assessing production of cellulose, saccharide, and/or the like).

In response to monitoring the harvest indicators, a sixth operation 230 of the method 200 is performed for determining if the photosynthetic organism containing coating is to be harvested or is to be further exposed to the growth factors. After it is determined that the carbon-containing by-product is to be harvested, a seventh operation 235 of the method 200 is performed for harvesting the carbon-containing by-product. Harvesting the carbon-containing by-product may include delaminating the carbon-containing by-product from the photosynthetic organism containing coating if it is in the form of a layer of material upon the photosynthetic organism containing coating and/or may include extracting (e.g., solubilizing) the carbon-containing by-product with a solvent (e.g., water, glycerol, alcohol), and/or may include delaminating the photosynthetic organism containing coating from the coating substrate. In one or more embodiments, such delamination may be accomplished by changing temperature, by changing hydration of the carbon-containing by-product and/or photosynthetic organism containing coating, by applying physical force (e.g., shaking, sonicating, physical grasping with a device to pull a layer of material from another material, etc.), and/or by contacting with a chemical that changes adhesive properties (e.g., a solvent), such as in a manner described herein or as would be known to those of ordinary skill in the art.

Following harvesting of the carbon-containing by-product, an eighth operation 240 of the method 200 is performed for storing the carbon-containing by-product. Storing the carbon-containing by-product may be accomplished by placing the carbon-containing by-product (and possibly the photosynthetic organism-containing coating and coating substrate) in enhanced carbon sinks (e.g., underneath the surface of the earth or within the ocean), by conversion of captured carbon to useful solid or liquid material such as plastics, carbon fibers, cellulose-based building materials, biofuel, carbon-based chemicals (i.e., captured carbon utilization), etc.), by engaging in terrestrial and/or coastal blue carbon sequestration (e.g., fertilizing plants with the carbon-containing by-product) and/or by promoting carbon mineralization of the carbon-containing by-product. Preferably, such storing is performed in a manner that sequesters (i.e., provides for long-term storage) the carbon-containing by-product (e.g., storage in an enhanced carbon sink). Thus, such storing can include long-term sequestration and/or production of captured carbon utilization such as via useful product(s).

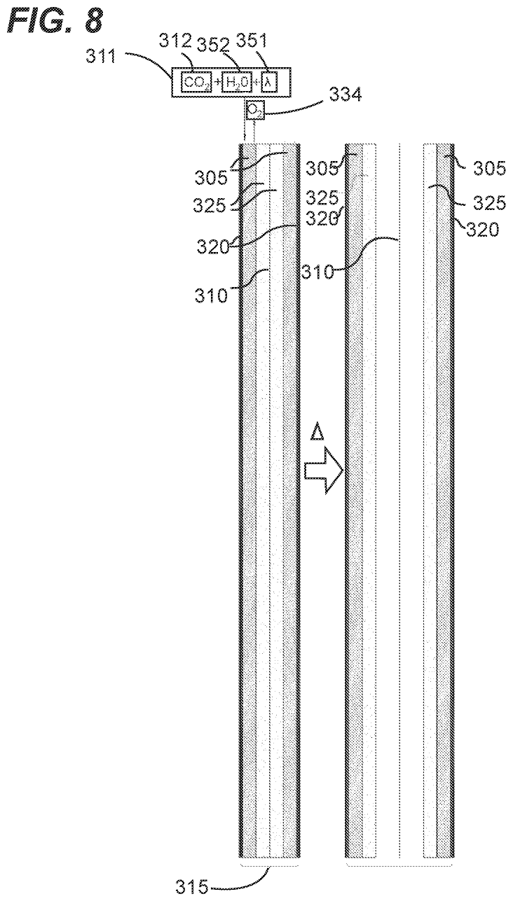

Referring now to FIG. 8, a photosynthetic organism containing coating delamination technique configured in accordance with an embodiment of the present invention. A photosynthetic organism containing coating 305 formed on a coating substrate 310 jointly provide a carbon capture device 315. As shown, through exposure to photosynthetic organism growth factors 311 of carbon dioxide (CO₂) 312, light (λ) 351 and water (H₂O) 352, the photosynthetic organism containing coating 305 releases oxygen (O₂) 334 and the carbon-containing by-product 320 formed thereon. Optionally, the carbon-containing by-product 320 can be within the photosynthetic organism containing coating 305 and/or extracted from within the photosynthetic organism containing coating 305. The photosynthetic organism containing coating delamination technique 300 is a technique for harvesting the carbon-containing by-product. In one or more embodiments, delamination of the photosynthetic organism containing coating 305 from the coating substrate 310 is performed by the application of heat (Δ) (though other methods may be used). The recovered carbon-containing by-product may then be placed into long-term sequestration and/or converted to other usable products.

The carbon dioxide capture device 315 may include an undercoat 325 (e.g., a layer thereof) between the photosynthetic organism containing coating 305 and the coating substrate 310. The undercoat 325 may be a polymeric material, a coating, a hydrogel, an adhesive, etc. In one or more embodiments, the undercoat may be a material capable of sustaining the life and photosynthetic activity of the photosynthetic organism of the photosynthetic organism-containing coating 305. In one or more embodiments, the undercoat 325 may be a layer of material that serves to enhance adhesion between the photosynthetic organism containing coating 305 and the coating substrate 310 and/or that enables selective delamination of the photosynthetic organism containing coating 305 from the coating substrate 310. In regard to enhancing adhesion between the photosynthetic organism containing coating 305 and the coating substrate 310, examples of the undercoat 325 includes, but are not limited to, a clear spray adhesive and/or polyvinyl alcohol, while polymeric material comprising a shear thinning property, such as xanthan gum, guar gum, or a combination thereof often have a viscosity suitable for application and/or adhesion to plastic surfaces (e.g., plastic containers, plastic bottles, etc.). The undercoat 325 may also function to supply water, humidity, and/or nutrient(s) to the photosynthetic organism containing coating 305, with a hydrogel comprising water being an example of such an undercoat. In other embodiments, adhesion of photosynthetic organism containing coating 305 may be promoted by techniques such as reduction in the initial water content (“hydration”) of the photosynthetic organism containing coating 305 and/or abrasion of the surface (e.g., mechanical abrasion, chemical abrasion, thermal damage, etc.) of the coating substrate 310 without the use of an undercoat.

As shown in FIG. 8, in at least one embodiment of the present invention, the carbon dioxide capture device 315 is disassembled by the undercoat 325 delaminating from the coating substrate 310 after application of heat (Δ). For example, a temperature-change responsive adhesive (e.g., a hot melt adhesive) used as an undercoat 325 may be delaminated by a change in temperature. In one or more embodiments, delamination may be accomplished by allowing the undercoat 325 to dry and lose moisture which reduces those undercoat's size and/or adhesive properties to the photosynthetic organism containing coating 305 and/or the coating substrate 310. In one or more embodiments, delamination of the photosynthetic organism containing coating 305, the layer of carbon-containing by-product 320, the undercoat 325 (if present), and/or the coating substrate 310 may be similarly accomplished depending upon the properties of the photosynthetic organism containing coating 305, the undercoat 325, and/or layer of carbon-containing by-product 320. Delamination may be preferable for ease of recovery of the coating substrate 310, the photosynthetic organism containing coating 305, and/or the carbon-containing by-product 320, as well as allowing reuse of the coating substrate 310 in creation of a carbon dioxide capture device with a new layer of photosynthetic organism containing coating.

In regard carbon dioxide capture devices disclosed herein, the type of carbon-containing by-product produced by such carbon dioxide capture devices and photosynthetic organism containing coatings thereof may be increased by selection of the specific photosynthetic organism (e.g., algae) used in the photosynthetic organism containing coatings and/or growth factors used during organism growth and photosynthesis. For example, it is well known that algae photosynthetically convert carbon dioxide into a sugar (i.e., a carbohydrate) such as ribitol and/or sorbitol. It is also well known that certain types of algae photosynthetically convert carbon dioxide into cellulose.

Carbon dioxide captured in accordance with embodiments of the present invention may be accounted for in a manner that generates a carbon credit. Such carbon credits can be utilized as an asset that is sold, traded or otherwise monetized (e.g., Green-e Center for Resource Solutions, 1012 Torney Ave., 2nd Floor, San Francisco, Calif. 94129). In one or more embodiments, such carbon credits are based upon an amount of carbon-containing by-product produced and/or an amount of post-product produced from the carbon-containing by-product. For example, a dried film comprising an algae-containing coating and/or a carbon-containing by-product produced by the algae-containing coating may be converted (e.g., mechanically ground) into a granular material suitable for long term carbon sequestration and/or admixed with a other materials [e.g., a polymeric material to produce other materials (e.g., a plastic)], with the other materials created by altering or combining the carbon-containing by-product being an example of a post-product produced form the carbon-containing by-product.

In one or more embodiments, a carbon dioxide capture device or material comprises an artificial lichen (“pseudolichen”). A lichen is a symbiotic relationship between a fungus, known as a mycobiont, and a photosynthetic organism, known as a photobiont, such as an alga (“phycobiont”) (e.g., a green alga) and/or a cyanobacterium (“cyanobiont”). The mycobiont provides structural support of the photobiont's cells as a layer for efficient photosynthesis while providing protection from the environment. In one or more embodiments, the mycobiont can be partly or fully replaced by a non-fungal material and is known herein as a pseudomycobiont.

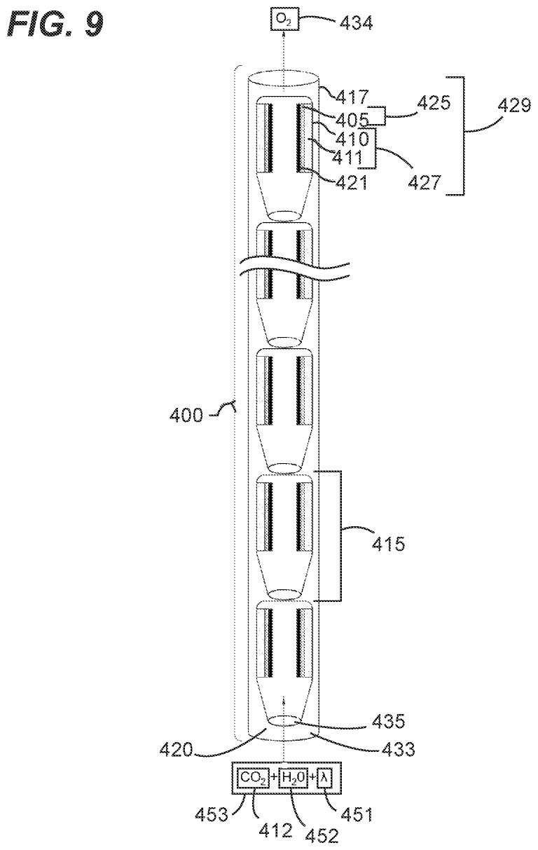

Referring now to FIG. 9, a carbon dioxide capture apparatus 400 configured in accordance with one or more embodiments of the present invention is shown. The carbon dioxide capture apparatus 400 is characterized by a plurality of discrete carbon dioxide capture devices 415 that are retained within a device retaining structure 417 such as by being stacked in an end-to-end fashion within an interior space of the device retaining structure 417. Each of the carbon dioxide capture devices 415 includes a substrate 410 having one or more surface areas with an algae-containing coating 405 formed thereon (i.e., the “coating substrate 410”). Optionally, the carbon capture devices 415 comprise an undercoat 411 between the substrate 410 and the algae-containing coating 405. Preferably, the coating substrates are plastic containers such as single-use beverage containers recycled from a waste stream. The device retaining structure 417 can be a sleeve or tube made from a material such as, for example, glass or plastic. For enabling light transmission, the respective materials from which the coating substrate 410 and the device retaining structure 417 are made are light transmissive (e.g., fully or partially transparent). For enabling water vapor and air transmission, the coating substrate 410 and the device retaining structure 417 each have one or more openings 433 therein for enabling airflow therethrough. In certain embodiments, a device restraining structure may function as an encasement, though one or more additional encasement(s) may be part of a carbon dioxide capture facility depending upon the size and configuration of the carbon dioxide capture facility.

As depicted in FIG. 9, the an algae-containing coating 405 configured in accordance with one or more embodiments of the present invention comprises the photobiont 425 (e.g., algae), while the part of the algae-containing coating that is not the algae [e.g., material capable of sustaining the life and photosynthetic activity of the organism part of the algae-containing coating (“the life supporting material”)], the coating substrate 410 and/or undercoat 411 is the pseudomycobiont 427. In some embodiments, one or more components of the pseudomycobiont 427 (e.g., the life supporting material, the coating substrate, the undercoat) may comprise an organism such as a fungus (e.g., a lichen derived fungus). The combination of the photobiont 425 and pseudomycobiont 427 is the pseudolichen 429.

Preferably, the coating substrate may be a transparent container that would normally have been discarded into trash, such as a bottle for soda, water, or other material, that is repurposed in the embodiments herein. In one or more embodiments, a single pseudolichen may be referred to as a pseudoleaf, or referred to depending upon the type of coating substrate used, such as a container being used as the coating substrate and the carbon dioxide capture device accordingly created referred to as a leaf-container or pseudoleaf container, a bottle being used coating substrate and the carbon dioxide capture device created being referred to as a leaf-bottle or pseudoleaf bottle, etc. As some lichen grow on or within rocks such as gypsum, and in some embodiments a gypsum (e.g., powdered or granular gypsum added as a pigment) and/or other light translucent/reflective material may be incorporated in the algae-containing coating, undercoat, coating substrate, or a combination thereof. A pseudolichen may comprise a vivarium for a photobiont, such as a sealable container wherein the photobiont is placed along with other components of the pseudomycobiont (e.g., a polymeric material used as the life supporting material, an undercoat, and/or a hydration layer, etc.).

As shown in FIG. 9, openings(s) 433 at both ends of the cylindrical encasement 417 allows carbon dioxide (CO₂) 412, water (H₂O) (e.g., water vapor) 452, and wavelengths of light (λ) 451 (i.e., exposure conditions 453), that promote photosynthesis by the algae in the algae-containing coating 405 to enter the spaces 420 between the encasement sleeve 417 and the carbon dioxide capture devices 415, as well as enter openings(s) of 435 (e.g., lack of a cap) of the carbon dioxide capture device(s) 415. A carbon-containing by-product layer 421 is produced by the photosynthetic activity of the algae in the algae-containing coating 405, and oxygen 434 released which leaves the encasement at the upper end of the carbon dioxide capture apparatus 400. The encasement opening(s) 433 and/or a carbon dioxide capture device's opening(s) 435 may also be in any form or location that allows gas and water exchange into and/or out of the encasement 417 and/or a carbon dioxide capture device 415 such as pores, holes, removal of a cap or other sealing device, the presence of a gas permeable membrane, etc.

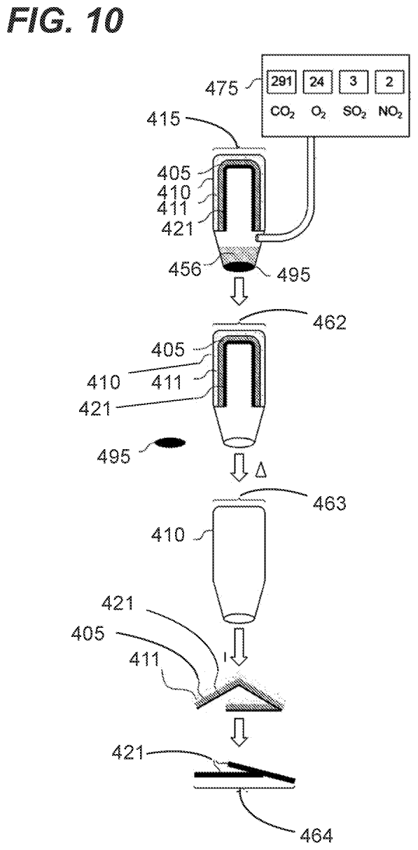

FIG. 10 shows a process for implementing harvesting of carbon-containing by-product for a carbon dioxide capture device of the carbon dioxide capture apparatus as shown in FIG. 9. The carbon dioxide capture device 415 wherein the coating substrate 410 is a plastic beverage bottle comprising an algae-containing coating 405, an undercoat 411, and a carbon-containing by-product layer 421 produced by the photosynthetic activity of the algae present in the algae-containing coating 405. The carbon dioxide capture device 415 is sealed via a cap 495 to entrap and maintain a hydration layer 456 (e.g., a pool of water, a hydrogel comprising such water) to supply water, humidity and/or nutrients to the algae-containing coating 405 and/or undercoat 411. A gas content detector and/or gas exchange device (“gas device”) 475 [e.g., a device that measures, supplies and/or removes one or more gas(s)] is operatively associated with the carbon dioxide capture device 415. The gas device 475 in this instance comprises a gas content detector that in this instance measures the amount of carbon dioxide, oxygen, sulfur dioxide, and nitrogen dioxide content as those gases may be uptaken (e.g., captured) and/or released depending upon the organism(s) selected for inclusion in the algae-containing coating 405.

A gas device may be similarly used to measure and/or alter the amount of one or more gases (e.g., water vapor, carbon dioxide, oxygen, etc.) in a carbon dioxide farm, a stacked container tree, a carbon dioxide farm container, an encasement, a carbon dioxide capture device, etc. or a combination thereof, and/or to measure the amount of carbon capture occurring through metabolic activity of living cells (e.g., photosynthetic cells). For example, a gas (e.g., carbon dioxide, air) may be actively injected (“flow”) (e.g., from a compressed gas cylinder) into a stacked container tree, and various gas devices such as a variable area gas flowmeter may be used to control of the amount of gas flow of and/or measurement of a gas's content in the carbon capture devices within the stacked container tree. Such gas cylinders, gas measurement and flow control devices are known in the art and commercially available [Verner Software & Technology, 13979 SW Millikan Way, Beaverton, Oreg. 97005 (“Verner Software & Technology”); Dwyer® 102 Indiana Hwy. 212, P.O. Box 373 Michigan City, Ind., 46360 (“Dwyer”); Southern Gas And Supply, 1512 N. Main St., Hattiesburg, Miss. 39401].

As shown in FIG. 10, at depiction 462, the carbon dioxide capture device is modified to have the cap 495 and the hydration layer (not shown) removed for such purpose as to allow ease of recovery of the carbon-containing by-product 421, algae-containing coating 405, and undercoat 411 by delamination from the bottle coating substrate 410 (shown in depiction 463). In this instance delamination is induced by a temperature change by applied heat (Δ) to the carbon dioxide capture device of depiction 462 leaving an empty bottle coating substrate 410 separated from the carbon-containing by-product 421, algae-containing coating 405, and undercoat 411 (depiction 463). The carbon-containing by-product 421 is subsequently separated from the algae-containing coating and undercoat (not shown) in depiction 464 for commercial use in a product and/or long-term carbon sequestration.

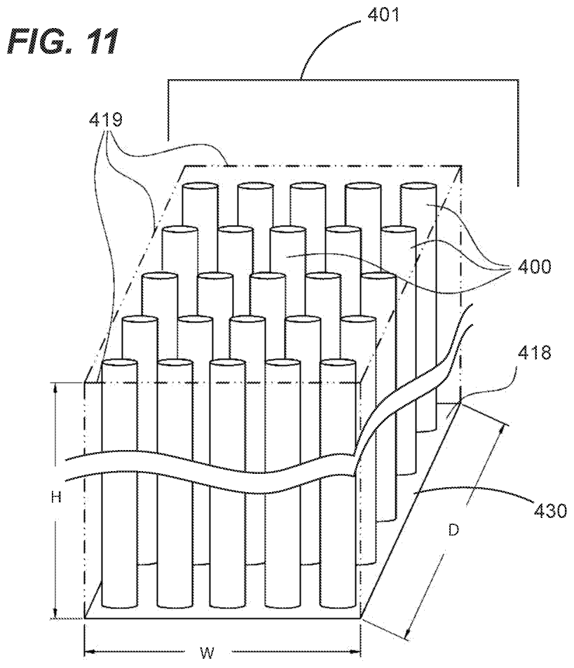

As depicted in FIG. 11, a plurality of the carbon dioxide capture apparatuses 400 may be arranged in an adjacent manner to form an algae farm 401 (i.e., a carbon dioxide capture farm based on photosynthesis by algae). In one or more embodiments, each of the carbon dioxide capture apparatuses 400 may be held into place by one or more connective support(s) 418 (e.g., a mesh, wires, strings, a grid, a platform, etc.). The vertical and horizontal area (height, depth, and width designated “H,” “D,” and “W,” respectively) of the algae farm 401 may be increased as desired in any of the vertical and horizontal directions. For example, the plurality of cylindrical carbon dioxide capture apparatuses 400 are depicted in FIG. 10 as organized to occupy an approximate cubical area, and the carbon dioxide capture apparatuses 400 and/or connective supports 418 may provide a mechanism for ease of movement and orientation carbon dioxide capture of multiple smaller carbon dioxide capture apparatuses 400 as a single movable unit known herein as a carbon dioxide farm container 419. In preferred embodiments, a carbon dioxide farm container is approximately 1 cubic meter for ease of calculating the amount of carbon dioxide capture in a carbon dioxide farm that may comprise a plurality of carbon dioxide capture apparatuses and/or carbon dioxide farm containers. Though 25 such carbon dioxide capture apparatuses 400 are depicted, less or more (e.g., 2 to thousands, millions or more) may be included in the algae farm 401 depending upon the size of each carbon dioxide capture apparatus 400 and the desired configuration of the carbon dioxide capture apparatuses 400.

One or more attachment(s) (“orientation attachment”) 430 may be operatively connected to the connective support(s) 418, carbon dioxide capture apparatus(s) 400, larger algae farm 401, and/or carbon dioxide farm container 419 for ease of movement (e.g., transport, stacking, etc.) and/or orientation of the connective support(s) 418, carbon dioxide capture apparatus(s) 400, larger algae farm 401, and/or carbon dioxide farm container 419. Examples of an orientation attachment 430 include a handle for manual orientation, a connection to a motor for mechanized orientation, etc., and any other orientation attachment as known to those in the art that may be used. The connective support(s) 418, carbon dioxide capture apparatus(s) 400, larger algae farm 144, and/or carbon dioxide farm container 419 may be orientated at any angle from 0 degrees to 360 degrees in any direction in three dimensions (e.g., vertically, horizontally) as desired, generally to optimize lighting, gas exchange, hydration, etc. for photosynthesis.

In many embodiments, a plurality of carbon dioxide farm containers may be stacked on top of each other (such stacks referred to herein as “trees”). Preferably, a carbon dioxide farm may comprise 2 to thousands or millions of stacked containers which may be held in place additional physical supports as desired (e.g., stacked containers 10 stories high, 30 meters high, etc.). For example, a carbon dioxide farm of several meters wide, long and tall may be used within a personal dwelling or yard of house. A carbon dioxide farm may cover a surface area such as 1 to about 100,000 feet or meters long and 1 to about 100,000 feet or meters wide (e.g., carbon dioxide farms that are square mile(s) or square kilometer(s) in size). An exemplary carbon dioxide farm would comprise a plurality of vertically stacked container trees (e.g., each tree about 30 meters high) encompassing a square kilometer of horizontal surface area.

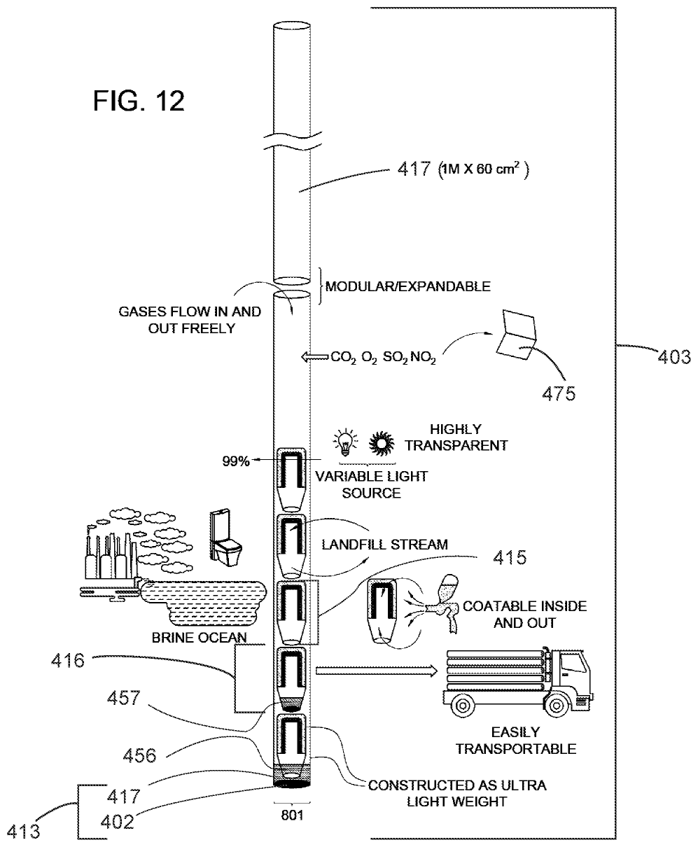

FIG. 12 depicts many of the features of a carbon sequestration system 403 described herein, using transparent plastic sleeve(s) (e.g., 1 M by 60 cm²) 417 with a cap 402 as encasement(s) 413 for a plurality of carbon dioxide capture devices 415 created from plastic bottle coating substrates previously destined disposal in a landfill. The plastic bottle coating substrates are obtained from the landfill waste disposal stream (“landfill stream”) and after use in carbon dioxide capture devices may be returned to the landfill stream for long-term sequestration of the carbon dioxide now contained within the devices' carbon-containing by-product(s) or subjected to one or more implementations of captured carbon utilization. Both the carbon dioxide capture devices 415 and the encasement sleeves 417 are constructed from ultra-light weight materials and are easily transportable to a location for use as a carbon dioxide farm, long term sequestration of captured carbon dioxide, and/or commercial use of the carbon-containing by-product. The encasement sleeve 417 is capped 402 on one end to retain a hydration layer 456 to supply hydration and/or nutrient to all uncapped carbon dioxide capture devices within the encasement sleeve. Similarly, a capped carbon dioxide capture device 416 is depicted containing an independent hydration layer 157 as an alternative embodiment. The hydration layer may comprise potable water and/or greywater (e.g., as depicted wastewater from toilets, industry, ocean brine, etc.). In some embodiments, the hydration layer, algae-containing coating, undercoat, and/or coating substrate may have little or no nitrogen content to foster saccharide production by an organism (e.g., a cyanobacteria). A carbon dioxide capture device 415 may be coatable (e.g., coatable by a carbon-capture coating, coatable by an undercoat, etc.) on two or more sides of a coating substrate (e.g., on internal surfaces and/or external surfaces of a bottle). Often both the encasement sleeve(s) and the carbon dioxide capture device(s) are highly transparent (e.g., 99% transparent to photosynthesis promoting wavelengths of light) provided by variable (“differing”) light source(s) (e.g., a natural light source such as the sun, an artificial light source such as a light bulb, etc.). The encasement sleeve(s) 417 may be operatively connected to gas device 475 (e.g., for gases such as H₂O, CO₂, SO₂, NO₂, etc.) particularly when the carbon dioxide capture device(s) 415 are unsealed (e.g., uncapped) and the gases freely flowing in and out through a gas permeable opening 433 in the encasement sleeve(s) 417. The encasement sleeve(s) and carbon dioxide capture device(s) are often modular and/or expandable such as by vertical stacking as well as horizontal placement in an area selected for an algae-farm (not shown).

In one or more embodiments, the coating substrate (e.g., a wall, a floor, a roof, a ceiling, a piece of furniture such as a table, a vehicle's surface, a plastic sheet, a metal surface, etc.) may not encased in an encasement or sealed. The coating substrate may also be partly or fully sealable (e.g., a soda bottle that has a cap screwed on; a carboy) to retain moisture, humidity, a nutrient, a gas (e.g., carbon dioxide, oxygen, carbon monoxide, water vapor), to promote a desired photosynthesis rate and/or duration of the photosynthetic organism.

In general aspects, the temperature of the photosynthetic organism may be maintained in a desired range use of natural and/or artificial heating and/or cooling source(s) (e.g., a greenhouse, electric heaters, air conditioners, fires, light reflective materials directing light toward the photosynthetic organism containing coating or material, etc.). A hydration layer (e.g., which may be an undercoat), a nutrient, a gas, one or more components of the photosynthetic organism containing coating, an undercoat, a coating substrate, an encasement, or a combination thereof, may be continuously or intermittently put into the carbon dioxide capture device and/or encasement to maintain or enhance photosynthesis. For example, a fresh layer of photobiont embedded in a pseudomycobiont may be placed on the previous layer of photobiont embedded in a pseudomycobiont. In another example, some or all of the components of the pseudomycobiont may be removed and replaced with fresh pseudomycobiont to maintain photosynthesis at a desired rate as the original photobiont cells die and/or as water, a nutrient, etc. become depleted.

It is contemplated herein that any photosynthetic (e.g., photobiont) organism may be used in a carbon dioxide capture device or material. Examples of a photosynthetic organisms include organisms that grow in oxygenic environments, such as photosynthetic cyanobacteria; organisms that grow in anoxygenic environments, such as green and purple bacteria (e.g., Chromatiaceae spp., Ectothiorhodospiraceae spp.), filamentous anoxygenic phototrophs, and the like; as well as phototrophic acidobacteria and phototrophic heliobacteria.

In one or more embodiments, a photosynthetic organism may comprise a genetically modified organism, particularly when a desired carbon-containing by-product is produced due to the genetic modification. For example, extacellular type-I cellulose has been produced by genetically modified Synechococus sp. PCC 7002 containing cellulose expression genes from Gluconacetobacter xylinus (Zhao et al., Cell Discovery, 1:1-12, 2015); and genetic modification of a photosynthetic organism may be used to convert carbon dioxide into a particular biomolecule, such as cellulose that is more likely to resist environmental (e.g., biological) degradation back into carbon dioxide. In another example, U.S. Pat. No. 10,131,870 discloses targeted genetic modification of Spirulina (which is synonymous with Arthrospira) may be conducted on Athrospiria platensis NIES-39 or Arthrospira sp. PCC 8005; as well as A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. consstricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In another example, U.S. Patent publication no. 2008/0113413 uses Synechococcus leopoliensis UTCC 100 (also known as Synechococcus elongatus PCC 7942) that was genetically modified to include Acetobacter xylinum NQ5's cellulose synthase operon for enhanced cellulose production. A photosynthetic organism may comprise putative gene(s) for production of a particular biomolecule, such as Spirulina spp. (e.g., S. platensis, S. maxima) possessing putative cellulase synthase genes, wherein production of the particular biomolecule may be induced by selected growth conditions and/or genetic modification.

In certain embodiments, a photobiont may be obtained from a lichen for use herein based the ability to survive a particular environment that the pseudolichen will function. For example, Ramalina maciformis lives in a dry desert state, and lichen such as Chrysothrix spp. (e.g., Chrysothrix chlorina) and Lepraria spp (e.g., Lepraria lobificans) survive using air humidity as a water source. Some lichens are adapted for extreme terrestrial cold environments such as Lecidea spp. (e.g., Lecidea laboriosa) and Pseudephebe spp. (e.g., Pseudephebe pubescens). Some lichen use a nitrogen source to grow, such as the Xanthoria spp. (e.g., Xanthoria polycarpa) and Physcia spp. (e.g., Physcia millegrana). Other lichen grow in temperate to subtropical climates such as Trentepohlia spp. (e.g., Trentepohlia aurea). However, a photosynthetic cyanobacterium and/or alga, particularly a unicellular, oligocellular and/or multicellular organism that preferably grows at a microscopic size that is not derived from a lichen may be used as a photobiont in a pseudolichen herein. Examples of a photosynthetic alga are any described herein or would be known to those of ordinary skill in the art in light of the present disclosures, such as Chlamydomonas reinhardtii, Chlorella sorokiniana UTEX1230 (Krujatz et al., Eng. Life Sci. 15:678-688, 2015), Dunaliella tertiolecta (Grizeau et al., Biotechnol. Lett. 8:261-264, 1986); Spirulina platensis, Spirulina maxima (Rao et al., World J. Microbiol. Biotechnol. 15:465-469, 1999); Synechococcos ssp. (May et al., J. Appl. Phycol. 19:181-183, 2007); Synechocystis sp. ATCC 27184; Synechococcus sp. UTCC 100; Gloeocapsa sp. UTEX L795; Oscillatoria princeps; Oscillatoria sp. UTEX L2435; Phormidium autumnale; Crinalium epipsammum; Anabaena sp.; Nostoc punctiforme; Nostoc muscorum UTEX 2209; Nostoc muscorum UTEX 1037; and Scytonema hofmanni. The photobiont or any other organism used in the pseudolichen may be an extremophile, such as an alkalphile, or a chemosynthetic organism that grows in low or no light, such as in a building's interior.

In one or more embodiments, a photosynthetic organism (e.g., a photobiont) may be combined with and/or replaced with a non-photosynthetic organism (e.g., a bacteria) that produces a carbon-containing by-product. Such a non-photosynthetic organism is known herein as a “non-photobiont.” For example, a cyanobacterium that converts carbon dioxide into a sugar such as glucose may be combined in the same layer as the non-photosynthetic organism, or separated into different layers or materials where the sugar can diffuse to the non-photosynthetic organism to be converted by the non-photosynthetic organism into another desired material such as bacteria produced cellulose. A non-photobiont may be combined (e.g., in and/or upon) a material capable of sustaining the life and activity of the non-photobiont, and such material may be the same or different as a life supporting material used for a photosynthetic organism as described herein. Such a combination of a non-photobiont and a material capable of sustaining the life and activity of the non-photobiont would be referred to herein as a coating when applied on a substrate as a layer (e.g., an undercoat applied to a coating substrate; a topcoat applied to a layer of photosynthetic organism coating which is a layer upon an undercoat that coats a plastic coating substrate, etc.). Examples of non-photobionts are known in the art, and include species from the genera Gluconacetobacter, Alacaligenes, Aerobacter, Achromobacter, Sarcina, Agrobacterium, Pseudomonas, Azotobacter, Rhizobium, and Acetobacter (Brown et al. J. Applied Polymer Science Appl. Polymer Symp. 37:33-78, 1983). Examples of non-photobionts used to produce cellulose commercially include Gluconacetobacter xylinus (e.g., ATCC 11142; ATCC 700178) and Gluconacetobacter europaeus (Yamada et al., Biosci, Biotechnol, Biochem 61 (8): 1244-1251, 1997; Leitao et al., Materials 6, 1956-1966, 2013). Another example of a non-photobiont includes bacteria such as Azotobacter vinelandii and Clostridium ljungdahlii that are capable of converting carbon monoxide in the presence of carbon dioxide and hydrogen into low molecular weight hydrocarbons such as ethanol (Canter, N. Tribology & Lubrication Technology, 73(4):14-15, 2017). U.S. Pat. No. 4,950,597 discloses cellulose produced by prokaryotic organisms such as Salmonella, Agrobacterium, and Rhizobium; and cyanobacterium such as Nostoc, Scytonema Anabaena or Acetobacter sp. use in wound bandages and implanted medical devices. In another example, U.S. Pat. No. 4,954,439 discloses microorganisms capable of extruding cellulose in ribbons that repeatedly reverse directions of extrusion, wherein such microorganisms include genus Acetobacter such as Acetobacter xylinum (e.g., strains NQ5; H1A; H1B; H1C; H2A; H2B; H5C; H5D; H6C; H8C; H8G; H14B; H15A; and H15B). U.S. Pat. No. 4,950,597 discloses methods for identifying cellulose II producing organisms, particularly among the species Acetobacter xylinum, Acetobacter hansenii, Acetobacter pasteurianus or Acetobacter aceti. U.S. Pat. No. 4,891,317 discloses altered cellulose production by Acetobacter xylinum, American Type Culture Collection (ATCC) No. 23769; while U.S. Pat. No. 4,942,128 discloses cellulose production by Acetobacter xylinum ATCC no. 23769 or ATCC no. 53582 (strain NQ5) in the presence of carboxymethylcellulose.

In additional examples of carbon-containing by-product producing organisms and use of such by-products, U.S. Pat. No. 7,803,601 discloses saccharide production (e.g., monosaccharides, oligosaccharides, polysaccharides, disaccharides) by cyanobacteria such as Synechococcus sp. PCC 7002, Synechococcus leopoliensis strain UTCC100, Agmenellum quadruplicatum UTEX B2268, and Synechococcus sp. ATCC 27264, as well as production of an extracellular sheath by these cyanobacteria. Extracellular sheaths produced by cyanobacteria are a carbon-containing by-product typically comprise carbon containing polymers such as polysaccharide(s), protein(s), and nucleic acid(s) [Stuart et al., SIME J., 10(5):1240-1251, 2016]. U.S. Pat. No. 7,803,601 discloses these cyanobacteria may comprise part of a bacterial, fungal, algal, and/or plant cellulose operon (e.g., acsAB gene operon from Acetobacter xylinum under a lac promoter in Synechococcus leopoliensis strain UTCC100) in order to produce cellulose (e.g., bacterial cellulose, cellulose I, cellulose II, crystalline cellulose, non-crystalline cellulose), or enhance glucose production under acidic conditions, in order to sequester carbon to create carbon credit units. U.S. Pat. No. 7,803,601 also discloses that the cyanobacteria may function in hypersaline conditions so that various water sources (e.g., brine) and environments (e.g., non-agricultural areas) may be used, and low or no nitrogen sources supplied to the cyanobacteria as cyanobacteria can fix nitrogen. U.S. Pat. No. 7,803,601 also discloses other commercial carbon containing by-products such a pharmaceuticals, chemicals, fatty acids, acylglycerols may be produced by the cyanobacterial, as well as equipment and methods for harvesting carbon-containing by-products. U.S. Pat. No. 7,803,601 further describes cellulose producing Acetobacter xylinum (e.g., Acetobacter xylinum NQ5) as capable of metabolizing up to 50% of extracellular saccharide (i.e., glucose) into a cellulose containing extracellular pellicle. In another example, U.S. Patent publication no. 2008/0085536 uses Agmenellum quadruplicatum UTEX B2268, Synechococcus sp. PCC 7002 and Synechococcus sp. ATCC 27264 for cellulose production in saline conditions. The carbon-containing by-product such as cellulose, protein, etc. produced by the photosynthetic organism and/or non-photobiont containing coating, carbon dioxide capture device, and/or material may be partly or fully isolated (see, for example, Zeng et al., Cellulose 21(6): 4455-4469, 2014) and used in various commercial products. Alga protein and carbohydrates can be incorporated into foam and plastics (Algix, 5168 Water Tower Rd., Meridian, Miss. 39301). A non-algal photosynthetic organism may be used instead of and/or in combination with an algal organism (e.g., a cyanobacterium) in combination with life supporting material, and such a non-algal organism may be selected based on the environment a carbon dioxide capture device will be used and/or the carbon-containing by-product that the organism produces. For example, a cyanobacterium will generally produce alcohol (e.g., a polyol such as glycerol), a sugar (e.g., glucose) and/or a polymer of alcohol(s) and/or sugar(s) such as cellulose. Microorganism produced cellulose has use in various commercial materials alone or in combination with other materials [e.g., a polymer such as poly(vinyl alcohol); Shi et al., Nanoscale, 6:970-977, 2014; Rebelo et al., Science and Technology of Advanced Materials, 19(1), 203-211, 2018] for uses such as electron (e.g., optical) applications such as transparent films and organic light emitting diodes (Wicklein, B. and Salazar-Alvarez, G., Journal of Materials Chemistry A 1(18):5469-5478, 2013; Ummartyotin et al., Ind Crop Prod 35(1):92-97, 2012) as well as batteries (Petersen et al., Appl Microbiol Biotechnol. 91:1277-1286, 2011; Feng et al., Carbohydr Polym. 87:644-649, 2012); a liquid absorbent material (e.g., oil, water; see for example Jin et al., Langmuir 27(5):1930-1934, 2011; Nata et al., RSC Advances 1(4):625-631, 2011; Sai et al., Journal of Materials Chemistry A 1(27):7963-7970, 2013; Ul-Islam et al., Carbohydr Polym 88(2):596-603, 2012); in biomedical applications such as patches for wounds (Fu et al., Carbohydr Polym 92(2):1432-1442, 2013; Ul-Islam et al., Carbohydr Polym 89(4):1189-1197, 2012) and in tissue engineering (Huang et al., Cellulose, 21:1-30, 2013; Andrade et al. J Bioact Compatible Polym 28(1):97-112, 2013; Saska et al., J Mater Chem 22(41):22102-22112, 2012; Svensson et al., Biomaterials 26(4):419-431, 2005); and food products (Kalia et al. Cellulose Fibers: Bio- and Nano-Polymer Composites., (2011) Springer, Berlin; Shi et al., Food Hydrocolloids 35:539-545, 2014; Chunshom et al., Journal of Science: Advanced Materials and Devices 3:296-302, 2018). U.S. Pat. No. 4,378,431 uses a Gram-negative bacterium, Acetobacter xylinum, to produce microcellulose on fiber such as polyester fiber. U.S. Pat. Nos. 9,090,713 and 9,670,289 irradiates microbial cellulose synthesized by Acetobacter xylinum (Gluconacetobacter xylinus) for medical implant biomaterials. Also, a photosynthetic organism generally releases oxygen during photosynthesis which may be captured and used for commercial or environmental (e.g., indoor or outdoor air quality improvement) applications.

Examples of sources of cells such as a photosynthetic organism, a mycobiont, and/or a non-photobiont organism are known in the art, such as the Collection of Algae at Goettingen University (Gottingen, Germany); and The Culture Collection of Algae at the University of Texas [(“UTEX”), Austin, Tex., U.S.A.). Such cells may be grown by any technique known to those of ordinary skill in the art, including small scale production suitable for a carbon dioxide farm in or next to a personal dwelling, to large scale [e.g., square mile(s)] carbon dioxide farms; and also may be obtained from commercial alga and microorganism production companies (Algae Research and Supply, 1405 Buena Vista Way, Carlsbad, Calif. 92008; Raw Living Spirulina, 6885 57th St, Vero Beach, Fla.; UTEX Culture Collection of Algae, 205 W 24th St, Biological Labs 218, The University of Texas at Austin, Austin, Tex. 78712; AlgEternal Technologies, LLC, 3637 TX-71, La Grange, Tex. 78945).

It is disclosed herein that one or more components of the carbon dioxide capture device or material may comprise an enzymatic or peptide additive, such as a hydrolytic enzyme (e.g., protease, lipase, amylase) to degrade one or more biomolecules that could be used as a nutrient for a living cell in the carbon-capture device (e.g., a photobiont cell), an antimicrobial peptide to retard the growth of an undesirable contaminating cell in the carbon-capture device (e.g., a photosynthetic organism coating), or a combination thereof.

Sources of polymeric materials (e.g., a coating, a plastic film, a plastic sheet, a hydrogel, an adhesive, a sealant, etc.) and polymeric material components are known to those of ordinary skill in the art (see, for example, U.S. patent application Ser. No. 12/696,651), though in certain embodiments polymers and other coating components may be obtained by recycling unused paint (e.g., unsold, partly used cans of paint). Examples of companies that recycle paint include Loop Recycled Paint (940 Chippawa Creek Rd, Niagara Falls, ON L2R 5T8, Canada) and Colortech ECO Paints Ltd. (411 East Main St, Suite B-4, Welland, Ontario L3B 3X3, Canada).

Cells used in the embodiments herein may be assayed for a desired level of living activity (“cell viability”) prior to and/or after incorporation in a coating, material, pseudolichen, carbon dioxide capture device, carbon dioxide farm, etc.; and components of the coating, material, pseudolichen, carbon dioxide capture device, carbon dioxide farm, etc.; may be replaced and/or augmented with additional materials (e.g., nutrients, fresh living cells) to maintain, for example, a desired level of photosynthetic carbon dioxide capture. Assays and equipment for cell viability are known to those of ordinary skill in the art, and examples include spreading a volume containing cells on a nutrient medium and counting cell colonies that grow (“plating”), or using a fluorimeter and/or microscope (e.g., a hand-held monitoring pulse-amplitude-modulation (PAM) fluorimeter, Heinz Walz GmbH, Eichenring 6, 91090 Effeltrich, Germany; fluorimeters available at PP Systems International, Inc., 110 Haverhill Road, Suite 301, Amesbury, Mass. 01913 U.S.A.) or to measure the presence of photosynthetic pigments in a photobiont (Schulze et al. BMC Biotechnology, 11:118, 2011). Other assays include cellulose fluorescing dyes (e.g., Pontamine Fast Scarlet 4B; SYTOX Green) as seen using a confocal microscope (Anderson et al. Plant Physiology, 152:787-796, 2010; Thomas et al; Journal of Microscopy, 00(0):1-15, 2017; Sato et al., Microbiol Cult Coll, 20(2):53-59, 2004), flow cytometric analysis (Lee et al., Biotechnology Letters, 22:1833-1838, 2000), or autofluorescence (Schulze et al., BMC Biotechnology, 11:118, 2011)

In addition to the sources described herein for a reagent, a living cell, it is disclosed herein that a coating substrate such as a physical support for a polymeric material (e.g., a coating, a hydrogel, an adhesive), an encasement, a connective support, an orientation attachment, gas control and measurement equipment, etc., and such a material, equipment, and/or a chemical formula thereof may be obtained from convenient source such as a public database, a biological depository, and/or a commercial vendor. For example, microorganism grow medium, growth container(s), physical support(s), gas measurement and control equipment, tubing, plug(s), filter(s), sanitizer(s) [e.g., bactericide(s), fungicide(s), etc.], plastic carboy fermenter(s), etc. may be obtained from commercial vendors such as Dwyer®; Verner; VWR International, Radnor Corporate Center Building One, Suite 200, 100 Matsonford Road, Radnor, Pa. 19087; Five Star Chemicals & Supply, Inc. 4915 E 52^{nd} Ave Commerce City, Colo. 80022; Bac Yeast, the Accelerator, 46 Shelby Thames Dr., Hattiesburg, Miss., 39402; Northern Brewer, 1306 S 108^{th} St., Milwaukee, Wis. 53214; Home Brew Supply, 1800 EDC Parkway, Comanche Tex. 76442, The Coca Cola Company, 1 Coca Cola, Plaza NW, Atlanta, Ga. 30313; Grafix Plastics, 5800 Pennsylvania Avenue, Maple Heights Ohio 44137; Barnstead Thermolyne Corporation, 14000 Unity Street, NW, Ramsey, Minn. 55303; etc. For example, various nucleotide sequences, including those that encode amino acid sequences, may be obtained at a public database, such as the Entrez Nucleotides database, which includes sequences from other databases including GenBank (e.g., CoreNucleotide), RefSeq, and protein data bank PDB. Another example of a public databank for nucleotide and amino acid sequences includes the Kyoto Encyclopedia of Genes and Genomes (“KEGG”) (Kanehisa, M. and Goto, S. Nucleic Acids Res. 28:27-30, 2000; Kanehisa, M. et al. Nucleic Acids Res. 34:D354-357, 2006; Kanehisa, M. et al. Nucleic Acids Res. 36:D480-D484, 2008). In another example, various amino acid sequences may be obtained at a public database, such as the Entrez databank, which includes sequences from other databases including SwissProt, protein information resource (PIR), Protein Research Foundation (PRF), PDB, Gene, GenBank, and RefSeq. Numerous nucleic acid sequences and/or encoded amino acid sequences can be obtained from such sources. In a further example, a biological material comprising, or are capable of comprising such a biomolecule (e.g., a living cell, a virus), may be obtained from a depository such as the American Type Culture Collection (“ATCC”), P.O. Box 1549 Manassas, Va. 20108, U.S.A. In an additional example, a biomolecule, a chemical reagent (e.g., a polymer), a biological material, and/or an equipment may be obtained from a commercial vendor such as Amersham Biosciences®, 800 Centennial Avenue, P.O. Box 1327, Piscataway, N.J. 08855-1327 U.S.A.; BD Biosciences®, including Clontech®, Discovery Labware®, Immunocytometry Systems® and Pharmingen®, 1020 East Meadow Circle, Palo Alto, Calif. 94303-4230 U.S.A.; Invitrogen™, 1600 Faraday Avenue, PO Box 6482, Carlsbad, Calif. 92008 U.S.A.; New England Biolabs®, 32 Tozer Road, Beverly, Mass. 01915-5599 U.S.A.; Merck®, One Merck Drive, P.O. Box 100, Whitehouse Station, N.J. 08889-0100 U.S.A.; Millipore Sigma, 400 Summit Drive, Burlington, Mass. 01803; EnvironMolds, LLC, 18 Bank St #1, Summit, N.J. 07901; Akshar Chem India Ltd., Indrad, Gujarat 382715, India; Anthony's Goods, Los Angeles, Calif.; Best Food Ingredients, Brownfield, Tex.; Elmer's Products, Inc., 6655 Peachtree Dunwoody Rd., Atlanta Ga., 30328; Best Food Ingredients LLC, 528 P.O. Box, 807 North 5^{th} Street, Brownfield, Tex. 79316; Anthony's Almonds, 820 Thompson, Unit 32, Glendale, Calif. 91201; Litever, 3F Qinghu Science and Technology Park, Qingxiang Road, Longhua, Bao'an District, Shenzhen, Guangdong, China; Novagene®, 441 Charmany Dr., Madison, Wis. 53719-1234 U.S.A.; Promega®, 2800 Woods Hollow Road, Madison Wis. 53711 U.S.A.; Pfizer®, including Pharmacia®, 235 East 42nd Street, New York, N.Y. 10017 U.S.A.; Quiagen®, 28159 Avenue Stanford, Valencia, Calif. 91355 U.S.A.; Sigma-Aldrich®, including Sigma, Aldrich, Fluka, Supelco and Sigma-Aldrich Fine Chemicals, PO Box 14508, Saint Louis, Mo. 63178 U.S.A.; Wako Pure Chemical Industries, Ltd, 1-2 Doshomachi 3-Chome, Chuo-ku, Osaka 540-8605, Japan; TCI America, 9211 N. Harborgate Street, Portland, Oreg. 97203, U.S.A.; Reactive Surfaces, Ltd, 300 West Avenue Ste #1316, Austin, Tex. 78701; Stratagene®, 11011 N. Torrey Pines Road, La Jolla, Calif. 92037 U.S.A., etc. In a further example, a biomolecule, a chemical reagent (e.g., a polymer), a biological material, and/or an equipment may be obtained from commercial vendors such as Amersham Biosciences®, 800 Centennial Avenue, P.O. Box 1327, Piscataway, N.J. 08855-1327 U.S.A.; Allen Bradley, 1201 South Second Street, Milwaukee, Wis. 53204-2496, U.S.A.; Corning Life Sciences, 836 North Street, Building 300 Suite 3401, Tewksbury, Mass. 01876; Bemis Company, Inc. Bemis Innovation Center, 2301 Industrial Dr., WI, 54956; JRM Chemical Inc., 15663 NEO Parkway, Cleveland, Ohio 44128; Exo-Novelty Corp, Corporation Michigan, 5100 Kings Gate Way Bloomfield Hills, Mich. 48302; Carolina Biological Supply Company, P.O. Box 6010, 2700 York Road, Burlington, N.C. 27216; Cleartec Packaging, 409 Parkway Drive, Park Hills, Mo. 63601; Thermo LabSystems Inc., 100 Cummings Center, Suite 407J, Beverly Mass. 01915; BD Biosciences®, including Clontech®, Discovery Labware®, Immunocytometry Systems® and Pharmingen®, 1020 East Meadow Circle, Palo Alto, Calif. 94303-4230 U.S.A.; Baker, Mallinckrodt Baker, Inc., 222 Red School Lane, Phillipsburg N.J. 08865, U.S.A.; J. T. Baker, Avantor, 100 Matsonford Road, Radnor, Pa. 19087; Bioexpression and Fermentation Facility, Life Sciences Building, 1057 Green Street, University of Georgia, Athens, Ga. 30602, U.S.A.; Bioxpress Scientific, PO Box 4140, Mulgrave Victoria 3170; Boehringer Ingelheim GmbH, Corporate Headquarters, Binger Str. 173, 55216 Ingelheim, Germany Chem Service, Inc, PO Box 599, West Chester, Pa. 19381-0599, U.S.A.; Chemko, a. s. Strážske, Priemyselná 720, 072 22 Strážske, Slovikia, Hungary; Difco, Voigt Global Distribution Inc., P.O. Box 1130, Lawrence, Kans. 66044-8130, U.S.A.; Fisher Scientific, 2000 Park Lane Drive, Pittsburgh, Pa. 15275, U.S.A.; Invitrogen™, 1600 Faraday Avenue, PO Box 6482, Carlsbad, Calif. 92008 U.S.A.; Ferro Pfanstiehl Laboratories, Inc., 1219 Glen Rock Avenue, Waukegan, Ill. 60085-0439, U.S.A.; New England Biolabs®, 32 Tozer Road, Beverly, Mass. 01915-5599 U.S.A.; Merck®, One Merck Drive, P.O. Box 100, Whitehouse Station, N.J. 08889-0100 U.S.A.; Novozymes North America Inc., PO BOX 576, 77 Perry Chapel Church Road, Franklinton N.C. 27525 United States; Millipore Corporate Headquarters, 290 Concord Rd., Billerica, Mass. 01821, U.S.A.; Nalgene® Labware, Nalge Nunc International, International Department, 75 Panorama Creek Drive, Rochester, N.Y. 14625. U.S.A.; New Brunswick Scientific Co., Inc., 44 Talmadge Road, Edison, N.J. 08817 U.S.A.; Novagene®, 441 Charmany Dr., Madison, Wis. 53719-1234 U.S.A.; NCSRT, Inc., 1000 Goodworth Drive, Apex, N.C. 27539, U.S.A.; Promega®, 2800 Woods Hollow Road, Madison Wis. 53711 U.S.A.; Pfizer®, including Pharmacia®, 235 East 42nd Street, New York, N.Y. 10017 U.S.A.; Quiagen®, 28159 Avenue Stanford, Valencia, Calif. 91355 U.S.A.; SciLog, Inc., 8845 South Greenview Drive, Suite 4, Middleton, Wis. 53562, U.S.A.; Sigma-Aldrich®, including Sigma, Aldrich, Fluka, Supelco, and Sigma-Aldrich Fine Chemicals, PO Box 14508, Saint Louis; USB Corporation, 26111 Miles Road, Cleveland, Ohio 44128, U.S.A.; Sashco®, 10300 East 107^{th} Place, Brighton, Colo. 80601; Safco Products Company, 9300 West Research Center Road, New Hope, Minn. 55428; Cake Supplies On Sale, 7706 Backlick Road, Unit I Springfield, Va. 22150; Liquid Latex Fashions, 262 Valley Rd #1, Warrington, Pa. 18976; Rust-Oleum Corporation, 11 E Hawthorn Pkwy, Vernon Hills, Ill. 60061; Biotium, 46117 Landing Pkwy, Fremont, Calif. 94538; MP Biomedicals, LLC, 3 Hutton Center Drive, Suite 100, Santa Ana, Calif. 92707; Shenzhen Vansky Technology Co. Ltd., Shenzhen City, China; Sherwin Williams Company, 101 Prospect Ave., Cleveland, Ohio, U.S.A.; Lightnin, 135 Mt. Read Blvd., Rochester, N.Y. 14611 U.S.A.; Amano Enzyme, U.S.A. Co., Ltd. 2150 Point Boulevard Suite 100 Elgin, Ill. 60123 U.S.A.; Mallinckrodt Pharmaceuticals, 675 McDonnell Blvd., St. Louis, Mo. 63042; Novozymes North America Inc., 77 Perry Chapel Church Road, Franklinton, N.C. 27525, U.S.A.; and WB Moore, Inc., 1049 Bushkill Drive, Easton, Pa. 18042.

### SPECIFIC EXAMPLES

The general effectiveness of various embodiments is demonstrated in the following Examples. Some methods for preparing compositions are illustrated. Starting materials are made according to procedures known in the art or as illustrated herein. The following Examples are provided so that the embodiments might be more fully understood. These Examples are illustrative only and should not be construed as limiting in any way, as other material formulations such as a polymeric material and/or a coating comprising different gas (e.g., carbon dioxide) capture cells and apparatus or devices comprising such a gas capture polymeric material and/or coating may be prepared.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the embodiments of the present invention. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

### Example 1: Preparation of Carbon Dioxide Capture Organisms

For algae growth media, Blue-Green Medium 11 (“BG-11”) liquid and solid media were prepared as described (Bustos, S. A. and Golden, S. S. Mol Gen Genet. 232:221-230, 1992) using the following technique. A BG-11 trace metal stock solution was prepared by adding the reagents shown in Table 1, in the order shown, into 900 mL of stirred distilled H₂O (“dH₂O”).

TABLE 1
BG-11 Trace Metal Stock Solution

| Reagent | Source |
|---|---|
| 2.86 g H₃BO₃ | Carolina Biological Supply Company, P.O. |
| | Box 6010, 2700 York Road, Burlington, NC |
| | 27216 U.S.A. (“Carolina Biological Supply |
| | Company”); item no. 84-8450 |
| 1.81 g MnCl₂ | Sigma-Aldrich ® (also known as Millipore Sigma), |
| 4H₂O | 400 Summit Drive, Burlington, MA 01803 U.S.A., |
| | also includes Sigma brands; referred to herein |
| | as “Sigma,” “Sigma-Aldrich,' and |
| | “Millipore Sigma”); Sigma |
| | product no. M3634 |
| 0.22 g ZnSO₄ | Carolina Biological Supply Company item |
| 7H₂O | no. 89-9464 |
| 0.39 g Na₂MoO₄ | Sigma product no. M1651 |
| 2H₂O | |
| 0.051 g CuSO₄ | Sigma-Aldrich product no. 451657 |
| 49.4 mg Co(NO₃) | Carolina Biological Supply Company item |
| 6H₂O | no. 85-4990 |

Additional dH₂O was added as needed to produce a final volume of 1 liter, and the BG-11 trace metal stock solution was stored at room temperature.

To produce BG-11 liquid medium, stock solutions of the following reagents shown in Table 2 below were each prepared by mixing the listed reagent amount into 200 mL dH₂O.

TABLE 2
BG-11 Liquid Medium Stock Solutions

| Reagent | Source |
|---|---|
| 30 g NaNO₃ | Sigma-Aldrich product no. S5506 |
| 0.8 g K₂HPO₄ | Sigma product no. P 3786 |
| 1.5 g MgSO₄ 7H₂O | Sigma product no. M1880 |
| 0.72 g CaCl₂ 2H₂O | Carolina Biological Supply Company |
| | item no. 85-1800 |
| 0.12 g citric acid•H₂O | Carolina Biological Supply Company |
| | item no. 854770 |
| 0.12 g ferric ammonium citrate | Sigma Aldrich product no. F5879 |
| 0.02 g Na₂EDTA•2H₂O (EDTA | Carolina Biological Supply Company |
| is ethylenediaminetetraacetic | item no. 86-1778 |
| acid) | |
| 0.4 g Na₂CO₃ | Carolina Biological Supply Company |
| | item no. 88-8770 |
| 49.6 g sodium thiosulfate | Sigma Aldrich product no. 72049 |
| pentahydrate (sterilized only for | |
| agar medium). | |

Each stock solution was then added in the order and volumes shown at Table 3 below into 900 mL of stirred dH₂O.

TABLE 3
Stock Solutions Added to dH₂O to
Produce BG-11 Media

```
Reagent Stock Solution
10 mL NaNO₃ stock solution
10 mL K₂HPO₄ stock solution
10 mL MgSO₄ 7H₂O stock solution
10 mL CaCl₂ 2H₂O stock solution
10 mL citric acid•H₂O stock solution
10 mL ferric ammonium citrate stock solution
10 mL Na₂EDTA•2H₂O stock solution
10 mL Na₂CO₃ stock solution
  1 mL BG-11 trace metals stock solution
  1 mL sodium thiosulfate pentahydrate stock
solution (sterilized only for agar medium)
```

Additional dH₂O was added as needed to the 900 mL plus stock solutions to produce a final volume of 1 liter, and the BG-11 medium was covered, autoclaved for 45 minutes, and then stored at room temperature.

To produce BG-11 agar medium, stock solutions of the reagents shown in Table 3 above were added to 400 mL of stirred dH₂O in the same order and volume as for preparation of the BG-11 liquid medium, except the sodium thiosulfate pentahydrate stock solution was not added. Additional dH₂O was added as needed to produce a final volume of 500 mL. To a separate container of 500 mL dH₂O, 15 grams of agar was added, and both containers covered, autoclaved for 45 minutes, and placed in a water bath to cool to about 45-55° C. Then 1 mL of the sodium thiosulfate pentahydrate stock solution per Table 3 above was mixed thoroughly into the agar solution; and the two solutions mixed thoroughly to create BG-11 agar medium for use (e.g., pouring into Petri dishes to prepare BG-11 agar plates). The BG-11 agar medium was stored at refrigerated temperatures.

The photosynthetic organism evaluated was the cyanobacteria (algae) Synechococcus leopoliensis [strain UTEX 2434; source UTEX Culture Collection of Algae, 205 W 24th St, Biological Labs 218, The University of Texas at Austin, Tex. 78712 U.S.A. (“UTEX”)]. The cyanobacteria was cultured using one of two different techniques 1) in flasks, in which the BG-11 medium volume was ⅕ the total flask capacity (e.g., 10 mL medium per 50 mL flask; 50 mL medium per 250 mL flask), which were incubated with gentle shaking at 100 revolutions per minute (“rpm”), or 2) in 1 L bottles with 1 L BG-11 medium, which were incubated with filtered house air bubbling through them.

In either culture technique, a culture was grown until the culture had reached an optical density (“OD”) measured at 540 nm (“OD₅₄₀”) of about 1. All cultures were incubated at ambient temperature (about 20-23° C.) under grow lights [about 70 μmol photons m⁻² s⁻¹ photosynthetic active radiation (“PAR”) for flasks and 15 PAR for the 1 L bottles during growth from one or more 9.6 Watt extendable 16 inch emitting diode (“LED”) grow light strips of a 4-piece set for greenhouse, plant grow shelf, (source Litever, 3F Qinghu Science and Technology Park, Qingxiang Road, Longhua, Bao'an District, Shenzhen, Guangdong, China (“Litever”); as used herein (“grow lights”)].

To prepare wet cell pellets of cyanobacteria, the cyanobacterial cultures that had reached OD₅₄₀ of about 1 were centrifuged at 3000× gravity (“g”) for 15 minutes. The supernatant was poured off, and the remaining cell pellet was resuspended in the remaining liquid BG-11 medium for a total volume of approximately 1/100 of the original volume to make the wet cell pellet.

### Example 2: Creating a Carbon Dioxide Capture Device

Solid polymer powder or granule of the polymers shown at Table 4 below were mixed with deionized water until the water was absorbed or a solution was achieved.

TABLE 4
Polymers Used in Algae-Containing Coating

| Polymeric Material | Source |
|---|---|
| Alginate (preformulated | LifeMold Alginate, EnvironMolds, LLC, |
| to gel upon | 18 Bank St #1, Summit, NJ 07901 U.S.A. |
| addition of water) | (“Environmolds”) |
| Hydroxy ethyl cellulose | Akshar Chem India Ltd., Indrad, Gujarat |
| powder (“HEC”) | 382715, India (“Akshar Chem India Ltd.”) |
| Xanthan gum powder | Anthony's Almonds, 820 Thompson, Unit |
| (food grade) | 32, Glendale,, CA 91201 U.S.A. |
| | (“Anthony's Almonds”) |
| Guar gum powder | Best Food Ingredients LLC, 528 P.O. Box, |
| (food grade) | 807 North 5th Street, Brownfield, TX |
| | 79316 U.S.A. (“Best Food Ingredients |
| | LLC”) |
| Polyvinyl acetate solution | Elmer's Products, Inc., 6655 Peachtree |
| (“PVAc”; in | Dunwoody Rd., Atlanta GA, 30328 |
| the form of Elmer's clear | U.S.A. |
| glue used as received) | (“Elmer's Products, Inc.”) |

The alginate polymer was a hydrogel polymer, which is a crosslinked polymer that absorbs water, but does not dissolve. The other polymers were water soluble hydrophilic polymers. An aliquot of 30 g of each hydrated polymer or 30 g of selected mixtures of hydrated polymers, as shown in Table 5 below, was mixed by hand with 0.3 g of the Synechococcus leopoliensis (UTEX 2434) wet cell pellet from Example 1 until a consistent color was achieved throughout each polymer/cyanobacteria mixture. Table 5 below shows the percent weight (“wt %”) of each polymer as part of each polymer/cyanobacteria mixture's total weight.

TABLE 5
Polymers Mixed With Algae Cell Pellet

Guar gum (2 wt %)
Hydroxy ethyl cellulose (2 wt %)
Xanthan gum (2 wt %)
1:1 guar gum:xanthan gum (2 wt % total)
Polyvinyl acetate solution (undiluted)
Hydroxyl ethyl cellulose (2 wt %) + 10 wt % polyvinyl acetate solution
1:1 guar:xanthan (2 wt %) + 10 wt % polyvinyl acetate solution
Alginate (1:7 alginate:deionized water)

Each polymer/cyanobacteria mixture was then added to a separate polyethylene terephthalate (“PET”) bottle, and the bottle rotated to coat the as much of the inner surface walls as possible. For one configuration of carbon dioxide capture device, round PET bottles previously used as beverage containers [e.g., 500 mL PET beverage bottles that contained a Coke® product (The Coca Cola Company, 1 Coca Cola, Plaza NW, Atlanta, Ga. 30313 U.S.A.)] were used. For another configuration of carbon dioxide capture device, square sided PET bottles previously used as beverage container (e.g., 500 mL PET beverage bottles that contained a juice) were used. In preparing these carbon dioxide capture devices, the alginate/cyanobacteria mixture began as a flowing liquid, and the bottle was continuously rotated about the bottle's long axis until the mixture gelled into a non-flowing state uniformly on the interior surface of the side walls of the beverage bottle.

### Example 3: Monitoring CO₂ Sequestration by Carbon Dioxide Capture Devices

Carbon dioxide captured by devices prepared in accordance with Example 2 was measured using a Vernier® Go Direct CO₂ sensor [Vernier Software & Technology, 13979 SW Millikan Way, Beaverton, Oreg. 97005 U.S.A. (“Vernier Software & Technology”)]. For monitoring carbon dioxide capture by a PET square-sided juice bottle prepared as a carbon dioxide capture device in the manner described above in Example 2, the square-sided juice bottle. For monitoring coated beverage bottles each prepared as a carbon dioxide capture device in the manner described above in Example 2, were placed in a plastic sleeve (3 inch ultra-thin wall×48 inch tube, polyethylene terephthalate glycol, product no. PRT00107) with end caps (product no. PCC3.000) on each end to seal the interior space of the plastic sleeve [sleeve and caps both available from Cleartec Packaging, 409 Parkway Drive, Park Hills, Mo. 63601 U.S.A. (“Cleartec Packaging”)]. One end cap was modified to allow the CO₂ sensor to be placed into the interior space of the plastic sleeve.

All CO₂ capture monitoring described in this Example was conducted while the carbon dioxide capture devices were illuminated at 250 PAR by the grow lights, unless otherwise indicated. The CO₂ concentration in ppm was measured over a defined period of time using Vernier® Graphical Analysis software (Vernier Software & Technology). The ppm CO₂ concentration was plotted against the time data to obtain a linear relationship and the slope was measured as the capture of CO₂ in ppm per unit time. This value was converted to a rate of mmol CO₂ hr⁻¹ m⁻², with a positive rate occurring when carbon dioxide was captured. The rate in mmol CO₂ hr⁻¹ m⁻² was based on the total volume of the device (e.g., coated container, coated beverage bottle) in which measurement occurred as well as the total area in m² that the carbon dioxide capture coating covered. The beverage bottle of each carbon dioxide capture device was calculated to have an approximate 0.5 L volume and 0.04 m² surface area inside; a PET square-sided juice bottle was calculated to have an approximate 0.5 L volume and a 0.03 m² inside surface area.

The CO₂ capture rates for the coated square sided juice bottles are show in Tables 6 to 13 below.

TABLE 6
CO₂ Capture over 1 hour for the Xanthan
Gum/Synechococcus leopoliensis (UTEX 2434) Coated
Device on Days 0, 5 and 9.

| | CO₂, Gas (ppm) | | |
|---|---|---|---|
| Time (min) | Day 0 | Day 5 | Day 9 |
| 0 | 612 | 807 | 593 |
| 5 | 510 | 548 | 508 |
| 10 | 496 | 490 | 445 |
| 15 | 456 | 421 | 401 |
| 20 | 432 | 378 | 363 |
| 25 | 397 | 338 | 326 |
| 30 | 374 | 288 | 286 |
| 35 | 347 | 245 | 246 |
| 40 | 323 | 206 | 204 |
| 45 | 300 | 165 | 165 |
| 50 | 278 | 134 | 124 |
| 55 | 257 | 105 | 88 |
| 60 | 234 | N/A* | 55 |

*Value not included as carbon dioxide activity reached 0 then increased due to respiration.

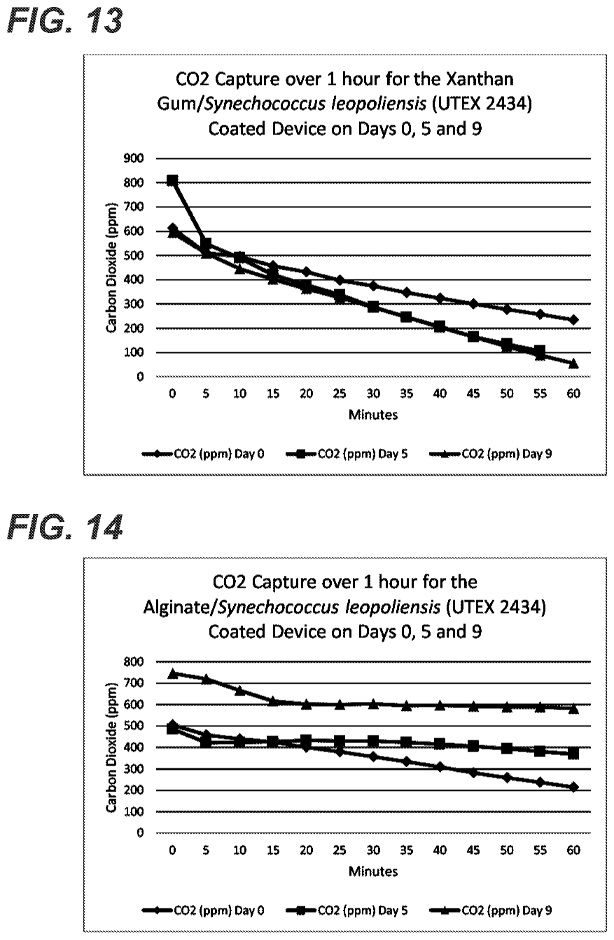

The CO₂ capture rate on day 0 was determined to be 0.25 mmol CO₂/hr/m²; while the CO₂ capture rate on day 5 was 0.48 mmol CO₂/hr/m²; and the CO₂ capture rate on day 9 was 0.38 mmol CO₂/hr/m² (FIG. 13). These rates demonstrate CO₂ capture by the CO₂ sequestration device. The equation for the slope fitted line of the CO₂ capture over 1 hour for the xanthan gum/Synechococcus leopoliensis (UTEX 2434) coated device on day 0 was determined to be −5.6462x+555.26; while on day 5 the equation was −10.656x+636.74; and on day 9 the equation was −8.4802x+547.05.

Given a 0.04 m² surface area inside of the carbon dioxide capturing device having the xanthan gum/Synechococcus leopoliensis (UTEX 2434) carbon dioxide capturing coating, then 480 of such carbon dioxide capturing devices (e.g., 5 carbon dioxide capturing devices per sleeve described in Example 6, with 96 sleeves in about a 1 m³ space having a 1-square meter plan view footprint) would provide a coated substrate surface area of about 19.2 m² (i.e., a densification factor of about 19×). A coated substrate surface area of 19.2 m², where the coating substrate has a thickness of about 4 mm (i.e., 0.004 m), provides a total coated substrate volume of 0.08 m³ or a volumetric utilization in a 1 m³ space of about 8%. At 1 year of the Day-9 carbon dioxide capture rate about 0.38 mmol CO₂/hr/m² (i.e., about 0.15 kg/yr/m²), the total amount of carbon dioxide captured by a carbon dioxide farm comprising 480 such carbon dioxide capturing devices in an approximate 1 m³ area would be about 2.8 kilograms (or 0.003 metric tons per year).

TABLE 7
CO₂ Capture over 1 hour for the
Alginate/Synechococcus leopoliensis (UTEX 2434)
Coated Device on Days 0, 5 and 9.

| | CO₂ Gas (ppm) | | |
|---|---|---|---|
| Time (min) | Day 0 | Day 5 | Day 9 |
| 0 | 506 | 486 | 745 |
| 5 | 458 | 422 | 719 |
| 10 | 440 | 423 | 665 |
| 15 | 425 | 427 | 617 |
| 20 | 401 | 434 | 602 |
| 25 | 380 | 431 | 600 |
| 30 | 357 | 431 | 603 |
| 35 | 334 | 423 | 595 |
| 40 | 309 | 415 | 596 |
| 45 | 282 | 405 | 592 |
| 50 | 258 | 394 | 588 |
| 55 | 237 | 382 | 588 |
| 60 | 214 | 369 | 582 |

The CO₂ capture rate on day 0 was determined to be 0.21 mmol CO₂/hr/m²; while the CO₂ capture rate on day 5 was 0.06 mmol CO₂/hr/m²; and the CO₂ capture rate on day 9 was 0.10 mmol CO₂/hr/m² (FIG. 14). These rates demonstrate CO₂ capture by the CO₂ sequestration device. The equation for the slope fitted line of the CO₂ capture over 1 hour for the alginate/Synechococcus leopoliensis (UTEX 2434) coated device on day 0 was determined to be −4.6612x+493.81; while on day 5 the equation was −1.244x+456.01; and on day 9 the equation was −2.2352x+689.47. At 1 year at the day 9 carbon dioxide capture rate, the total amount of carbon dioxide captured by a carbon dioxide capture farm comprising 480 such carbon dioxide capturing devices in an approximate 1 m³ area would be about 0.74 kilograms.

TABLE 8
CO₂ Capture of the Guar
Gum/Synechococcus leopoliensis (UTEX 2434)
Coated Device on Days 0 and 5.

| | CO₂, Gas (ppm) | |
|---|---|---|
| Time (min) | Day 0 | Day 5 |
| 0 | 769 | 1093 |
| 5 | 856 | 1444 |
| 10 | 916 | 1572 |
| 15 | 974 | 1668 |
| 20 | 1041 | 1772 |
| 25 | 1108 | 1856 |
| 30 | 1174 | 1934 |
| 35 | 1232 | 2012 |
| 40 | 1288 | 2084 |
| 45 | 1342 | 2179 |
| 50 | 1393 | 2271 |
| 55 | 1450 | 2365 |
| 60 | 1498 | 2449 |

The CO₂ capture rate on day 0 was determined to be −0.540 mmol CO₂/hr/m², and on day 5 determined to be −0.878 mmol CO₂/hr/m². The equation for the slope fitted line of the CO₂ capture of the guar gum/Synechococcus leopoliensis (UTEX 2434) coated device on day 0 was determined to be 12.06x+795.15; while the equation on day 5 was 19.623x+1311.4.

TABLE 9
CO₂ Capture of the Hydroxyethyl
Cellulose/Synechococcus leopoliensis (UTEX
2434) Coat Device on Day 0.

| Time (min) | CO₂, Gas (ppm) |
|---|---|
| 0 | 714 |
| 5 | 811 |
| 10 | 835 |
| 15 | 851 |
| 20 | 864 |
| 25 | 884 |
| 30 | 898 |
| 35 | 916 |
| 40 | 940 |
| 45 | 960 |
| 50 | 983 |
| 55 | 1012 |
| 60 | 1038 |

The CO₂ capture rate was determined to be −0.199 mmol CO₂/hr/m². The equation for the slope fitted line of the CO₂ capture of the hydroxyethyl cellulose/Synechococcus leopoliensis (UTEX 2434) coated device was determined on day 0 to be 4.4498x+766.97.

TABLE 10
CO₂ Capture of the 2 wt %
Hydroxyethyl Cellulose with 10 wt %
Polyvinyl Acetate/Synechococcus
leopoliensis (UTEX 2434) Coated Device on Day 0.

| Time (min) | CO₂, Gas (ppm) |
|---|---|
| 0 | 527 |
| 5 | 474 |
| 10 | 469 |
| 15 | 466 |
| 20 | 466 |
| 25 | 464 |
| 30 | 462 |
| 35 | 463 |
| 40 | 463 |
| 45 | 460 |
| 50 | 457 |
| 55 | 458 |
| 60 | 459 |

The CO₂ capture rate was determined to be 0.027 mmol CO₂/hr/m². The equation for the slope fitted line of the CO₂ capture of the 2% hydroxyethyl cellulose with 10% polyvinyl acetate/Synechococcus leopoliensis (UTEX 2434) coated device was determined on day 0 to be −0.6099x+486.58.

TABLE 11
CO₂ Capture of the Polyvinyl Acetate/Synechococcus leopoliensis
(UTEX 2434) Coated Device on Day 0

| Time (min) | CO₂, Gas (ppm) |
|---|---|
| 0 | 556 |
| 5 | 473 |
| 10 | 473 |
| 15 | 476 |
| 20 | 477 |
| 25 | 479 |
| 30 | 482 |
| 35 | 481 |
| 40 | 483 |
| 45 | 482 |
| 50 | 480 |
| 55 | 482 |
| 60 | 483 |

The CO₂ capture rate was determined to be 0.017 mmol CO₂/hr/m². The equation for the slope fitted line of the CO₂ capture of the polyvinyl acetate/Synechococcus leopoliensis (UTEX 2434) coated device on day 0 was determined to be −0.3692x+496.26.

TABLE 12
CO₂ Capture of the 1:1 Guar:Xanthan Gum/Synechococcus leopoliensis
(UTEX 2434) Coated Device on Day 0.

| Time (min) | CO₂, Gas (ppm) |
|---|---|
| 0 | 447 |
| 5 | 569 |
| 10 | 599 |
| 15 | 626 |
| 20 | 653 |
| 25 | 681 |
| 30 | 703 |
| 35 | 731 |
| 40 | 759 |
| 45 | 785 |
| 50 | 818 |
| 55 | 852 |
| 60 | 886 |

The CO₂ capture rate was determined to be −0.295 mmol CO₂/hr/m². The equation for the slope fitted line of the CO₂ capture of the 1:1 guar:xanthan gum/Synechococcus leopoliensis (UTEX 2434) coated device on day 0 was determined to be −6.222x+514.06.

TABLE 13
CO₂ Capture of the 1:1 Guar:Xanthan Gum
(2 wt %) with 10 wt % polyvinyl acetate/Synechococcus
leopoliensis (UTEX 2434) Coated Device on Day 0.

| Time (min) | CO₂, Gas (ppm) |
|---|---|
| 0 | 503 |
| 5 | 511 |
| 10 | 509 |
| 15 | 509 |
| 20 | 509 |
| 25 | 509 |
| 30 | 511 |
| 35 | 505 |
| 40 | 506 |
| 45 | 505 |
| 50 | 506 |
| 55 | 504 |
| 60 | 505 |

The CO₂ capture rate was determined to be 0.003 mmol CO₂/hr/m². The equation for the slope fitted line of the CO₂ capture study of the 1:1 guar:xanthan gum with 10 wt % polyvinyl acetate/Synechococcus leopoliensis (UTEX 2434) coated device on day 0 was determined to be −0.0615x+508.92.

### Example 4: Monitoring Cell Viability in Carbon Dioxide Capture Coatings

CellTiter 96® AQueous One Solution [“MTS”; source Promega®, 2800 Woods Hollow Road, Madison Wis. 53711 U.S.A. (“Promega”)], was used to assay the viability of the cells in the 8 different polymer/Synechococcus leopoliensis (UTEX 2434) formulations of Example 2 by coating the bottoms of the wells in a 96-well microplate with 0.05 g of coating for both control (no algae added) and polymer/algae coatings. The plates were covered and left at a window facing the outside environment (e.g., indirect sunlight illumination) and viability assays conducted over time. On the first day of the viability assay, 100 μL of BG-11 liquid growth medium was added followed by 20 μL of the MTS solution to one well of each of the coating types. The coatings were taken indoors and incubated under normal indoor lighting at 25° C. with rocking in this solution for 4 hours. After the incubation period, the contents were transferred to microcentrifuge tubes, centrifuged at 13,000 rpm for 10 minutes, and 60-80 μL of supernatant was transferred to new wells in a 96-well microplate. The absorbance at 492 nm was measured using a Multiskan Ascent microplate reader [Thermo LabSystems Inc., 100 Cummings Center, Suite 407J, Beverly Mass. 01915 U.S.A. (“Thermo Lab Systems, Inc.”)].

Because free cells (cells not mixed in a polymer system or growth medium) would not be viable over the time period monitored, the assay results were calculated by subtracting the polymer coating control with no algae measurement from the polymer/algae coating measurement at each timepoint. The percent increase or decrease in viability was calculated between each measurement time point. Table 14 below shows the absorbance at 492 nm for each of the control and polymer/algae coatings at different time points.

TABLE 14
Absorbance at 492 nm for Polymer/Algae and Control Coatings after
5 Day and 8 Day Incubation

| | Day 5 | Day 8 |
|---|---|---|
| Coating Controls (No Algae) | | |
| Hydroxy ethyl cellulose (2 wt %) | 0.1397 | 0.192 |
| Guar gum (2 wt %) | 0.2923 | 0.6243 |
| Xanthan gum (2 wt %) | 0.3276 | 0.4454 |
| 1:1 guar gum:xanthan gum (2 wt % total) | 0.2158 | 0.2858 |
| Alginate (1:7 alginate:deionized water) | 0.1722 | 0.2663 |
| Polyvinyl acetate solution (undiluted) | 0.121 | 0.1854 |
| Hydroxyl ethyl cellulose (2 wt %) + 10 wt % | 0.2699 | 0.2326 |
| polyvinyl acetate solution | | |
| 1:1 guar:xanthan (2 wt %) + 10 wt % | 0.155 | 0.2194 |
| polyvinyl acetate solution | | |
| Polymer/Algae Coatings | | |
| Hydroxy ethyl cellulose (2 wt %) | 1.4555 | 0.9241 |
| Guar gum (2 wt %) | 1.5922 | 2.4677 |
| Xanthan gum (2 wt %) | 1.4318 | 2.0752 |
| 1:1 guar gum:xanthan gum (2 wt % total) | 2.1477 | 3.3792 |
| Alginate (1:7 alginate:deionized water) | 0.7492 | 1.5724 |
| Polyvinyl acetate solution (undiluted) | 0.0995 | 0.1819 |
| Hydroxyl ethyl cellulose (2 wt %) + 10 wt % | 0.2475 | 0.1714 |
| polyvinyl acetate solution | | |
| 1:1 guar:xanthan (2 wt %) + 10 wt % | 0.3891 | 0.3474 |
| polyvinyl acetate solution | | |

Visible darkening of the assay solution in the wells (e.g., to a red color) was indicative of viable algae cells. Two days after plating, all the samples containing polyvinyl acetate showed no viability. Six days after plating, xanthan gum showed an increase in viable algae cells. Mold was visibly growing in the hydroxy ethyl cellulose/algae coating wells which interfered in interpreting those viability results. The baseline corrected values and percent increase or decrease in viability is shown in Table 15 below.

TABLE 15
Polymer/Algae Coatings 5 Day and 8 Day Incubation Viability

| Polymer/Algae Coatings | Day 5 | Day 8 | % difference |
|---|---|---|---|
| Hydroxy ethyl cellulose (2 wt %) | 1.3158 | 0.7321 | −57 |
| Guar gum (2 wt %) | 1.2999 | 1.8434 | 35 |
| Xanthan gum (2 wt %) | 1.1042 | 1.6298 | 38 |
| 1:1 guar gum: xanthan gum | 1.9319 | 3.0934 | 46 |
| (2 wt % total) | | | |
| Alginate (1:7 alginate:deionized | 0.577 | 1.3061 | 77 |
| water) | | | |
| Polyvinyl acetate solution | −0.0215 | −0.0035 | not viable |
| (undiluted) | | | |
| Hydroxyl ethyl cellulose (2 wt %) + | −0.0224 | −0.0612 | not viable |
| 10 wt % polyvinyl acetate solution | | | |
| 1:1 guar:xanthan (2 wt %) + 10 | 0.2341 | 0.128 | not viable |
| wt % polyvinyl acetate solution | | | |

These results demonstrate that this viability assay can screen materials, such as polymeric materials or other materials, for compatibility with individual cell (e.g., algae) types. For example, a strain of Spirulina sp. [Raw Living Spirulina, 6885 57th St, Vero Beach, Fla. 32967 U.S.A. (“Raw Living Spirulina”)] was similarly assayed as the Synechococcus leopoliensis described herein and demonstrated good compatibility in polyvinyl acetate.

### Example 5: Monitoring Cell Viability and Coating Adhesion in Carbon Dioxide Capture Device

The carbon dioxide capture organism's viability in a coating or as coated in a carbon dioxide capture device as indicated in the viability assay of Example 4 was also seen visually by inspecting the carbon dioxide capture devices of Examples 2 and 3 (either uncapped or loosely capped to allow air exchange) after 2 and 8 days of standing upright (as described in Example 4) in the window facing the exterior environment. The polyvinyl acetate/algae containing bottles turned blue within 24 hours indicating algae cell death. The hydroxy ethyl cellulose/algae containing bottles were contaminated with mold throughout the coating. Coating adhesion on bottle surfaces' (e.g., vertical and inner top surfaces) was also determined by visual inspection, with the hydroxy ethyl cellulose/algae and guar gum/algae coatings having a viscosity reduction from the day they were made resulting in little to no coating remaining on the upright bottles' walls.

### Example 6: Method for Preparing Carbon Dioxide Capture Device Comprising Alga/Polymer Coated Bottles in a Transparent Support Sleeve

This 2-day procedure, described below, has been used to prepare 5 alga/polymer coated beverage (e.g., Coke®) bottles held in a clear plastic sleeve using cells prepared in accordance with Example 1 and polymers as described in Example 2. Positioning the materials in an assembly line enhanced the efficiency of coating the bottles and assembling the carbon dioxide capture device. Rubber gloves were worn during the procedure.

On the first day, 300 g of 2 wt % xanthan gum solution was prepared by adding 6 g of xanthan gum powder slowly with stirring to 294 mL of dH₂O. The xanthan gum/dH₂O mixture sat overnight for the polymer to hydrate by absorbing all the water to produce a hydrated 2 wt % xanthan gum solution.

On the second day, 12 g of the hydrated 2 wt % xanthan gum solution was weighed in each of 5 weigh boats. In each of 5 additional weigh boats 6 g of alginate powder was weighed. Into each of 5 clear plastic cups 42 mL of dH₂O (e.g., measured with a 50 mL graduated cylinder) was poured. At least 1.5 mL of a wet cell pellet of Synechococcus leopoliensis (UTEX 2434; source UTEX) was prepared. The wet cell pellet was pipetted into a 15 mL conical tube [Falcon™ tube, Corning Life Sciences, 836 North Street, Building 300 Suite 3401 U.S.A. (“Corning Life Sciences”)]. S. leopoliensis (300 or 0.3 g) from the conical tube was transferred using a calibrated pipette into each of the clear plastic cups containing 42 mL of dH₂O. The 5 beverage bottles were positioned on their sides with their caps removed.

The alginate converts from a liquid mixture to a gel within about 3 to 6 minutes after mixing with water, so the next steps were preferably completed before that time, which was fostered by different entities (e.g., people) mixing components and rotating beverage containers as described below.

6 g of alginate powder was added into each separate plastic cup of S. leopoliensis in 42 mL of dH₂O and quickly mixed with a stick (e.g., mixed with a wooden stick such as a popsicle stick) until the alginate/S. leopoliensis mixture only had a few clumps remaining (about 1-5 minutes of mixing). 12 g of the hydrated 2 wt % xanthan gum solution was added into each separate plastic cup of alginate/S. leopoliensis mixture and quickly mixed with the stick. As an alternative procedure, the 6 g of alginate powder was mixed with 42 mL of dH₂O per plastic cup, then 12 g of the hydrated 2 wt % xanthan gum solution added to each cup of alginate, followed by mixing in the 300 μL or 0.3 g of S. leopoliensis per cup. When an alginate/S. leopoliensis mixture (about 60.3 g) in a plastic cup had few clumps remaining, then the mixture was poured into a separate beverage bottle while rotating the bottle. The bottle was rotated continuously for several minutes until most or all of the inside surface was coated by the mixture and the mixture had solidified completely.

To determine if the different coating preparation procedures affect the carbon dioxide capture rates of the devices produced, a carbon dioxide capture device (1 bottle) was prepared by mixing the xanthan gum solution into the alginate/S. leopoliensis mixture (herein “Device A”) and a carbon dioxide capture device (1 bottle) was prepared by the alternative procedure of mixing the polymers then mixing the S. leopoliensis with the polymers last (herein “Device B”), as described above Each carbon dioxide capture device was measured for its carbon dioxide capture rate in accordance with Example 3 for 1 hour on day 0. Both carbon dioxide capture device had similar rates of carbon dioxide capture, with Device A having a rate of 0.49 mmol CO₂/hr/m² and Device B having a rate of 0.46 mmol CO₂/hr/m².

Each of the 5 coated beverage bottles were placed upright (cap on top) in a plastic sleeve (Cleartec Packaging product no. PRT00107) so that the sleeve now has 5 loosely stacked coated beverage bottles (carbon dioxide capture devices). If desired, a sleeve cap [e.g., a black foam circle or an end cap (product no. PCC3.000, Cleartec Packaging)] was placed into the top of the sleeve to keep particles from falling in. The sleeve(s) were optionally placed into a support structure to position upright [e.g., an architect's blueprint holder such as Model #3089 of Safco Products Company, 9300 West Research Center Road, New Hope, Minn. 55428 U.S.A. (“Safco Products”)]. When a sleeve containing carbon dioxide capture bottles was measured with a carbon dioxide sensor for CO₂ uptake (Vernier® Go Direct CO₂ sensor; Vernier Software & Technology), Parafilm™ [a polyolefin/wax blend flexible film; Bemis Company, Inc. Bemis Innovation Center, 2301 Industrial Dr., WI, 54956 U.S.A. (“Bemis Company, Inc.”)] or a sleeve cap modified with a hole to the fit the sensor was used to seal the caps from gas exchange with the outside air.

### Example 7: Alga/Polymer Carbon Dioxide Capture Coatings

Algal cells (Synechococcus leopoliensis i.e., UTEX 2434; Synechocystis sp. i.e., UTEX 2470; Gloeocapsa alpicola i.e., UTEX LB 1598; Agmenellum quadruplicatum i.e., UTEX 2268; all from UTEX) were grown to about an OD₅₄₀ of 1 and centrifuged at 3000×g for 15 minutes at 4° C. The supernatant was removed and the wet cell pellet resuspended so that the total volume was 1/100 of the original culture. Whenever the OD₅₄₀ was less than 1, the total volume of the wet cell pellet was adjusted proportionally so that all of the wet cell pellets have roughly the same concentration of cells. To produce cell suspensions, one hundred microliters of the wet cell pellet was mixed with either 100 of BG-11 liquid medium or 100 μL of acrylic latex containing biocide(s) [Sherwin-Williams Company, 101 Prospect Ave., Cleveland, Ohio, 44115 U.S.A. (“Sherwin-Williams”)], adjusted to pH 7 (note: this same acrylic latex was used in subsequent Examples described herein below). Algal control medium or acrylic latex samples were also prepared. The cell suspensions were pipetted onto 0.8 μm nylon filters (Millipore Sigma, 400 Summit Drive, Burlington, Mass. 01803 U.S.A.) about 10 cm² in area. The filters were allowed to dry about 5-10 minutes, then placed onto the surface of BG-11 agar. The agar was cut around the filter so that the filter was sitting on an agar “puck.” The filter and agar puck were transferred to a 250 mL clear plastic chamber, and the CO₂ concentration was measured using a Vernier Go Direct CO₂ sensor (Vernier Software & Technology). The sensor outputted the CO₂ concentration in ppm every 5 minutes for about an hour, which was plotted as ppm vs. time in minutes to get the rate of CO₂ capture over time from the slope of the line. This CO₂ capture rate was used to calculate the CO₂ capture rate as mmol CO₂/hr/m². The CO₂ capture of acrylic latex only samples and agar puck cell suspensions samples for various strains of cyanobacteria over different days are shown at Table 16 below.

TABLE 16
CO₂ Capture Rates of Alga/Acrylic Latex Coatings

| | | Cell Suspension | Cell Suspension |
|---|---|---|---|
| | | on Agar Puck | on Agar Puck |
| | No Algae | Synechococcus | Synechococcus |
| | Control | leopoliensis | leopoliensis (UTEX |
| | Acrylic Latex | (UTEX 2434) | 2434)/acrylic latex |
| Day 0 | 0.2919 | 6.7332 | 2.4436 |
| Day 4 | 0.0130 | 7.2495 | 2.7062 |
| Day 7 | −0.1834 | 8.2077 | 4.1709 |
| Day 10 | 0.0591 | 3.1317 | 4.7960 |
| Day 14 | −0.2407 | 8.5474 | 4.7091 |
| | | | Synechocystis |
| | | Synechocystis sp. | sp. (UTEX |
| | Acrylic Latex | (UTEX 2470) | 2470)/acrylic latex |
| Day 0 | 0.2919 | 4.7841 | 1.8786 |
| Day 4 | 0.0130 | 5.4703 | 1.8110 |
| Day 7 | −0.1834 | 5.3562 | 5.1632 |
| Day 10 | 0.0591 | 6.2687 | 3.1301 |
| Day 14 | −0.2407 | 8.5340 | 4.0717 |
| | | | Gloeocapsa alpicola |
| | | Gloeocapsa alpicola | (UTEX LB 1598)/ |
| | Acrylic Latex | (UTEX LB 1598) | acrylic latex |
| Day 0 | 0.0086 | 6.1537 | 4.3787 |
| Day 4 | 0.0591 | 4.6936 | 4.1770 |
| Day 7 | −0.2902 | 5.2808 | 3.7384 |
| | | Agmenellum | Agmenellum |
| | | quadruplicatum | quadruplicatum (UTEX |
| | Acrylic Latex | (UTEX 2268) | 2268)/acrylic latex |
| Day 0 | 0.0086 | 6.1153 | 1.7174 |
| Day 4 | 0.0591 | 6.3674 | 1.8967 |
| Day 7 | −0.2902 | 8.4501 | 1.2952 |

A similar study of Synechococcus leopoliensis (UTEX 2434; source UTEX) in polyvinyl acetate solution (Elmer's Products, Inc.) demonstrated a CO₂ capture rate of 4.15 mmol CO₂/hr/m².

### Example 8: Microorganism/Polymer Viability Assay After Different Lighting Conditions

A fresh culture of Synechococcus leopoliensis (UTEX 2434; source UTEX) which has been previously verified to capture CO₂ was centrifuged at 3000×g for 15 minutes, the supernatant was poured off and the pellet resuspended in remaining supernatant to produce a wet cell pellet. Alga/polymer coatings and controls shown at Table 17 below were prepared by mixing in microcentrifuge tubes.

TABLE 17
Alga/Polymer Coatings and Controls for Viability Assay

| | |
|---|---|
| “BG-11” | 80 μl BG-11 liquid medium |
| “2434 + BG-11” | 40 μl Synechococcus leopoliensis (UTEX |
| | 2434) pellet, 40 μl BG-11 liquid medium |
| | (described in Example 1) |
| “agar + BG-11” | 40 μl molten BG-11 agar, 40 μl BG-11 |
| | liquid medium |
| “2434 + agar” | 40 μl Synechococcus leopoliensis (UTEX |
| | 2434) pellet, 40μl molten BG-11 agar |
| “latex + BG-11” | 40 μl acrylic latex (Sherwin-Williams; pH |
| | 7), 40 μl BG-11 liquid medium |
| “2434 + latex” | 40 μl Synechococcus leopoliensis (UTEX |
| | 2434) pellet, 40 μl acrylic latex (Sherwin- |
| | Williams; pH 7) |
| “glue + BG-11” | 40 μl PVAc (Elmer's clear glue; Elmer's |
| | Products, Inc.), 40 μl BG-11 liquid |
| | medium |
| “2434 + glue” | 40 μl Synechococcus leopoliensis (UTEX |
| | 2434) pellet, 40 μl PVAc (Elmer's clear |
| | glue; Elmer's Products, Inc.) |

Ten microliters from each sample was dispensed into the wells of two duplicate 96-well microplates, which were left uncovered in a culture hood overnight to cure. The following day, fresh, identical samples were made in microcentrifuge tubes using the same materials (including the Synechococcus leopoliensis wet cell pellet), and 10 μl of each sample was dispensed into the wells of the two duplicate 96-well microplates. Immediately, 100 μl of BG-11 liquid medium was added to the wells with cured samples, and 90 μl of BG-11 was added to the wells with uncured samples. Twenty microliters of CellTiter 96 Aqueous One Solution Reagent (Promega, product #G358C, lot #0000330386) was added to each well. One plate was placed under the grow lights at 250 PAR illumination, and the other was placed on the bench top in a room with standard indoor lighting on and having the temperature set to 25° C., which is close to the temperature under the grow lights (26.1° C.). The plates were incubated for 4 hours. After incubation, the cells had settled to the bottoms of the wells, while the uncured latex samples were centrifuged at 13,000 rpm for 10 minutes in order to transfer supernatant. Sixty μl of the supernatant from each well was transferred to new wells, and the absorbance at 492 nm was read using the microplate reader. The percent increase in absorbance relative to controls lacking the cyanobacteria was determined and shown at Tables 18 to 21 below.

TABLE 18
Percent Absorbance Increase of Cured Alga/Polymer Coatings After
Grow Lights Incubation Relative to Alga Free Polymer Coating Controls

| Cured Alga/Polymer Coatings | % Increase |
|---|---|
| 2434 + BG-11 | 174.74 |
| 2434 + agar | 218.37 |
| 2434 + latex | 10.63 |
| 2434 + glue | 308.04 |

TABLE 19
Percent Absorbance Increase of Uncured Alga/Polymer Coatings After
Grow Lights Incubation Relative to Alga Free Polymer Coating Controls

| Cured Alga/Polymer Coatings | % Increase |
|---|---|
| 2434 + BG-11 | 911.57 |
| 2434 + agar | 460.16 |
| 2434 + latex | 326.61 |
| 2434 + glue | 458.85 |

TABLE 20
Percent Absorbance Increase of Cured Alga/Polymer Coatings After
Standard Indoor Lighting Incubation Relative to Alga Free Polymer
Coating Controls

| Cured Alga/Polymer Coatings | % Increase |
|---|---|
| 2434 + BG-11 | 19.79 |
| 2434 + agar | 23.35 |
| 2434 + latex | −4.08 |
| 2434 + glue | 259.33 |

TABLE 21
Percent Absorbance Increase of Uncured Alga/Polymer Coatings
After Standard Indoor Lighting Incubation Relative to Alga Free
Polymer Coating Controls

| Cured Alga/Polymer Coatings | % Increase |
|---|---|
| 2434 + BG-11 | 888.50 |
| 2434 + agar | 357.54 |
| 2434 + latex | 242.91 |
| 2434 + glue | 755.16 |

Cells generally grew better in the uncured coatings and under grow lights incubation. However, S. leopoliensis cells growth was reduced in the polyvinyl acetate under grow lights incubation; possibly a detrimental chemical reaction is being fostered by wavelengths in the grow lights.

### Example 9: Alga/Polymer Growth Assay

Various hydrogel and hydrophilic polymers [alginate, guar gum, both as described in Example 2; and crosslinked polyacrylamide (a hydrogel) in 1-2 mm particle size [Soil Moist™ granules, JRM Chemical Inc., 15663 NEO Parkway, Cleveland, Ohio 44128 U.S.A. (“JRM Chemical Inc.”)] were prepared by hydrating approximately 0.25-0.5 g of each polymer separately with about 5-10 g of deionized water in a scintillation vial. Approximately 1 mL of Spirulina sp. (Raw Living Spirulina) was mixed into each polymer to produce the algal/polymer mixtures. Optical 400× microscopic inspection found alga filaments fragmenting, though some intact fragments were observed in the alga/crosslinked polyacrylamide coating. It is contemplated that this visual assay may be correlated to CO₂ capture and/or cell viability assays for optimization of microorganism/polymer coating formulations.

### Example 10: Alga/Polymer Adhesion and Viability Assays

Hydrogel and hydrophilic polymers, shown at Table 22 below, were formulated with Spirulina sp. (Raw Living Spirulina) for visual adhesion and viability assays after coating PET beverage bottles and round glass scintillation vials.

TABLE 22
Polymers for Spirulina Visual Adhesion and Viability Assays

| Polymeric Material (type) | Source |
|---|---|
| Crosslinked sodium | Science Gone Fun ™ Super |
| polyacrylate | Asorbent Diaper Polymer; |
| (hydrogel) | Eco-Novelty Corp, |
| | Corporation Michigan, |
| | 5100 Kings Gate |
| | Way Bloomfield Hills, |
| | MI 48302 U.S.A. |
| | (“Eco-Novelty Corp.”) |
| Crosslinked polyacrylate | Soil Moist ™ granules; |
| (hydrogel) | JRM Chemical Inc. |
| Guar gum powder (food grade) | Best Food Ingredients. |
| Xanthan gum powder (food grade) | Anthony's Almonds |
| Carboxy methyl cellulose | Cake Supplies On Sale, |
| | 7706 Backlick Road, Unit I |
| | Springfield, VA 22150 U.S.A. |
| | (“Cake Supplies on Sale”) |
| Hydroxy ethyl cellulose | Akshar Chem India Ltd. |
| powder (“HEC”) | |
| Alginate molding mix (preformulated | LifeMold Alginate; |
| to gel upon addition of water; | EnvironMolds |
| ionically crosslinked hydrogel) | |

The water hydrogel and hydrophilic polymers were evaluated for alga compatibility after hydrating the polymers with deionized water and mixing in separate glass scintillation vials in accordance with Example 9, and incubating in a window facing the exterior environment; though a duplicate vial of Spirulina sp./crosslinked polyacrylate was prepared and placed under continuous grow lights illumination at 250 PAR. After 1 week of window lighting exposure, the guar gum, crosslinked polyacrylate, xanthan gum, and hydroxyl ethyl cellulose visually appeared remained green, indicating the Spirulina sp. remained viable. The crosslinked polyacrylate had large granules of hydrogel to allow light to pass through the sample, which was thought to be beneficial to the Spirulina sp. growth. After 16 hours of grow lights exposure, Spirulina sp. browning, an indication of cell death, was observed in Spirulina sp./polymer regions nearest the grow lights. By day 6 of grow lights exposure, the Spirulina sp./crosslinked polyacrylate samples remained green colored, which indicates living cells. Microscopic evaluation of samples was also conducted per Example 9 to evaluate cell fragmentation.

### Example 11: Polymer Coating Visual Adhesion Assay

Guar gum, a 1:1 mixture of guar gum:xanthan gum, and a crosslinked polyacrylate:guar gum 1:1 mixture were hydrated in accordance with Example 9 and used to coat beverage PET bottles internally by pouring the coating into each bottle and shaking and rotating each bottle until as much interior surface was coated as possible. This coating technique was typically used to coat PET beverage bottles in the Examples herein. The bottles were positioned vertically (cap on the bottom) overnight. Visual inspection demonstrated that the crosslinked polyacrylate/guar gum mixture had the best adherence to the bottle's surfaces.

Sodium alginate powder [Landor Trading Company, Landor Enterprises Inc., 1226 Vallamont Dr NW Williamsport, Pa. 17701 U.S.A. (“Landor Trading Company”)] and sodium alginate powder mixed 1:1 with guar gum polymer coatings were prepared to include calcium chloride dihydrate (“calcium chloride,” CAS no. 10035-04-8; Carolina Biological Supply Company) to evaluate calcium chloride induced crosslinking and subsequent adhesion properties after application to round PET bottles (same type of bottles as described about in Example 2). Approximately 30 ml of sodium alginate solution (2 wt % in reverse osmosis water) was used to coat the inside walls of each bottle. With each bottle being rotated about the bottle's long axis to prevent the mixture from settling to one side, a 5 wt % calcium chloride solution was added (50 mL) and the bottle was continually rotated slowly to allow the calcium solution to contact all of the sodium alginate material on the bottle wall. Tough gel coatings were produced by crosslinking, but the coatings delaminated from the bottle surfaces within minutes of forming crosslinks once the bottles were positioned upright (cap side up). The same procedure was repeated with a 1:1 sodium alginate:guar gum (2 wt % total with reverse osmosis water) coating material.

### Example 12: Alternative Carbon Dioxide Capture Device Geometries

This Example demonstrates that an algae-containing coating may be applied to various surfaces and geometric shapes to produce a carbon dioxide capture device.

Liquid medium for Spirulina sp. (“Spirulina Medium Recipe” UTEX) was prepared to the final concentration of the materials shown at Tables 23 to 25 below.

TABLE 23
P-IV Metal Solution

| Reagent Concentration | Source |
|---|---|
| 2 mM EDTA (disodium salt | Carolina Biological Supply Company |
| dihydrate) | item no. 86-1778 |
| 0.36 mM iron chloride | Sigma-Aldrich product no. 157740 |
| 0.21 mM manganese chloride | Sigma product no. M3634 |
| tetrahydrate | |
| 0.037 mM zinc chloride | Carolina Biological Supply Company |
| | item no. 89-9542 |
| 0.0084 mM cobalt chloride | Sigma-Aldrich product no. 255599 |
| hexahydrate | |
| 0.017 mM sodium molybdate | Sigma product no. M1651 |
| dihydrate | |

TABLE 24
Chu Micronutrient Solution

| Reagent Concentration | Source |
|---|---|
| 0.08 mM copper sulfate | Sigma Aldrich product no. 451657 |
| 0.15 mM zinc sulfate | Carolina Biological Supply Company |
| heptahydrate | item no. 89-9464 |
| 0.084 mM cobalt chloride | Sigma Aldrich product no. 255599 |
| hexahydrate | |
| 0.061 mM manganese chloride | Sigma product no. M3634 |
| tetrahydrate | |
| 0.052 mM sodium molybdate | Sigma product no. M1651 |
| dihydrate | |
| 10 mM boric acid | Carolina Biological Supply Company |
| | item no. 84-8450 |
| 0.13 mM EDTA (disodium salt | Carolina Biological Supply Company |
| dihydrate) | item no. 86-1778 |

TABLE 25
Spirulina sp. Liquid Medium

| | |
|---|---|
| Reagent | Source |

Solution 1: Prepared by Mixing the Following into 450 mL of
ddH₂O and Adjusting the Volume to be 500 mL

| | |
|---|---|
| 13.61 g sodium bicarbonate | Sigma product no. S5761 |
| 4.03 g sodium carbonate | Carolina Biological Supply Company |
| | item no. 88-8770 |
| 0.5 g potassium phosphate | Sigma Aldrich product |
| dibasic | no. P3786 |

Solution 2: Prepared by Mixing the Following into 450 mL of
ddH₂O and Adjusting the Volume to be 500 mL

| | |
|---|---|
| 2.5 g sodium nitrate, | Sigma Aldrich product no. S5506 |
| 1 g potassium sulfate, | Carolina Biological Supply item no. |
| | 88-4482 |
| 1 g sodium chloride, | Sigma Aldrich product no. S7653 |
| 0.2 g magnesium sulfate | Sigma Aldrich product no. M1880 |
| heptahydrate, | |
| 0.04 g calcium chloride | Carolina Biological Supply Company |
| dihydrate, | item no. 88-1800 |
| 6 mL P-IV Metal Solution | |
| 1 mL Chu Micronutrient | |
| Solution | |

Both solution 1 and solution 2 were autoclaved for 45 minutes and cooled. Then 1 mL of 0.1 mM vitamin B12 (Sigma product no. V6629) in 50 mM 4-(2-hydroxyethyl)-1-piperazineethanesulfonic acid (“HEPES”; Sigma product no. H3375) buffer, pH 7.8 was mixed into solution 2, and then solutions 1 and 2 mixed together.

For preparation of 1 L solid Spirulina sp. medium, solutions 1 and 2 were prepared the same except final volumes were 250 mL each. 15 g of agar was added to 500 mL of ddH₂O, and all solutions autoclaved separately for 45 minutes. After cooling to about 55° C., 1 mL of 0.1 mM vitamin B12 in 50 mM HEPES buffer, pH 7.8 was mixed into solution 2, and solutions 1 and 2 were mixed into the agar solution prior to pouring into Petri dishes to make Spirulina sp. medium agar plates.

Viscous polymers such as 10.29 g mildew free sealant [by weight, 30-60% limestone; 1-5% each of hydrotreated heavy paraffinic petroleum distillates, ethylene glycol, and titanium dioxide; 0.1-1% each of ammonium hydroxide, ethylene oxide-nonylphenol polymer, 3-iodo-2-propynyl butylcarbamate, and quartz; and less than 0.1% each of zinc oxide, aluminum hydroxide, formaldehyde, and ethyl alcohol; Sashco®, 10300 East 107^{th} Place, Brighton, Colo. 80601 U.S.A. (“Sashco®”)] were mixed with 2.29 g Spirulina medium and 0.58 g Spirulina sp. solid cell pellet by stirring by hand with a wooden tongue depressor. Due to the viscosity, the coating was not readily able to enter through the uncapped top, so the wide bottom of the bottles were cut off to produce an opening large enough to coat by smearing the sealant/Spirulina sp. on the interior surface of the bottle with a wooden tongue depressor Such a container (e.g., a bottle) having the bottom removed are known herein as a “bottomless container” (e.g., a bottomless bottle). Additionally, a bottle could now be placed top side first into the open bottom of another bottle, allowing a plurality of bottles to be stacked.

The removal of a bottle's bottom also allows for ease of dip coating, though both interior and exterior surfaces would be initially coated by this method. Spirulina sp. (5.5 g) and polyvinyl acetate (22 g of polyvinyl acetate described in Example 2) were mixed to prepare a coating that was dip coated onto a bottomless bottle, and a new bottom created by adhesive tape to prevent gas exchange while providing an opening to allow the Vernier Go Direct CO₂ sensor (Vernier Software & Technology) snuggly inserted to measure CO₂ capture.

The Spirulina sp./polyvinyl acetate coating was also used to coat the internal surfaces of a clear plastic cup by pouring the coating into the cup and rotating the cup; and flat plastic sheets by pouring the coating onto the sheet and brushing the surface to spread the coating. Intact spirals of Spirulina sp. cells, which is indicative of living, healthy cells, were observed under microscopic examination of this coating after being allowed to dry to a lower water content. A sample of such a Spirulina sp./polyvinyl acetate coated sheet was cut and placed into Spirulina sp. growth medium, after 2 weeks the Spirulina sp. was visibly alive.

Agar dissolved in deionized water (15 g agar/L) was poured into a square PET bottle and the agar was left on one side to gel. Polyvinyl acetate/Synechococcus leopoliensis (UTEX 2434) (source Promega) wet cell pellet was mixed in a 4:1 ratio by volume and approximately 1 g of the mixture was coated onto the solidified agar in the square PET bottle by pouring the Synechococcus leopoliensis coating onto the agar surface and rotating the bottle to spread the coating. The coated bottle was incubated under normal indoor lighting for 4 days had increased CO₂ capture relative to a polyvinyl acetate control lacking the alga.

### Example 13: Latex-Agar-Filter Paper/Cyanobacteria Carbon Dioxide Capture Device

An agar concentration was prepared between 1-4% w/v (10-20 g/L) in flasks by the following procedure. Agar (4 g in 100 mL BG-11 broth) was boiled to dissolve to prepare 4% BG-11 agar. Then, 50 mL 4% agar was mixed with 50 mL warm BG-11 to prepare 2% agar. Finally, 50 mL 2% agar was mixed with 50 mL warm BG-11 to prepare 1% agar. Each agar type (1%, 2%) was poured into 1 mL test tubes then transferred into 10 glass culture tubes that were loosely capped and autoclaved. The remaining agar was saved by autoclaving in loosely capped sealable Pyrex jars. After autoclaving, the tubes were placed in a 45° C. water bath and inspected to ensure some liquid agar remained. If not, new tubes were prepared using 2 mL of agar.

Acrylic latex was prepared by placing 15 mL latex (Sherwin-Williams, pH 7) in a 50 mL centrifuge tube, and warming in a 45° C. water bath for 15 minutes. Latex was fluid and not clumped for use.

In the 45° C. water bath, 2 mL, 1 mL and 0.5 mL of the acrylic latex was added to separate tubes of 4%, 2%, and 1% agar (9 tubes total prepared). At least 3 mL of cyanobacteria wet cell pellet was prepared in accordance with Example 1. Each agar-latex tube was taken out of the water bath individually and immediately had 250 cyanobacteria added, swirled to mix, and then quickly used to coat a separate filter paper disk before the agar-latex/cyanobacteria could solidify. Each coated disk was placed into a separate, uncovered Petri dish to cure overnight. The carbon dioxide capture in the filter paper/agar-latex/cyanobacteria carbon dioxide capture devices was then measured in a clear plastic chamber in accordance with Example 7.

### Example 14: Cellulose Production Assay

A 1% solution of calcofluor white cellulose stain [MP Biomedicals, LLC, 3 Hutton Center Drive, Suite 100, Santa Ana, Calif. 92707 U.S.A. (“MP Biomedicals”)] was prepared in distilled water. The 1% calcofluor white solution was applied with a dropper to the surface (2-3 drops) of materials comprising cellulose, such as filter paper, paper towel, and a plant leaf (after scraping surface); and allow to dry. The materials were then illuminated with a handheld 395 nm blacklight (Vansky 51 ultraviolet LED flashlight by Shenzhen Vansky Technology Co. Ltd., Shenzhen City, China [“Shenzhen Vansky Technology Co. Ltd.”]). A clear bluish fluorescence was observed in the presence of cellulose. It is contemplated this assay may be used to detect cellulose production in a carbon dioxide capture material or device.

### Example 15: Fluorescence Chlorophyll Detection Assay

Gloeocapsa alpicola (UTEX 1598) (source UTEX) was prepared as a wet cell pellet in accordance with Example 1, and 250 μL of wet pellet mixed in a microcentrifuge tube with 250 μL of a clear water-based acrylic latex (32.5% solids by weight, also having 1.0-2.5% wt % 2,2,4-trimethyl-1,3-pentanediol isobutyrate, 0.1-1.0 wt % 2,4,7,9-tetramethyl-5-decyne-4,7-diol, 0.1-1.0 wt % dipropylene glycol monomethyl ether, and less than 0.1 wt % of 5-chloro-2-methyl-4-isothiazolin-3-one mixture with 2-methyl-4-isothiazolin-3-one; Rust-Oleum® Painter's® Touch Ultracover Premium Latex Paint, clear gloss; source Rust-Oleum Corporation). A drawdown bar was used to apply a 6-mil thick wet film onto a polyester plastic substrate [Dura-Lar™ GRAFIX Clear 0.003 Dura-Lar Film, 9-Inch by 12-Inch; source Grafix Plastics, 5800 Pennsylvania Avenue, Maple Heights Ohio 44137 U.S.A. (“Grafix Plastics”)] and sheets were cured at ambient temperature overnight. As a control, the clear water-based acrylic latex without added Gloeocapsa alpicola (UTEX 1598) was applied and cured in the same manner. The cured samples were then cut into 1-inch squares and folded into 1-cm cuvettes for fluorescence analysis using a digital fluorometer [Sequoia Turner Model 450; source Barnstead Thermolyne Corporation, 14000 Unity Street, NW, Ramsey, Minn. 55303 U.S.A. (“Barnstead Thermolyne Corporation”)]. An excitation wavelength of 430 nm and an emission wavelength of 665 nm was used for fluorescence detection of chlorophyll. The cuvette containing the latex control was used to zero the relative fluorescence units (“RFU”) reading, then the Gloeocapsa alpicola (UTEX 1598)/latex coating was inserted for measurement. Three measurements were recorded (570 RFU, 550 RFU, and 440 RFU) for an average of 520 RFU for the Gloeocapsa alpicola (UTEX 1598) latex sample tested, indicating the presence of chlorophyll. It is contemplated that this assay may be used to detect photosynthetic cell viability and/or photosynthetic cell content in a carbon dioxide capture material or device.

### Example 16: Surface Area Densification

This Example demonstrates the manner in which carbon dioxide capture devices in accordance with one or more embodiments of the present invention enable densification of surface area providing photosynthesis relative to a terrestrial area occupied by the carbon dioxide capture facility. Table 26 discloses densification on a surface area basis provided for by carbon dioxide capture devices configured in accordance with one or more embodiment of the present invention.

TABLE 26
Surface Area Densification

| | Substrate Spacing Between 2.5 m2 | |
|---|---|---|
| | Coated Flat Panels | |
| | (1 m W × 2.5 m H × .004 m T) | |
| | 25 mm | 50 mm |
| No. of Substrate | 40 | 20 |
| Instances per 1-m² Plan- | | |
| View Unit Area | | |
| Total Coatable Surface | 100 m² | 50 m² |
| Area per M² Plan-View | | |
| Unit Area | | |
| Densification Factor | 100X | 50X |
| | Substrate Spacing Between | |
| | 6″ Dia. × 2.5 m H × .004 m T | |
| | Coated Cylindrical Tubes | |
| | (with 3″ interior nested sleeve) | |
| | 25 mm | 50 mm |
| No. of Substrate | 36 | 25 |
| Instances per 1-m² Plan- | | |
| View Unit Area | | |
| Total Coatable Surface | 43 m² (65 m²) | 30 m² (45 m²) |
| Area per M² Plan-View | | |
| Unit Area | | |
| Densification Factor | 43X (65X) | 30X (45X) |

Table 27 discloses densification on a volumetric basis provided for by carbon dioxide capture devices discussed above in reference to Table 26.

TABLE 27
Volumetric Densification

| | Substrate Spacing Between | |
|---|---|---|
| | 2.5 m2 Coated Flat Panels | |
| | (1 m W × 2.5 m H × ~.004 m T) | |
| | 25 mm | 50 mm |
| No. of Substrate | 40 | 20 |
| Instances per 1-m² Plan- | | |
| View Unit Area | | |
| Total Occupied Volume | 1 m³ | 0.05 m² |
| per 2.5 m³ Space | | |
| Volumetric Utilization | 16% | 8% |
| in 2.5 m³ Space | | |
| | Substrate Spacing Between | |
| | 6″ Dia. × 2.5 m H × 0.004 T | |
| | Coated Cylindrical Tubes | |
| | (with 3″ interior nested sleeve of same H and T) | |
| | 12 mm | 25 mm |
| No. of Substrate | 36 | 25 |
| Instances per 1-m² Plan- | | |
| View Unit Area | | |
| Total Occupied Volume | ~0.17 m³ (0.25 m³) | 0.12 m² (0.18 m³) |
| per 2.5 m³ Space | | |
| Volumetric Utilization | 6.8% (10.0%) | 4.8% (7.2%) |
| in 2.5 m³ Space | | |

It is disclosed herein that geometric structures can be used to further enhance volumetric densification. For example, standing ribs, arcuate surfaces and the like can be used to provide additional coatable surface area of a substrate within a given volumetric space. In one specific example, for substrates spaced apart by about 25 mm (e.g., 1-inch), a surface area multiplier (over a flat panel surface area) of about 2×-3× can be achieved through use of standing ribs having a height about equal to space lateral spacing between planar substrates (e.g., 1-inch planar surface between thin (e.g., 0.125″ thick) 1-inch tall ribs), where the planar surface between ribs and at least one side surface of the rib is coated with a carbon dioxide capture coating composition in accordance with one or more embodiments of the present invention. In another specific example, for substrates spaced apart by about 25 mm (e.g., 1-inch), a surface area multiplier (over a flat panel surface area) of about 1.5× can be achieved through use of a semi-circular surface having a radius about equal to space lateral spacing between planar substrates (e.g., repeating 1-inch radius semi-circular surfaces), where the planar surface between ribs and at least one side surface of the rib is coated with a carbon dioxide capture coating composition in accordance with one or more embodiments of the present invention.

### Example 17: Carbon Dioxide Capture Rate

This Example demonstrates the manner in which surface area densification provided for in Example 16 provides a corresponding multiplication of provides for carbon dioxide capture rates markedly greater than that attainable by mature forests (e.g., via afforestation and/or reforestation practices). Table 28 discloses per-acre carbon dioxide capture rates achievable via coated substrate carbon dioxide capture rates achieved with coating compositions in accordance with one or more embodiments of the present invention in combination with disclosed surface area densification provided for by such coatings.

TABLE 28
Per-Acre Carbon Dioxide Capture Rates

| | Annualized Per-Acre CO₂ Capture Rate | |
|---|---|---|
| | (Metric-Tons/Year/Acre) for 0.25 to 3.00 | |
| | mmol/m2/hr nominal coated | |
| | substrate CO₂ capture rate | |
| | 100X Densification | 50X Densification |
| 3 m Facility Height | 40-468 | 20-234 |
| 12 m Facility Height | 117-1404 | 59-702 |
| 30 m Facility Height | 390-4681 | 195-2340 |

### Example 18: Fossil Fuel Powered Vehicle Carbon Dioxide Offset

This Example demonstrates the manner in which carbon dioxide capture facilities in accordance with one or more embodiments of the present invention provide for carbon dioxide offset of fossil fuel powered motor vehicles. It is reported that a typical passenger vehicle emits, on average, 4.6 metric tons of carbon dioxide per year. (U.S. Environmental Protection Agency, Office of Transportation and Air Quality, Greenhouse Gas Emissions from a Typical Passenger Vehicle, EPA 420-F-18-008, Washington, D.C., March 2018, Page 2.) It is reported that an average mature forest captures about 4.0 metric tons of carbon dioxide per year per acre—i.e., about one (1) car's worth of annual carbon dioxide emissions. (Congressional Research Service, Library of Congress, U.S. Tree Planting for Carbon Sequestration, Washington D.C., May 2009, Page 2.) Table 29 discloses fossil fuel powered vehicle carbon dioxide offset information for the annualized per-acre carbon dioxide captures of Example 17.

TABLE 29
Fossil Fuel Powered Passenger Vehicle CO₂ Offset

| | Annualized Per-Acre CO₂ Offset | |
|---|---|---|
| | no. of vehicles offset | |
| | 100X Densification | 50X Densification |
| 3 m Facility Height | ~9-102 | ~4-51 |
| 12 m Facility Height | ~25-305 | ~17-153 |
| 30 m Facility Height | ~85-1018 | ~42-509 |

### Example 19: Ranges

To provide a description that is both concise and clear, various examples of ranges have been identified herein. Any range cited herein includes any and all sub-ranges and specific values within the cited range, this example provides specific numeric values for use within any cited range that may be used for an integer, intermediate range(s), subrange(s), combinations of range(s) and individual value(s) within a cited range, including in the claims. Examples of specific values (e.g., %, kDa, ° C., μm, kg/L, Ku) that can be within a cited range include 0.000001, 0.000002, 0.000003, 0.000004, 0.000005, 0.000006, 0.000007, 0.000008, 0.000009, 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, 2.16, 2.17, 2.18, 2.19, 2.20, 2.21, 2.22, 2.23, 2.24, 2.25, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.10, 99.20, 99.30, 99.40, 99.50, 99.60, 99.70, 99.80, 99.90, 99.91, 99.92, 99.93, 99.94, 99.95, 99.96, 99.97, 99.98, 99.99, 99.999, 99.9999, 99.99999, 99.999999, 99.9999999, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5250, 5500, 5750, 6000, 6250, 6500, 6750, 7000, 7250, 7500, 7750, 8000, 8250, 8500, 8750, 9000, 9250, 9500, 9750, 10,000, 25,000, 50,000, 75,000, 100,000, 250,000, 500,000, 1,000,000, or more. Additional examples of the use of this definition to specify sub-ranges are given herein. For example, a cited range of 25,000 to 100,000 would include specific values of 50,000 and/or 75,000, as well as sub-ranges such as 25,000 to 50,000, 25,000 to 75,000, 50,000 to 100,000, 50,000 to 75,000, and/or 75,000 to 100,000. In another example, the range 875 to 1200 would include values such as 910, 930, etc. as well as sub-ranges such as 940 to 950, 890 to 1150, etc.

In embodiments wherein a value or range is denoted in exponent form, both the integer and the exponent values are included. For example, a range of 1.0×10¹⁷ to 2.5×10⁻⁷, would include a description for a sub-range such as 1.24×10¹⁷ to 8.7×10¹¹.

However, general sub-ranges for each type of unit (e.g., %, kDa, ° C., kg/L, Ku) are contemplated, as the values typically found within a particular type of unit are of a sub-range of the integers described above. For example, integers typically found within a cited percentage range, as applicable, include 0.000001% to 100%. Examples of values that can be within a cited molecular mass range in kilo Daltons (“kDa”) as applicable for many coating components include 0.50 kDa to 110 kDa. Examples of values that can be within a cited temperature range in degrees Celsius (“° C.”) as may be applicable in the arts of a polymeric material (e.g., a coating) include −10° C. to 500° C. Examples of values that can be within a thickness range in micrometers (“μm”) as may be applicable to coating and/or film thickness upon a surface include 1 μm to 2000 μm. Examples of values that can be within a cited density range in kilograms per liter (“kg/L”) as may be applicable in the arts of a material formulation (e.g., a polymeric material) include 0.50 kg/L to 20 kDa. Examples of values that can be within a cited shear rate range in Krebs Units (“Ku”), as may be applicable in the arts of a material formulation, include 20 Ku to 300 Ku.

### Example 20: Nitrogen Fixation Coatings

To demonstrate nitrogen fixation and carbon dioxide capture in algae-based coatings, the product of nitrogen fixation activity, ammonium, as well as chlorophyll A and carbon dioxide capture, were measured in coatings containing nitrogen fixing algae.

BG-11 media was prepared as previously described in Example 1. BG-11 without nitrate [“BG-11(−N)”] was prepared by adding 10 mL each of the stock solutions described in Example 1 of K₂HPO₄, MgSO₄.7H₂O, CaCl₂) 2H₂O, citric acid.H₂O, ferric ammonium citrate, Na₂EDTA.2H₂O, and Na₂CO₃ in order to about 900 mL of dH₂O while stirring, and then adding 1 mL of the BG-11 Trace Metals Solution of Example 1. Additional dH₂O was added as needed to the 900 mL dH₂O and stock solutions mixture to produce a final volume of 1 liter. The BG-11(−N) medium was covered, autoclaved for 45 minutes, and then stored at room temperature. To produce BG-11 or BG-11(−N) media having L-methionine sulfoximine (“MSX”; Acros Organics, part of Thermo Fisher Scientific, Janssen Pharmaceuticalaan 3A, Geel 2440, Belgium), 10 mM MSX stock solution in dH₂O was added to the BG-11 or BG-11(−N) media to achieve 10 μM, 50 μM, and 100 μM concentrations following autoclaving.

BG-11 agar was prepared as previously described in Example 1. To produce BG-11(−N) agar medium, stock solutions of the same reagents in the same amounts for BG-11(−N) liquid medium were instead added in the same order to 400 mL of stirred dH₂O. Additional dH₂O was added as needed to produce a final volume of 500 mL. To a separate container of 500 mL dH₂O, 15 grams of agar was added, and both containers covered, autoclaved for 45 minutes, and placed in a water bath to cool to about 45-55° C. Then 1 mL of the sodium thiosulfate pentahydrate stock solution of Example 1 was mixed thoroughly into the agar solution; and the two solutions mixed thoroughly to create BG-11(−N) agar medium. The BG-11(−N) agar medium was stored at refrigerated temperatures. To produce BG-11 media having MSX (Acros Organics), 10 mM MSX stock solution in dH₂O was added to the BG-11 or BG-11(−N) media to achieve 10 μM, 50 μM, and 100 μM concentrations prior to mixing the two solutions.

Algae wet cell pellet (“WCP”) was prepared by growing either Anabaena sp. (UTEX 2576), Anabaena variabilis (UTEX B 377), or Nostoc muscorum (UTEX 2209) (cultures were obtained from the “UTEX” Culture Collection of Algae Austin, Tex.) in 350-400 mL of BG-11 in one-liter flasks under 70 PAR growth lights with gentle shaking at 100 rpm until the OD₅₄₀ was about 0.3-0.5. About 400 mL culture was centrifuged at 3000× gravity for 15 minutes to pellet the cells. The supernatant was discarded and the cell pellet resuspended in 10 mL BG-11(−N), and then transferred into a 15 mL centrifuge tube. The suspension was centrifuged at 3000 rpm for 5 minutes, and the supernatant discarded and resuspended in 10 mL BG-11(−N), and this process of centrifuging at 3000 rpm for 5 minutes and resuspension repeated 2 additional times to wash nitrate from the pelleted cells. The cells were resuspended in about 4 mL BG-11(−N) for an OD₅₄₀ of 1 or a volume proportionate to the OD₅₄₀ [e.g., cultures that had an OD₅₄₀ around 0.5 (half the target of OD₅₄₀=1) were resuspended in 2 mL BG-11(−N)] to produce the final WCP.

Nine microfuge tubes of coating mixtures were prepared by mixing 9×0.1 mL of the WCP into 9×14 mL BG-11 (−N). Nine additional microfuge tubes of coating mixtures were prepared having 0.1 mL of the WCP, adding 10 mM MSX (Acros Organics) stock solution in dH₂O to a final concentration of 10 μM MSX, and mixing into 9×14 mL BG-11 (−N) having 10 μM MSX. Two grams of alginate (LifeMold Alginate, EnvironMolds, LLC), then 4 g of 2 wt % xanthan gum (Anthony's Almonds) in BG-11(−N) (xanthan gum prepared by slow stirring in accordance with Example 6) were quickly mixed into a separate tube of WCP (mixing the coatings one at a time). Coating controls having 14 mL BG-11(−N) without algae were similarly prepared in triplicate. Each algae or control coating preparation was poured into Petri dishes (100 mm×15 mm, Fisher Scientific catalogue no. FB0875712) and allowed to cure. The day of coating preparation was designated as day 0 herein, and the next day designated day 1, etc.

To measure ammonia production by the coatings, every 4 or 7 days (depending on the sample), 5 mL phosphate buffered saline (“PBS”; prepared as described in Molecular Cloning: A Laboratory Manual, 3rd ed., Vols 1, 2 and 3, J. F. Sambrook and D. W. Russell, ed., Cold Spring Harbor Laboratory Press, 2001) was dispensed into the Petri dish and the Petri dish was placed on a rocker at room temperature for 5 minutes. A pipet was used to transfer the 5 mL of the wash from the Petri dish to a 15 mL tube. The tube was centrifuged at 3000 rpm for 5 minutes to pellet debris, and 50 μL of each supernatant transferred to wells in a 96-well microplate. Then 50 μL of each standard (0 (PBS), 1, 2.5, 5, 25, 125, 250, and 500 μM NH₄Cl) was dispensed into wells of the same 96-well microplate. The Amplite™ Colorimetric Quantitation Kit (AAT Bioquest, Inc. 520 Mercury Dr, Sunnyvale, Calif. 94085 U.S.A.) was used to measure ammonium concentration in each well by dispensing 50 μL of Buffer I from the kit into each well and incubating at room temperature (approximately 21° C. for 5 minutes), then dispensing 50 of Buffer II from the kit into each well and incubating at room temperature for 1 hour, and then measuring the absorbance at 660 nm. One set of coatings were washed on days 4 and 8, while another set of coatings were washed on day 7. The amounts of ammonium extracted from the coatings are shown at the Tables 30 to 32 below, with ammonium being produced in coatings from both Nostoc muscorum (day 7) and Anabaena sp., demonstrating that algae cells in coatings on solid surfaces can fix atmospheric nitrogen.

TABLE 30
Ammonium Extracted from the Alginate with
Xanthan Gum/Nostoc muscorum (UTEX 2209) Coated Device

| Day | Average μmol NH₄/m² |
|---|---|
| 4 | −0.4138 |
| 7 | 1.9475 |
| 8 | −1.8581 |

TABLE 31
Ammonium Extracted from the Alginate with
Xanthan Gum/Anabaena sp. UTEX 2576 Coated Device

| Day | Average μmol NH₄/m² |
|---|---|
| 4 | 3.1504 |
| 7 | 4.5833 |
| 8 | 0.3687 |

TABLE 32
Ammonium Extracted from the
Alginate with Xanthan Gum/No Algae Control Coated Device

| Day | Average μmol NH₄/m² |
|---|---|
| 4 | 0.0564 |
| 7 | 0.0579 |
| 8 | 0.0507 |

To measure chlorophyll A from dissolved coatings, a glass tube was used to cut a disc of about 1.54 cm² from each coating that was just washed, and the coating disc was transferred to a 15 mL tube. Then 2.5 mL 0.3 M EDTA (Himedia® Laboratories, located at 23, Vadhani Industrial Estate, L.B.S. Marg, Mumbai—400 086, India) and 1.25 mL 0.5 M sodium citrate (Himedia® Laboratories) was added to each tube, and the tubes vortexed until the coating was dissolved, well mixed and a homogenous suspension. Then 1 mL of each suspension was transferred to separate microfuge tubes, each tube centrifuged at 13,300 rpm for 1 minute, and 900 μL of the supernatant was discarded. Nine hundred microliters of methanol was added to each tube that was then vortexed to mix. The tubes were placed on a rocker in the dark for 5 minutes, then centrifuged at 13,300 rpm for 1 minute. The absorbance at 665 nm for the supernatant was measured, and the valued multiplied by 12.7 (Meeks and Castenholz. Arch. Mikrobiol. 78:25-41, 1974) to determine the concentration of chlorophyll A in μg/mL. The chlorophyll A extracted amounts from the coatings are shown at the Tables 33 to 34 below, with chlorophyll A being produced in coatings from both Nostoc muscorum and Anabaena sp., demonstrating that algae cells in coatings on solid surfaces can survive and be photosynthetically active.

TABLE 33
Chlorophyll A Extracted from the Alginate with Xanthan
Gum/Nostoc muscorum (UTEX 2209) Coated Device

| Day | Average μg Chlorophyll A/m² |
|---|---|
| 1 | 93.9743 |
| 4 | 156.1987 |
| 7 | 313.2864 |
| 8 | 221.5387 |

TABLE 34
Chlorophyll A Extracted from the Alginate with Xanthan
Gum/Anabaena sp. UTEX 2576 Coated Device

| Day | Average μg Chlorophyll A/m² |
|---|---|
| 1 | 84.4777 |
| 4 | 220.6908 |
| 7 | 404.5290 |
| 8 | 344.9467 |

To measure the CO₂ fixation for algae-based coatings, the same procedure as above for preparing wet cell pellet and coatings was used to prepare Petri dishes having algae coatings. The Petri dishes (with lids removed) were place in a sealed Rubbermaid® container (Brilliance Pantry Airtight Food Storage Container, 7.8 Cup, from Rubbermaid® 5, 4475 S Fulton Pkwy, Atlanta, Ga. 30349) with a grommet-sealed hole, and a Vernier® Go Direct CO₂ sensor [Vernier Software & Technology] placed in the grommet-sealed hole. The sealed container was placed under the 250 PAR grow lights and CO₂ capture (or release) measured for 1 hour, and the slope of the line plotted against time to determine the rate of CO₂ fixation (or respiration). CO₂ captured amounts are shown at the Tables 35 to 37 below, with carbon dioxide being captured in coatings from both Nostoc muscorum (days 1 and 3) and Anabaena sp., demonstrating that algae cells in coatings on solid surfaces can capture carbon dioxide.

TABLE 35
CO₂ Capture of the Alginate with Xanthan
Gum/Nostoc muscorum (UTEX 2209) Coated Device

| Day | Average mmol CO₂/m²/hr Capture |
|---|---|
| 1 | 0.5029 |
| 3 | 0.3455 |
| 6 | −0.2333 |
| 9 | −0.3990 |

TABLE 36
CO₂ Capture of the Alginate with Xanthan
Gum/Anabaena sp. UTEX 2576 Coated Device

| Day | Average mmol CO₂/m²/hr Capture |
|---|---|
| 1 | 0.4523 |
| 3 | 0.2500 |
| 6 | 0.1437 |
| 9 | 0.6757 |

TABLE 37
CO₂ Capture of the Alginate with Xanthan
Gum/No Algae Control Coated Device

| Day | Average mmol CO₂/m²/hr Capture |
|---|---|
| 1 | 0.0167 |
| 3 | −0.0608 |
| 6 | −0.4189 |
| 9 | −1.1858 |

Cyanobacteria capable of fixing gaseous (e.g., atmospheric) nitrogen do so when preferred sources of environmental nitrogen, namely ammonia and nitrate, are not available. When such nitrogen sources are available, genes involved in gaseous nitrogen-fixation are not expressed (Flores et al. 1999, p. 463-477. In G. A. Peschek, W. Löffelhardt, and G. Schmetterer (ed.), The phototrophic prokaryotes. Plenum Publishing Corporation, New York, N.Y.; Huang et al. Microbiology 145: 743-753, 1999; Herrero et al. J. Bacteriol. 183: 411-425 2001). Ammonia limitation increases expression of genes involved in utilizing alternative nitrogen sources and assimilation of nitrogen, all of which are repressed by the restoration of ammonium levels.

Ammonium is incorporated into organic compounds primarily through the glutamine synthase-glutamate synthase cycle that utilizes the carbon skeleton of 2-oxoglutarate (Wolk et al. J. Biol. Chem. 251: 5027-5034, 1976; Flores and Herrero, p. 463-477. In G. A. Peschek, W. Löffelhardt, and G. Schmetterer (ed.), The phototrophic prokaryotes. Plenum Publishing Corporation, New York, N.Y., 1994; Muro-Pastor and Florencio, Eur. J. Biochem. 203: 99-105, 1992; Muro-Pastor and Florencio, J. Bacteriol. 176: 2718-2726, 1994). Nitrogen fixing cyanobacteria cultures treated with the glutamine synthase inhibitor MSX have the repressive effect of ammonium on the nitrogen starvation response inhibited, and are expected to have increased expression of the genes involved in gaseous (e.g., atmospheric) nitrogen fixation, and thus expected to have enhanced gaseous nitrogen fixation rates and produce greater quantities of ammonium as a product of nitrogen fixation, and/or have an excess of the carbon skeleton 2-oxoglutarate since glutamine synthase is unable to incorporate the ammonium into this skeleton. This excess of 2-oxoglutarate may inhibit upstream pathways leading to 2-oxoglutarate formation, including carbon fixation (Flores and Herrero, p. 463-477. In G. A. Peschek, W. Löffelhardt, and G. Schmetterer (ed.), The phototrophic prokaryotes. Plenum Publishing Corporation, New York, N.Y., 1994; Forchhammer and Tandeau de Marsac, J. Bacteriol. 176: 84-91, 1994; Forchhammer, p. 549-553. In G. A. Peschek, W. Löffelhardt, and G. Schmetterer (ed.), The phototrophic prokaryotes. Plenum Publishing Corporation, New York, N.Y., 1999, Lee et al., J. Bacteriol. 181: 2697-1702, 1999).

To immobilize the cells in the hydrogel-based coating, 100 μL of the WCPs was dissolved into 14 mL of BG-11(−N) or 14 mL BG-11(−N) with 10 μM MSX. Two grams of alginate (LifeMold Alginate, EnvironMolds, LLC) was quickly and vigorously stirred into the cell-media mixture, followed by 2% xanthan gum in BG-11(−N). The mixture was poured into a sterilized 8.5 cm diameter Petri dish and allowed to cure at room temperature. Petri dishes were incubated on the lab benchtop under about 7 PAR fluorescent lighting at 21° C. with 12-hour light and dark cycles. The plates were washed with 5 mL of PBS at various times, and the PBS collected and analyzed for ammonia content and chlorophyll A content of the coating as described above, and the results shown at Tables 38 to 41 below. The CO₂ capture rates were measured for the Petri dishes as described above, and the results shown at Tables 42 to 43 below.

TABLE 38
Ammonium Extracted from the Alginate with Xanthan
Gum/Nostoc muscorum (UTEX 2209) Coated Device Having MSX

| Day | Average μmol NH₄/m² |
|---|---|
| 4 | 6.3307 |
| 7 | 8.9064 |
| 8 | 2.3932 |

TABLE 39
Ammonium Extracted from the Alginate with Xanthan
Gum/Anabaena sp. UTEX 2576 Coated Device Having MSX

| Day | Average μmol NH₄/m² |
|---|---|
| 4 | 3.1431 |
| 7 | 8.1994 |
| 8 | 2.4205 |

TABLE 40
Chlorophyll A Extracted from the Alginate with Xanthan Gum/
Nostoc muscorum (UTEX 2209) Coated Device Having MSX

| Day | Average μg Chlorophyll A/m² |
|---|---|
| 1 | 79.5167 |
| 4 | 211.4354 |
| 7 | 217.5833 |
| 8 | 154.5273 |

TABLE 41

| Chlorophyll A Extracted from | |
|---|---|
| the Alginate with Xanthan Gum/ | |
| Anabaena sp. UTEX 2576 Coated | |
| Device Having MSX | |
| | Average μg |
| Day | Chlorophyll A/m² |
| 1 | 52.8396 |
| 4 | 149.6786 |
| 7 | 223.3839 |
| 8 | 63.0847 |

TABLE 42

| CO₂ Capture of the Alginate | |
|---|---|
| with Xanthan Gum/Nostoc muscorum | |
| (UTEX 2209) Coated Device Having MSX | |
| | Average mmol |
| Day | CO₂/m²/hr Capture |
| 1 | 0.5312 |
| 3 | 0.3392 |
| 6 | −0.2294 |
| 9 | −0.4434 |

TABLE 43

| CO₂ Capture of the Alginate | |
|---|---|
| with Xanthan Gum/Anabaena sp. UTEX | |
| 2576 Coated Device Having MSX | |
| | Average mmol |
| Day | CO₂/m²/hr Capture |
| 1 | 0.2635 |
| 3 | 0.3223 |
| 6 | −0.3344 |
| 9 | −0.3881 |

To screen for MSX concentrations sufficient to induce ammonia release from cyanobacterial cells, 50 μL of WCP was extruded onto triplicate 15 mm diameter nylon filters with a pore size of 0.45 μm (Membrane Solutions, LLC., 20021 80th Avenue South, C-4, Kent, Wash. 98032, U.S.A.). After air-drying for 5 minutes, the filters were placed on BG-11, BG-11(−N), or BG-11(−N) having 10 μM, 50 μM or 100 μM MSX agar media. Cyanobacteria immobilized on filters were incubated on the lab benchtop under about 7 PAR fluorescent lighting at 21° C. with 12-hour light and dark cycles. The cells were washed from the filters using 1 mL of PBS at various times and the amounts of ammonia and chlorophyll A in this wash were quantified as described above. The ammonia content and chlorophyll A content obtained from the cyanobacteria filters are shown at Tables 44 to 46 below.

TABLE 44
Ammonium and Chlorophyll A Extracted from the Nylon Filters-
Anabaena variabilis (UTEX B 377) Coated Device Having MSX

| | | MSX | | Average μg |
|---|---|---|---|---|
| | | Concentration | Average μmol | Chlorophyll |
| Day | Media | (μM) | NH₄/m² | A/m² |
| 1 | BG-11 | 0 | 1.6776 | 2.0743 |
| 4 | BG-11 | 0 | −0.0459 | 2.6221 |
| 7 | BG-11 | 0 | 0.1271 | 4.5796 |
| 11 | BG-11 | 0 | −0.2709 | 5.3505 |
| 1 | BG-11 | 10 | 10.9493 | 0.9601 |
| 4 | BG-11 | 10 | 5.9974 | 1.4249 |
| 7 | BG-11 | 10 | 2.3046 | 1.9427 |
| 11 | BG-11 | 10 | 0.1083 | 3.2398 |
| 1 | BG-11 | 50 | 5.1837 | 1.4254 |
| 4 | BG-11 | 50 | 4.4776 | 1.6383 |
| 7 | BG-11 | 50 | 5.8401 | 1.4889 |
| 11 | BG-11 | 50 | 1.7424 | 1.4969 |
| 1 | BG-11 | 100 | 3.9229 | 2.9261 |
| 4 | BG-11 | 100 | 3.7140 | 2.2407 |
| 7 | BG-11 | 100 | 4.7138 | 2.1810 |
| 11 | BG-11 | 100 | 2.9156 | 1.9550 |
| 1 | BG-11 (−N) | 0 | 1.0222 | 2.9642 |
| 4 | BG-11 (−N) | 0 | 0.7327 | 3.0260 |
| 7 | BG-11 (−N) | 0 | 4.1241 | 4.7329 |
| 11 | BG-11 (−N) | 0 | −0.2334 | 5.6452 |
| 1 | BG-11 (−N) | 10 | 11.1634 | 0.8035 |
| 4 | BG-11 (−N) | 10 | 1.7053 | 1.9647 |
| 7 | BG-11 (−N) | 10 | 1.2348 | 3.0548 |
| 11 | BG-11 (−N) | 10 | 1.7993 | 3.1805 |
| 1 | BG-11 (−N) | 50 | 7.1621 | 1.3572 |
| 4 | BG-11 (−N) | 50 | 2.1466 | 2.1010 |
| 7 | BG-11 (−N) | 50 | 2.8878 | 1.9414 |
| 11 | BG-11 (−N) | 50 | 3.2455 | 2.0735 |
| 1 | BG-11 (−N) | 100 | 6.6300 | 1.6163 |
| 4 | BG-11 (−N) | 100 | 4.6691 | 2.4680 |
| 7 | BG-11 (−N) | 100 | 3.0314 | 2.3245 |
| 11 | BG-11 (−N) | 100 | 3.9301 | 1.8936 |

TABLE 45
Ammonium and Chlorophyll A Extracted from the Nylon Filters-
Nostoc muscorum (UTEX 2209) Coated Device Having MSX

| | | MSX | | |
|---|---|---|---|---|
| | | Concentration | Average μmol | Average μg |
| Day | Media | (μM) | NH₄/m² | Chlorophyll A/m² |
| 1 | BG-11 | 0 | 0.6136 | 10.5545 |
| 4 | BG-11 | 0 | 1.5178 | 6.7594 |
| 7 | BG-11 | 0 | 0.2566 | 12.9608 |
| 11 | BG-11 | 0 | 0.2181 | 21.7259 |
| 1 | BG-11 | 10 | 1.8711 | 6.4778 |
| 4 | BG-11 | 10 | 3.3900 | 6.3538 |
| 7 | BG-11 | 10 | 4.7717 | 4.5347 |
| 11 | BG-11 | 10 | 5.2837 | 6.7183 |
| 1 | BG-11 | 50 | 1.5752 | 6.9545 |
| 4 | BG-11 | 50 | 2.8559 | 4.3976 |
| 7 | BG-11 | 50 | 1.7680 | 6.4541 |
| 11 | BG-11 | 50 | 5.7436 | 5.1808 |
| 1 | BG-11 | 100 | 1.6456 | 7.1243 |
| 4 | BG-11 | 100 | 2.8963 | 4.5851 |
| 7 | BG-11 | 100 | 1.4513 | 5.7459 |
| 11 | BG-11 | 100 | 2.0211 | 4.9856 |
| 1 | BG-11 (−N) | 0 | 0.5698 | 7.4469 |
| 4 | BG-11 (−N) | 0 | 0.2599 | 6.7852 |
| 7 | BG-11 (−N) | 0 | 0.0216 | 7.8799 |
| 11 | BG-11 (−N) | 0 | 0.4677 | 10.7133 |
| 1 | BG-11 (−N) | 10 | 1.5540 | 8.2597 |
| 4 | BG-11 (−N) | 10 | 2.2041 | 5.4221 |
| 7 | BG-11 (−N) | 10 | 3.3835 | 5.7573 |
| 11 | BG-11 (−N) | 10 | 1.7726 | 6.9935 |
| 1 | BG-11 (−N) | 50 | 1.2804 | 8.8045 |
| 4 | BG-11 (−N) | 50 | 2.6180 | 5.2536 |
| 7 | BG-11 (−N) | 50 | 2.5772 | 7.7004 |
| 11 | BG-11 (−N) | 50 | 2.4657 | 6.0638 |
| 1 | BG-11 (−N) | 100 | 1.4630 | 9.1262 |
| 4 | BG-11 (−N) | 100 | 2.8796 | 5.7556 |
| 7 | BG-11 (−N) | 100 | 2.4383 | 5.9881 |
| 11 | BG-11 (−N) | 100 | 3.1487 | 5.3785 |

TABLE 46
Ammonium and Chlorophyll A Extracted from the Nylon Filters-
Anabaena sp. (UTEX 2576) Coated Device Having MSX

| | | MSX | | |
|---|---|---|---|---|
| | | Concentration | Average μmol | Average μg |
| Day | Media | (μM) | NH₄/m² | Chlorophyll A/m² |
| 1 | BG-11 | 0 | 1.5543 | 7.3745 |
| 4 | BG-11 | 0 | 0.2279 | 8.2715 |
| 7 | BG-11 | 0 | 0.2201 | 12.6708 |
| 11 | BG-11 | 0 | 0.3306 | 16.1641 |
| 1 | BG-11 | 10 | 3.7159 | 6.8732 |
| 4 | BG-11 | 10 | 7.6092 | 6.3826 |
| 7 | BG-11 | 10 | 6.0237 | 6.5756 |
| 11 | BG-11 | 10 | 5.9932 | 6.8982 |
| 1 | BG-11 | 50 | 4.1344 | 7.0206 |
| 4 | BG-11 | 50 | 2.3127 | 5.2074 |
| 7 | BG-11 | 50 | 2.5452 | 6.3390 |
| 11 | BG-11 | 50 | 2.4980 | 6.3415 |
| 1 | BG-11 | 100 | 3.2091 | 7.3254 |
| 4 | BG-11 | 100 | 2.0785 | 6.2708 |
| 7 | BG-11 | 100 | 2.6427 | 5.4919 |
| 11 | BG-11 | 100 | 4.4668 | 4.3900 |
| 1 | BG-11 (−N) | 0 | 0.5808 | 7.9735 |
| 4 | BG-11 (−N) | 0 | 0.0482 | 8.3913 |
| 7 | BG-11 (−N) | 0 | 0.4282 | 8.6673 |
| 11 | BG-11 (−N) | 0 | 0.2594 | 11.9228 |
| 1 | BG-11 (−N) | 10 | 3.5232 | 5.8839 |
| 4 | BG-11 (−N) | 10 | 3.9363 | 4.4209 |
| 7 | BG-11 (−N) | 10 | 2.0285 | 6.5773 |
| 11 | BG-11 (−N) | 10 | 5.4506 | 6.4702 |
| 1 | BG-11 (−N) | 50 | 2.3225 | 7.3685 |
| 4 | BG-11 (−N) | 50 | 1.9074 | 5.55117 |
| 7 | BG-11 (−N) | 50 | 3.7252 | 3.683 |
| 11 | BG-11 (−N) | 50 | 5.3047 | 3.9895 |
| 1 | BG-11 (−N) | 100 | 4.5907 | 5.5224 |
| 4 | BG-11 (−N) | 100 | 2.3296 | 4.7714 |
| 7 | BG-11 (−N) | 100 | 2.4147 | 5.7789 |
| 11 | BG-11 (−N) | 100 | 6.3080 | 4.3146 |

Ammonia levels in Anabaena sp. (UTEX 2576) in BG-11(−N) media was similar to BG-11 after 11 days, while NH₃ levels in Nostoc muscorum (UTEX 2209) in BG-11 (−N) media was about half that of Nostoc muscorum (UTEX 2209) in BG-11. Both Anabaena sp. (UTEX 2576) and Anabaena variabilis (UTEX B 377) had an initial increase in NH₃ levels, which decreased more in Anabaena variabilis (UTEX B 377). The production of ammonia was initially greater for cultures in BG-11, which may have been caused by uptake of nitrates from the media and conversion to NH₃. Except for Anabaena variabilis (UTEX B 377) particularly in BG-11 (−N), the algae release of ammonia tended to increase over time when treated with MSX relative to controls, indicating that Anabaena variabilis (UTEX B 377) may be partly or fully insensitive to MSX. Alga growth tended to be slower on BG-11(−N) compared to BG-11, except for Anabaena variabilis (UTEX B 377). Except for Anabaena variabilis (UTEX B 377), algae growth on 10 μM MSX was generally stationary, as opposed to higher concentrations of MSX wherein chlorophyll A production tended to be reduced.

### Example 21: Nitrogen Fixation Coatings and Cyanobacterial Filament

To demonstrate nitrogen fixation in algae-based coatings, the product of nitrogen fixation activity, ammonium, as well as chlorophyll A, cyanobacterial filament, and carbon dioxide capture, were measured in coatings containing nitrogen fixing algae.

BG-11 media was prepared as previously described in Example 1. Algae wet cell pellet was prepared (Day 1) by growing Anabaena variabilis (UTEX B 377) (cultures were obtained from the UTEX Culture Collection of Algae Austin, Tex.) in 60 mL of BG-11 in a 250 mL flask under 70 PAR grow lights with gentle shaking at 100 rpm until the OD₅₄₀ was 0.4580. The culture was centrifuged at 3000× gravity in a swinging bucket rotor for 5 minutes to pellet the cells. The supernatant was discarded and the cell pellet resuspended in 20 mL BG-11.

To measure initial chlorophyll A content, one milliliter of the resuspended culture was each placed in 3 microfuge tubes and the tubes centrifuged at 13,300 rpm for 1 minute. Nine hundred microliters of the supernatant was removed from each tube, and 900 μL of methanol was added to the tubes, which were then vortexed to mix. The tubes were placed on a rocker in the dark for 5 minutes, then centrifuged at 13,300 rpm for minute. The absorbance at 665 nm for the supernatant was measured, and the valued multiplied by 12.7 (Meeks and Castenholz. Arch. Mikrobiol. 78:25-41, 1974) to determine the concentration of chlorophyll A in μg/mL. The chlorophyll A extracted amounts from the culture are shown at the Table 47 below.

TABLE 47

| Chlorophyll A Extracted from | | | |
|---|---|---|---|
| Anabaena variabilis Cultures | | | |
| | | Average | Average Chlorophyll A |
| Tube | A₆₆₅ | A₆₆₅ | (μg/mL) |
| 1 | 0.3507 | 0.3639 | 4.6 |
| 2 | 0.3266 | | |
| 3 | 0.4143 | | |

Alga coatings were prepared by vigorously stirring 2 grams of alginate (LifeMold Alginate, EnvironMolds, LLC) into 14 mL of the resuspended culture, then stirring 4 g of 2 wt % xanthan gum (Anthony's Almonds) in BG-11 (xanthan gum prepared by slow stirring in accordance with Example 6). An alga free control controls having 14 mL water without algae was similarly prepared. The algae or control coating preparation was poured into Petri dishes (100 mm×15 mm, Fisher Scientific catalogue no. FB0875712) and allowed to cure at room temperature.

To measure chlorophyll A from dissolved coatings, a glass tube was used to cut discs in triplicate of about 4 cm² from each 56 cm² Petri dish coating area, so that the cell content of the each disk was equivalent to 1 mL of the original liquid culture, and the weight of each disc was about 1 gram each. Each disc was placed in separate glass centrifuge tubes, and 6 mL 0.3 M EDTA (Himedia® Laboratories) and 3 mL 0.5 M sodium citrate (Himedia® Laboratories) was added to each tube, and the tubes vortexed periodically until the coating was dissolved, well mixed and a homogenous suspension. The tubes were centrifuged at 3000 rpm for 5 minutes, and the supernatant was discarded. The remaining cell pellets were about 400 μL each, and to make a 90% methanol solution 3.6 mL of methanol was added to each pellet, which was vortexed to resuspend the pellet and then vortexed intermittently for 5 minutes in order to keep the pellet suspended. The resulting suspension would be a 4-fold dilution of the original 1 mL cell suspension. The tubes of suspensions were then centrifuged at 3000 rpm for 5 minutes. The absorbance at 665 nm for the supernatant was measured, and the valued multiplied by 12.7 (Meeks and Castenholz. Arch. Mikrobiol. 78:25-41, 1974) and then multiplied by 4 to match the dilution in order to determine the concentration of chlorophyll A in μg/mL of the original 1 mL cell suspension. The chlorophyll A extracted amounts from the coatings are shown at the Table 48 below.

TABLE 48
Chlorophyll A Extracted from Anabaena variabilis Coatings

| Coating | | | Avg. | | −control | Chlorophyll |
|---|---|---|---|---|---|---|
| Samples | Tube | A₆₆₅ | A₆₆₅ | x4 | A₆₆₅ | a (μg/mL) |
| Control | 1 | 0.0062 | 0.0073 | — | 0 | — |
| | 2 | 0.0175 | | | | |
| | 3 | −0.0018 | | | | |
| +UTEX | 1 | 0.0676 | 0.0816 | 0.3263 | 0.3190 | 4.05 |
| B 377 | 2 | 0.0586 | | | | |
| | 3 | 0.1185 | | | | |

The adjusted chlorophyll A content from the coating corresponding to 1 mL from the original culture, the recovery of cells from the coating was (4.05/4.6)*100%=88% recovery. Since the chlorophyll A contents shown in Table 48 originated from 4 cm² disk, the chlorophyll A content of the coating was, on average, 1.0125 μg chlorophyll A/cm².

The Petri dishes with coatings were stored on benchtop in lab at room temperature. To recover cyanobacterial filaments from the coatings, the next day, a glass tube was used to cut discs of about 4 cm² from each Petri dish coating area. Each disc was placed in separate glass centrifuge tube, and 6 mL 0.3 M EDTA (Himedia® Laboratories) and 3 mL 0.5 M sodium citrate (Himedia® Laboratories) was added to each tube, and the tubes vortexed periodically until the coating was dissolved, well mixed and a homogenous suspension. The tubes were centrifuged at 3000 rpm for 5 minutes, and the supernatant was discarded. The resulting supernatant was poured off, and the pellet (cells+cellulose) was resuspended in the remaining liquid. Twenty-five microliters was dropped onto a positively-charged slide and allowed to sit at room temperature for about 1 hour. The slide was washed with PBS (prepared in accordance with Example 20), then visualized under the compound light microscope. Recovered filaments from coatings showed defined heterocysts that are contemplated to be quantifiable.

On Day 51 the chlorophyll A content was again measured for the coated Petri dishes that had previously been assayed by disc removal, and a chlorophyll A content of 23.45 ug/cm² was measured. These Petri dishes had their average CO₂ uptake measured in accordance with Example 20, and a CO₂ uptake rate was 2.025 mmol CO₂/hr/m².

Three milliliters of the 20 mL resuspended cell pellet on the day of preparation described above was centrifuged at 3000 rpm for 5 minutes in a swinging bucket rotor, and most of the supernatant was discarded. The cells were resuspended in the remaining supernatant and transferred to a microfuge tube, which was centrifuged at 13,300 rpm for 1 minute. The supernatant was drawn off so that the final volume was about 60 μL. Fifty microliters was pipetted onto the surface of a 15 mm diameter filter with 0.45 μm pore size (Membrane Solutions, LLC). The filter was allowed about 5 minutes to dry, after which it was placed onto a BG-11 with 100 μM MSX agar plate (agar prepared in accordance with Example 20) for 24 hours. The cells were washed from the filter into a microfuge tube using 1.25 mL of PBS, which was divided for chlorophyll and ammonia analysis.

Done in triplicate, five hundred microliters of the filter wash was transferred to a microfuge tube and centrifuged at 13,300 rpm for 1 minute, and 450 μL ( 1/9^{th} of total volume) of the supernatant was discarded. The pellet was resuspended in the remaining liquid, and 450 μL of methanol was added. The tube was vortexed to resuspend the cells, rocked gently in the dark for 5 minutes. The tube was centrifuged at 13,300 rpm for 1 minute, and the absorbance at 665 nm of the supernatant was measured. The absorbance was multiplied by 12.7 to get the chlorophyll A concentration in μg/mL. The chlorophyll A extracted amounts from the coatings are shown at the Table 49 below.

TABLE 49

| Chlorophyll A Extracted from | | | |
|---|---|---|---|
| Anabaena variabilis Nylon Filters | | | |
| | | | Average |
| | | Chlorophyll A | Chlorophyll |
| Tube | A₆₆₅ | (μg/mL) | A (μg/mL) |
| 1 | 0.6702 | 8.51 | 8.68 |
| 2 | 0.6670 | 8.47 | |
| 3 | 0.7139 | 9.07 | |

The remaining wash was centrifuged at 13,300 rpm for 1 minute, and 3×50 μL of the supernatant was transferred to 3 wells in a 96-well microplate. Wells containing 50 μL of PBS (0 μM NH₄⁺), 1.13 μM NH₄⁺, 4.1 μM NH₄⁺, 12.3 μM NH₄⁺, 37 μM NH₄⁺, 111 μM NH₄⁺, 333 μM NH₄⁺, and 1000 μM NH₄⁺ standards were also prepared. Using the Amplite™ Colorimetric Quantitation Kit (AAT Bioquest, Inc.), 50 μL of Assay Buffer I was added to each well and incubated at room temperature for 5 minutes. Fifty microliters of Assay Buffer II was added to each well and incubated at room temperature for 1 hour. The absorbance of each well was measured at 630 nm using the microplate reader as well as at 660 nm using the Beckman-Coulter spectrophotometer, with a PBS sample used as blank. The amounts of ammonia of the standards and extracted from the coatings are shown at the Tables 50 to 52 below.

TABLE 50
NH₄Cl standards

| μM | | |
|---|---|---|
| NH₄Cl | A₆₃₀ | A₆₆₀ |
| 0 | 0.0000 | 0.0000 |
| 1.13 | −0.0056 | 0.0085 |
| 4.1 | −0.0186 | 0.0177 |
| 12.3 | 0.0048 | 0.0580 |
| 37 | 0.0285 | 0.1714 |
| 111 | 0.1666 | 0.5977 |
| 333 | 0.5198 | 1.6937 |
| 1000 | 1.5301 | 3.8741 |

TABLE 51
Ammonia levels in samples, measuring A₆₃₀

| | | | Avg | |
|---|---|---|---|---|
| | | NH₄⁺ | NH₄⁺ | |
| Sample | A₆₃₀ | (μM) | (μM) | Std. dev |
| UTEX 377 #1 | 0.0018 | 1.2 | 6.4 | 8.3 |
| UTEX 377 #2 | 0.024 | 16 | | |
| UTEX 377 #3 | 0.003 | 2 | | |

| | |
|---|---|
| nmol NH₄⁺/μg | 0.74 |
| Chlorophyll A | |

TABLE 52
Ammonia levels in samples, measuring A₆₆₀

| | | | Avg | |
|---|---|---|---|---|
| | | NH₄⁺ | NH₄⁺ | |
| Sample | A₆₆₀ | (μM) | (μM) | Std. dev |
| UTEX 377 #1 | 0.0540 | 10.6 | 10.9 | 0.4 |
| UTEX 377 #2 | 0.0552 | 10.8 | | |
| UTEX 377 #3 | 0.0578 | 11.3 | | |

| | |
|---|---|
| nmol NH₄⁺/μg | 1.26 |
| Chlorophyll A | |

Measuring absorbance at 660 nm showed higher sensitivity (detected down to 1.13 as opposed to 12.3 μM for 630 nm), and a lower standard deviation among the samples (SD=0.4 compared to SD=8.3 for 630 nm). In addition, the value for nmol NH₄⁺/μg chlorophyll A was closer to the expected value of 2 (comparing 1.26 using 660 nm, and 0.74 using 630 nm).

### Example 22: Carbon Dioxide Capture in a Planar Device

To demonstrate carbon dioxide capture in a planar device using coatings having a higher xanthan gum concentration, alga coatings were placed in Petri dishes and carbon dioxide capture measured.

BG-11 media was prepared as previously described in Example 1. Algae wet cell pellet was prepared (Day 1) by growing Synechococcus leopoliensis UTCC100 NS:cat. (source UTEX Culture Collection of Algae) in accordance with Example 1. Two wt % xanthan gum (Anthony's Almonds) was prepared by slow stirring in BG-11 in accordance with Example 6.

For each Petri dish's (100 mm×15 mm, Fisher Scientific catalogue no. FB0875712) coating, 0.93 grams of alginate (LifeMold Alginate, EnvironMolds, LLC) was by vigorously stirred into either 10.67 ml algal culture or 5.4 ml algal culture and 5.4 ml BG-11, then 8.33 g of 2 wt % xanthan gum in BG-11 was stirred in. The combined components were mixed by hand using a tongue depressor and poured into a 100 mm Petri dish and allowed to cure at room temperature. The Petri dishes were then placed under grow lights at 100 PAR overnight (“O.N.”).

On Day 2, single Petri dishes were monitored for CO₂ capture by placing with lids removed in a sealed Rubbermaid® container (Brilliance Pantry Airtight Food Storage Container, 7.8 Cup, from Rubbermaid® 5, 4475 S Fulton Pkwy, Atlanta, Ga. 30349) with a grommet-sealed hole, and a Vernier® Go Direct CO₂ sensor [Vernier Software & Technology] placed in the grommet-sealed hole. The sealed container was placed under the 250 PAR grow lights and CO₂ capture (or release) measured for 1 hour, and the slope of the line plotted against time to determine the rate of CO₂ fixation (or respiration). The Petri dishes were then left under 100 PAR light exposure overnight again for a total of 2 days under Petri dishes under 100 PAR for 2 days, and then the Petri dishes were placed on a benchtop under ambient indoor lighting during the day. Control Petri dishes were not illuminated for the 2^{nd} day by 100 PAR and instead placed on the benchtop after initial CO₂ capture (or release) measurement for 1 hour.

CO₂ capture was monitored for 1 hour under 100 PAR light at day 14, and then immediately tested again for 1 hour, and compared control Petri dishes left on benchtop and exposed to the 100 PAR light for 1 day rather than 2 days.

TABLE 53
CO₂ Capture of the Alginate with Xanthan Gum/
Synechococcus leopoliensis UTCC100 NS: cat. Coated Device

| | Coating and | Days | | | Average |
|---|---|---|---|---|---|
| | Previous | O.N. | Hour of | | mmol |
| | Lighting | at 100 | Measurement: | CO₂ Uptake | CO₂/m²/hr |
| Day | Conditions | PAR | 1^{st} or 2^{nd} | Illumination | Capture |
| 2 | 5.4 mL Alga | 1 | 1^{st} | 100 PAR | 3.1481 |
| 2 | 10.56 mL Alga | 1 | 1^{st} | 100 PAR | 3.2324 |
| 14 | 5.4 mL Alga | 1 | 1^{st} | 100 PAR | 0.2099 |
| 14 | 5.4 mL Alga | 1 | 2^{nd} | 100 PAR | 0.6518 |
| 14 | 5.4 mL Alga | 2 | 1^{st} | 100 PAR | 0.8340 |
| 14 | 5.4 mL Alga | 2 | 2^{nd} | 100 PAR | 1.5986 |
| 14 | 10.67 mL Alga | 1 | 1^{st} | 100 PAR | 0.6510 |
| 14 | 10.67 mL Alga | 1 | 2^{nd} | 100 PAR | 1.3006 |
| 14 | 10.67 mL Alga | 2 | 1^{st} | 100 PAR | −0.5856 |
| 14 | 10.67 mL Alga | 2 | 2^{nd} | 100 PAR | 0.51864 |

CO₂ capture rates of over 3 mmol CO₂/hr/m² were measured on day 1. On day 14, the 2-day 100 PAR illuminated Petri dishes with 5.4 ml algae had greater CO₂ capture rates than the 1-day exposure Petri dishes; however, the Petri dishes with 10.67 ml algae had lower CO₂ capture rates than the 1 day exposure Petri dishes.

### Example 23: Long-Term Carbon Dioxide Capture by Carbon Dioxide Capture Devices

To demonstrate carbon dioxide capture over months, carbon dioxide capture devices were constructed and carbon dioxide capture measured over months.

Carbon dioxide capture devices were constructed in accordance with Example 6, with the modifications that 96 round 500 mL PET beverage bottles (The Coca Cola Company) and 24 square sided 500 mL PET beverage bottles (Platinum Supplies LLC, 5 Baila Boulevard, Lakewood, N.J. 08701 U.S.A.) were coated per day for having Synechococcus leopoliensis UTCC100 NS:abAc strain UTCC 100 (source UTEX Culture Collection of Algae). The Synechococcus leopoliensis UTCC100 NS:abAc strain had been genetically modified to have and express the cellulose synthesis genes (acsABAC) from Gluconacetobacter hansenii. Control carbon capture devices that were constructed included 24 round 500 mL PET beverage bottles and 6 square sided 500 mL PET beverage bottles coated with coatings having no alga. Immediately after construction, four round beverage containers and 1 square sided beverage container having alga coating were placed in a plastic sleeve (Cleartec Packaging product no. PRT00107) so that the sleeve had 5 loosely stacked coated beverage bottles (carbon dioxide capture devices) with the coated square sided beverage container in the middle position, and the sleeve placed into a slot of a support structure, an architect's blueprint holder (Model #3089 of Safco Products Company), referred herein as a “rack.” All bottles had their uncapped openings facing upward, and the sleeves were uncapped. The process of coated bottles in sleeve construction was repeated until all 24 slots of the rack were filled, and the sleeves numbered sequentially from 1 to 24. Six control sleeves with the no alga coated bottles were also constructed and placed in a control rack, and each sleeve numbered sequentially from 25 to 30. Another rack of alga coating coated bottles in sleeves and 6 sleeves of no-alga control bottles were constructed the same way on the next day, with the control sleeves placed in the control rack. On the third day another 24 sleeves having alga coating coated bottles in another rack were constructed, and 6 sleeves having no-alga coating coated bottles were prepared and placed in the control rack, with the modification that the round PET beverage bottle at the bottom of each sleeve was replaced with a stack of 14 covered but vented, 60 mm rounded edge Petri dishes (Part no. T3305 from Tritech Research, Inc., 2961 Veteran Avenue, Los Angeles, Calif. 90064, U.S.A.) having 10 grams of coating poured into each dish to create carbon dioxide capture devices wherein the coating was on horizontal surfaces to contrast to the bottles wherein the coating was primarily on vertical surfaces. To support each stack of Petri dishes as they had smaller diameters than the sleeve, each stack was placed in a narrower plastic sleeve (Cleartec Packaging product no. PRT00103) that fit into the sleeve, with the narrower plastic sleeve cut to only be as tall as each stack of Petri dishes.

Four weeks later, 24 sleeves having alga coating coated bottles and Petri dishes and 6 control sleeve having no alga coating in the carbon dioxide capture devices were prepared and placed into another rack and 6 slots of the control rack as described above for the day 3 rack construction, with the modification that the alga mixed into the coating was Synechococcus leopoliensis UTCC100 NS:cat. (source UTEX Culture Collection of Algae). Synechococcus leopoliensis UTCC100 NS:cat. is a strain that does not have cellulose synthase genes, but was genetically modified to have the chloramphenicol acteyltransferase gene that provides resistance to chloramphenicol. All racks of sleeves were maintained under ambient indoor lighting during the day at room temperature.

The day after, and every seven days after each rack and set of control sleeves were constructed, a sample of square sided bottles typically comprising 3 alga coated bottles and 1 control bottle was measured for carbon dioxide capture for one hour by placing a Vernier® Go Direct CO₂ sensor (Vernier Software & Technology) at the opening of each bottle and wrapping the sensor and bottle ends with Parafilm™ (Bemis Company, Inc.) to prevent gas exchange with the atmosphere. For racks having Petri dishes, 1 to 3 stacks of 14 alga coating containing Petri dishes and 1 control stack also were measured for carbon dioxide capture for 1 hour by placing a Petri dish stack in a 250 mL clear plastic chamber (Rubbermaid® brilliance pantry airtight food storage container 7.8 cup volume; from Rubbermaid® Home Products, 3415 E 12th Ave, Winfield, Kans. 67156, U.S.A.) modified to have an opening for connecting to a Vernier Go Direct CO₂ sensor (Vernier Software & Technology). All CO₂ capture measurements were conducted under 250 PAR grow light illumination. Typically different bottles and stacks of Petri dishes had carbon dioxide capture measured every seven days to evaluate carbon dioxide capture from carbon dioxide capture devices located in different areas of each rack or set of control sleeves. Initially no carbon dioxide capture was measured, which may be due to the alga cells respiring as they divide and grow until a static phase of growth is reached wherein net carbon dioxide capture begins. When carbon dioxide capture was detected, a sample set of the same bottles or Petri dish stacks (e.g., 3 bottles) that contained visibly healthy alga due to a green color in the coatings were selected to have carbon dioxide capture measured each week. No Petri dish stack achieved carbon dioxide capture by day 77 or day 42 for the strain of alga genetically modified to produce cellulose and the control strain of alga, respectively, though measurements continue. The days when carbon dioxide capture was detected and sleeves where the sample set of same bottles were located in the racks are shown at Tables 54 and 55 below.

TABLE 54
CO₂ Capture of the Alginate with Xanthan Gum/
Synechococcus leopoliensis UTCC100 NS: cat. Coated Device

| Sleeve | | |
|---|---|---|
| Where | | |
| Bottle | | |
| Obtained | Day | Mmol CO₂/m²/hr Capture |
| 9 | 21 | 0.1338 |
| 20 | 28 | 0.0778 |
| 20 | 29 | 0.0793 |
| 20 | 35 | 0.1374 |
| 20 | 42 | 0.2024 |
| 21 | 29 | 0.1232 |
| 21 | 35 | 0.1448 |
| 21 | 42 | 0.2048 |
| 22 | 29 | 0.1606 |
| 22 | 35 | 0.0303 |
| 22 | 42 | 0.0957 |

TABLE 55
CO₂ Capture of the Alginate with Xanthan
Gum/Synechococcus leopoliensis UTCC100
NS: abΔc strain UTCC 100 Coated Device

| Sleeve | | |
|---|---|---|
| Where | | |
| Bottle | | |
| Obtained | Day | Mmol CO₂/m²/hr Capture |
| 4 | 63 | 0.1307 |
| 4 | 70 | 0.1022 |
| 4 | 77 | 0.1672 |

The disclosures herein provide a detailed understanding of the nature and function of embodiments of the present invention. Detailed descriptions of embodiments of the present invention are provided herein, as well as, the best mode of carrying out and employing embodiments of the present invention. It will be readily appreciated that embodiments of the present invention are well adapted to carry out and obtain the ends and features mentioned as well as those inherent therein. It is to be understood, however, that the present invention may be embodied in various forms. Therefore, specific details disclosed herein are not to be interpreted as limiting, but rather as a basis for the claims and as a representative basis for teaching to employ the present invention in virtually any appropriately detailed system, structure or manner. Other features will be readily apparent from the following detailed description; specific examples and claims; and various changes, substitutions, other uses and modifications that may be made to the embodiments disclosed herein without departing from the scope and spirit of the invention or as defined by the scope of the appended claims.

It should be understood that biological cell(s), coating(s), paint(s), polymeric material(s), material formulation(s), compound(s), method(s), procedure(s), and technique(s) described herein are presently representative of various embodiments. These techniques are intended to be exemplary, are given by way of illustration only, and are not intended as limitations on the scope. All patents and publications mentioned in this specification are herein incorporated by reference to the same extent as if each individual publication was specifically and individually indicated to be incorporated by reference. U.S. patent application Ser. Nos. 14/097,128, 12/696,651, 12/474,921, 12/243,755, and 10/884,355 and U.S. Provisional Patent Application Nos. 62/626,044, 61/148,502, 61/057,705, 61/058,025; 60/485,234, 60/976,676, and 60/409,102, each of which are specifically incorporated herein by reference.

As used herein other than the claims, the terms “a,” “an,” “the,” and/or “said” means one or more. As used herein in the claim(s), when used in conjunction with the words “comprise,” “comprises” and/or “comprising,” the words “a,” “an,” “the,” and/or “said” may mean one or more than one. As used herein and in the claims, the terms “having,” “has,” “is,” “have,” “including,” “includes,” and/or “include” has the same meaning as “comprising,” “comprises,” and “comprise.” As used herein and in the claims “another” may mean at least a second or more. As used herein and in the claims, “about” refers to any inherent measurement error or a rounding of digits for a value (e.g., a measured value, calculated value such as a ratio), and thus the term “about” may be used with any value and/or range.

The phrase “a combination thereof” “a mixture thereof” and such like following a listing, the use of “and/or” as part of a listing, a listing in a table, the use of “etc.” as part of a listing, the phrase “such as,” and/or a listing within brackets with “e.g.,” or “i.e.,′ refers to any combination (e.g., any sub-set) of a set of listed components, and combinations and/or mixtures of related species and/or embodiments described herein though not directly placed in such a listing are also contemplated. For example, compositions described as a polymer suitable for use in a coating described in different sections of the specification may be claimed individually and/or as a combination, as they are part of the same genera of preservative (e.g., a coating preservative). Such related and/or like genera(s), sub-genera(s), specie(s), and/or embodiment(s) described herein are contemplated both in the form of an individual component that may be claimed, as well as a mixture and/or a combination that may be described in the claims as, for example, “at least one selected from,” “a mixture thereof” and/or “a combination thereof.”

In various embodiments described herein, exemplary values are specified as a range, and all intermediate range(s), subrange(s), combination(s) of range(s) and individual value(s) (e.g., an integer, a fraction, etc.) within a cited range are contemplated and included herein. For example, citation of a range “0.03% to 0.07%” provides specific values within the cited range, such as, for example, 0.03%, 0.04%, 0.05%, 0.06%, and 0.07%, as well as various combinations of such specific values, such as, for example, 0.03%, 0.06% and 0.07%, 0.04% and 0.06%, and/or 0.05% and 0.07%, as well as sub-ranges such as 0.03% to 0.05%, 0.04% to 0.07%, and/or 0.04% to 0.06%, etc. In another example, a range of “0.0001% to 20.0%” provides specific values and sub-ranges such as “8.5%,” and “11.3 to 18.9%.” Example 16 above provides additional descriptions of specific numeric values within any cited range that may be used for an integer, intermediate range(s), subrange(s), combinations of range(s) and individual value(s) within a cited range, including in the claims.

Some terms often have different meanings for different material types and/or uses being described, and the meaning applicable to the material should be applied as appropriate in the context, as understood in the applicable art. For example, in the context of a polymeric material, other than a coating, a “film” (“polymeric film”) of a polymeric material refers to a planar form (i.e., a large width and large length relative to thickness) capable of being flexed, creased without cracking, folded, or a combination thereof, while being self-supporting (e.g., a plastic wrap), though such a film may also be treated with a surface treatment (e.g., coated with a coating). A polymeric film comprises from about 5 μm to about 250 μm thick (e.g., about 10 μm to about 180 μm thick), while a plastic sheet (“sheeting”) refers to a planar form having a thickness of about 250 μm to about 250 mm thick. Thus, a “film,” for example, in the plastic art being described and/or claimed in the context of a plastic differs in composition, meaning, manufacture process, function and/or purpose than a “film” in a coating (e.g., a paint) art. In another example, a “cell” in a biological art refers to the smallest unit of living matter, while a “cell” in a polymeric material art (e.g., a plastic art, an elastomer art) refers to a void in a polymeric material to produce a solid foam material (e.g., a plastic foam, an elastomer foam material). A surface comprises the outer layer of any solid object. The term “substrate,” in the context of a coating, may be synonymous with the term “surface.” However, as “substrate” has a different meaning in the art of enzymology, a chemical that undergoes an accelerated chemical reaction upon contact with an enzyme, the term “surface” may be preferentially used herein for clarity. In such instances, the appropriate definition and/or meaning for the term should be applied in accordance with the context of the term's use in light of the present disclosures.

What is claimed is:

1. A carbon dioxide capture apparatus, comprising:
a plurality of substrates each arranged and supported in a spaced-apart relationship with each adjacent one of the substrates;
wherein said spaced-apart substrates jointly define a plan view area occupied thereby;
wherein each of the substrates has one or more surfaces coated with a layer of carbon-dioxide capture coating composition; and
wherein the substrate inhibits the transmission of water through a thickness thereof and along a length and width thereof.

2. The carbon dioxide capture apparatus of claim 1 wherein:
said one or more coatable surfaces of each of the substrates define a respective coatable surface area thereof;
a total coatable surface area of the carbon dioxide capture apparatus is equal to an aggregation of the coatable surface area of all of the substrates; and
the total coatable surface area of the carbon dioxide capture apparatus is at least 40 times the plan view area.

3. The carbon dioxide capture apparatus of claim 1 wherein the substrates extend vertically in the plan view.

4. The carbon dioxide capture apparatus of claim 1 wherein the substrate is made from a material that is light transmissive.

5. The carbon dioxide capture apparatus of claim 1 wherein each of the carbon dioxide capture devices is approximately evenly spaced away from each adjacent one of the carbon dioxide capture devices.

6. The carbon dioxide capture apparatus of claim 1 wherein the substrate of each of the carbon dioxide capture devices is generally flat.

7. The carbon dioxide capture apparatus of claim 6 wherein the substrates extend vertically in the plan view.

8. The carbon dioxide capture apparatus of claim 1 wherein the carbon-dioxide capture coating composition is adhered to the surface respectively coated thereon.

9. The carbon dioxide capture apparatus of claim 8 wherein the substrates extend vertically in the plan view.

10. The carbon dioxide capture apparatus of claim 8 wherein the substrate is made from a material that is light transmissive.

11. The carbon dioxide capture apparatus of claim 8 wherein the substrate of each of the carbon dioxide capture devices is generally flat.

12. The carbon dioxide capture apparatus of claim 1 wherein:
the substrate of each of the carbon dioxide capture devices is in the form of an elongated tubular body;
each elongated tubular body has a different cross-sectional size than each other elongated tubular body; and
a particular one of the elongated tubular bodies has nested therein all of the other elongated tubular bodies having a cross-sectional size smaller than the cross-sectional size of the particular one of the elongated tubular bodies.

13. The carbon dioxide capture apparatus of claim 12 wherein the carbon-dioxide capture coating composition is adhered to the surface respectively coated thereon.

14. The carbon dioxide capture apparatus of claim 13 wherein the substrates extend vertically in the plan view.

15. The carbon dioxide capture apparatus of claim 13 wherein the substrate is made from a material that is light transmissive.

16. A carbon dioxide capture apparatus, comprising:
a device retaining structure having an interior space; and
a plurality of carbon dioxide capture devices within the interior space, wherein each of the carbon dioxide capture devices includes a substrate having one or more surfaces thereof coated with carbon-dioxide capture coating composition, wherein each of the carbon dioxide capture devices is spaced away from each adjacent one of the carbon dioxide capture devices to provide an airspace between opposing faces thereof therebetween and wherein the substrate inhibits the transmission of water through a thickness thereof and along a length and width thereof.

17. The carbon dioxide capture apparatus of claim 16 wherein the one or more surfaces of each of the carbon dioxide capture devices extends vertically within the interior space of the device retaining structure.

18. The carbon dioxide capture apparatus of claim 16 wherein the substrate is made from a material that is light transmissive.

19. The carbon dioxide capture apparatus of claim 16 wherein each of the carbon dioxide capture devices is approximately evenly spaced away from each adjacent one of the carbon dioxide capture devices.

20. The carbon dioxide capture apparatus of claim 16 wherein the carbon-dioxide capture coating composition is adhered to the surface respectively coated thereon.

21. The carbon dioxide capture apparatus of claim 16 wherein:
the substrate is a container has a fluid-receiving space having includes a closed end portion and an open end portion; and
the one or more surfaces of the substrate coated with the carbon-dioxide capture coating composition is within the fluid-receiving space.

22. The carbon dioxide capture apparatus of claim 21 wherein:
the container has a body portion and a neck portion attached to the body portion; and
the body portion and the neck portion jointly define the fluid-receiving space.

23. The carbon dioxide capture apparatus of claim 22 wherein the substrate is made from a material that is light transmissive.

24. The carbon dioxide capture apparatus of claim 21 wherein the container is a single-use beverage bottle.

25. The carbon dioxide capture apparatus of claim 24 wherein the substrate is made from a material that is light transmissive.

26. The carbon dioxide capture apparatus of claim 16 wherein:
the substrate of each of the carbon dioxide capture devices is in the form of an elongated tubular body;
each elongated tubular body has a different cross-sectional size than each other elongated tubular body; and
a particular one of the elongated tubular bodies has nested therein all of the other elongated tubular bodies having a cross-sectional size smaller than the cross-sectional size of the particular one of the elongated tubular bodies.

27. The carbon dioxide capture apparatus of claim 16 wherein the one or more surfaces of the substrate of all of the carbon dioxide capture devices coated with carbon-dioxide capture coating composition is at least 40 times greater than the planar area occupied by the device retaining structure.

28. The carbon dioxide capture apparatus of claim 27 wherein the one or more surfaces of each of the carbon dioxide capture devices extends vertically within the interior space of the device retaining structure.

29. The carbon dioxide capture apparatus of claim 28 wherein the substrate is made from a material that is light transmissive.

30. The carbon dioxide capture apparatus of claim 28 wherein the substrate of each of the carbon dioxide capture devices is generally flat.
